(19) United States
(12) Patent Application Publication
(10) Pub. No.: US 2006/0024672 A1
(43) Pub. Date: Feb. 2, 2006

(54) Verification of food origin based on nucleic acid pattern recognition

(76) Inventors: Oystein Lie, Oslo (NO); Audun Slettan, Holum (NO); Morten Hoyum, Ski (NO); Frode Lingaas, Fagernes (NO)

Correspondence Address: MCDERMOTT, WILL & EMERY, 4370 LA JOLLA VILLAGE DRIVE, SUITE 700, SAN DIEGO, CA, 92122, US

(21) Appl. No.: 10/349,331

(22) Filed: Jan. 21, 2003

Related U.S. Application Data
(60) Provisional application No. 60/349,950, filed on Jan. 18, 2002.
(60) Provisional application No. 60/404,200, filed on Aug. 16, 2002.

(51) Int. Cl. C12Q 1/68; C07H 21/04

(52) U.S. Cl. 435/6; 536/24.3

(57) ABSTRACT

This invention is directed to isolated nucleic acid molecules that encompass a single nucleotide polymorphism (SNP) associated with fish. The present invention further is directed to isolated nucleic acid molecules that encompass a microsatellite sequence associated with fish. The invention further is directed to a method of determining the parentage origin of a fish sample (or a sample from any biological species with similar organization of reproduction as fish) by providing a parentage genotype database that contains a collection of candidate parent genotypes that each represent a distinct parentage origin and comparing a sample genotype to the parentage genotype database, such that a match between a sample genotype and one of the candidate parent genotypes identifies the parentage origin of the sample.

### BACKGROUND OF THE INVENTION

This application claims benefit of the filing date of U.S. Provisional Application No. 60/349,950, filed Jan. 18, 2002, and 60/404,200, filed Aug. 16, 2002, and which are incorporated herein by reference.

This invention relates generally to applied genomics methods and, more specifically, to methods for determining the source of a fish sample.

Increased focus has been placed on healthy food, and consumers are increasingly concerned with core issues such as sustainable and environmentally safe harvest and production processes, the use of drugs and feed additives as well as the welfare of the production animals. Governmental authorities, seafood retail traders and consumers presently have no available system to verify whether the production process is in accordance with information provided, whether the product has the origin as claimed or whether, for example, a fillet in the supermarket has the correct brand name.

Seafood operators are becoming increasingly aware of the importance of implementing quality control mechanisms together with traceability systems for the purpose of establishing verifiable substance in order to protect their products and brand names. Similarly, retailers and consumers want to be able to check whether they have received the desired product or brand in accord with the claimed quality.

Presently existing traceability systems are unreliable as they depend on “paper flow” along the value chain to provide information regarding origin; production parameters; processing time, date and environment; and transport. Consequently, there is a need for an authenticity system verifying the origin of products at high speed and low cost.

Several reasons support the need of a genetic online traceability system. First, consumers growing concern with regard to core issues like the health risk of consuming a particular product. Furthermore, consumers are increasingly concerned with whether a product has been subjected to resource and environmentally friendly harvest and production as well as with animal welfare issues. In addition to these consumer demands, recent regulations passed in the United States and the European Union focus on environmentally friendly harvest and production. Significantly, each of the foregoing issues is related to product origin.

Thus, there exists a need for genetic markers that can be used to unambiguously and reliably identify the origin of a fish sample and for methods to efficiently determine the origin of a fish sample using such markers. The present invention satisfies this need and provides related advantages as well.

### SUMMARY OF THE INVENTION

This invention is directed to isolated nucleic acid molecules that encompass a single nucleotide polymorphism (SNP) associated with fish. The present invention further is directed to isolated nucleic acid molecules that encompass a microsatellite sequence and corresponding primers associated with fish. The invention also provides nucleotide sequences corresponding to Polymerase Chain Reaction (PCR) primers, Oligonucleotide Ligation Assay (OLA) primers. The polymorphism nucleotide sequences and corresponding primers provided by the present invention are described below, designated SEQ ID NOS:1-1377, and set forth in FIGS. 1 through 9 and 11.

The invention further is directed to a method of determining the parentage origin of a fish sample by providing a parentage genotype database that contains a collection of candidate parent genotypes that each represent a distinct parentage origin and comparing a sample genotype to the parentage genotype database, such that a match between a sample genotype and one of the candidate parent genotypes identifies the parentage origin of the sample. The invention also provides a method of determining the origin of a fish sample by providing an origin genotype database encompassing a collection of candidate genotype profiles, wherein each of the candidate genotype profiles represents a distinct population of origin; and comparing a sample genotype to the candidate genotype profiles, wherein a match between the sample genotype and one of the candidate genotype profiles identifies the population of origin of the sample.

### BRIEF DESCRIPTION OF THE DRAWINGS

FIG. 1 shows the nucleotide sequences of Salmo salar Single Nucleotide Polymorphisms (SNPs) and corresponding OLA primers (SEQ ID NOS: 1-112).

FIG. 2 shows the nucleotide sequences of Polymerase Chain Reaction (PCR) primers corresponding to Salmo salar Single Nucleotide Polymorphisms (SNPs) (SEQ ID NOS: 113-154).

FIG. 3 shows the nucleotide sequences of Salmo salar microsatellites (SEQ ID NOS: 155-164).

FIG. 4 shows the nucleotide sequences of Orechromis niloticus Single Nucleotide Polymorphisms (SNPs) and corresponding OLA and SNP primers (SEQ ID NOS: 165-308).

FIG. 5 shows the nucleotide sequences of Orechromis niloticus microsatellites (SEQ ID NOS: 309-367).

FIG. 6 shows the nucleotide sequences of Orechromis niloticus polymorphic sites (SEQ ID NOS: 368-373).

FIG. 7 shows the nucleotide sequences of

Atlantic halibut Single Nucleotide Polymorphism (SNPS) (SEQ ID NOS: 374-409).

FIG. 8 shows the nucleotide sequences of cod polymorphic sites (SEQ ID NOS: 410-414).

FIG. 9 shows the nucleotide sequences of seabass polymorphic sites (SEQ ID NOS: 415-472).

FIG. 10 shows a schematic illustration of the invention method for determining the parentage origin of a fish sample.

FIG. 11 shows nucleotide sequences of Oreochromis niloticus microsatellites and corresponding primers (SEQ ID NOS: 473-1377).

### DETAILED DESCRIPTION OF THE INVENTION

This invention is directed to isolated nucleic acid molecules that encompass a single nucleotide polymorphism (SNP) associated with several distinct species of fish. The present invention further is directed to isolated nucleic acid molecules that encompass a microsatellite sequence associated with several distinct species of fish. Also provided are methods for determining the parentage origin or population of origin of a sample based on matching of genetic markers.

As used herein, the term “fish,” refers to organisms falling into one of two groups, “cartilagenous fish” or class Chondrichthyes and “bony fish” or class Osteichthyes (formerly class name, but still widely used). Most of the modern Osteichthyes belong the order Teleostei.

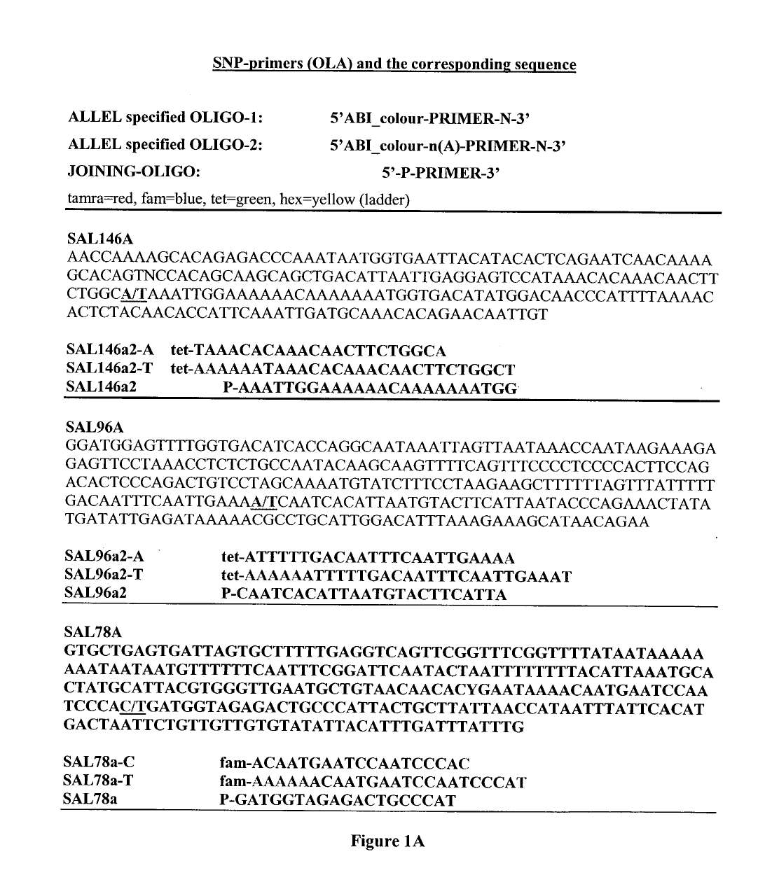

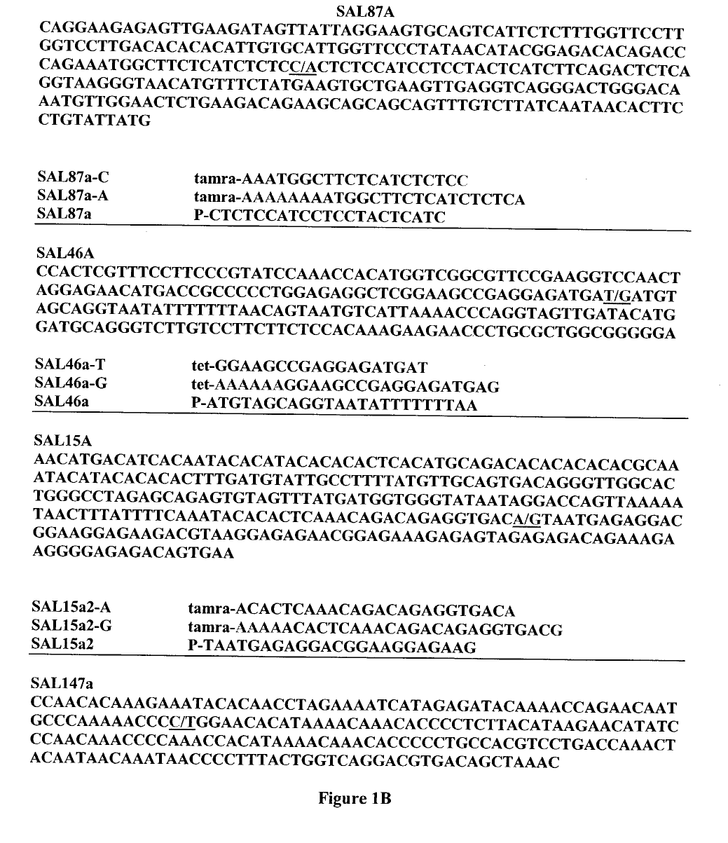

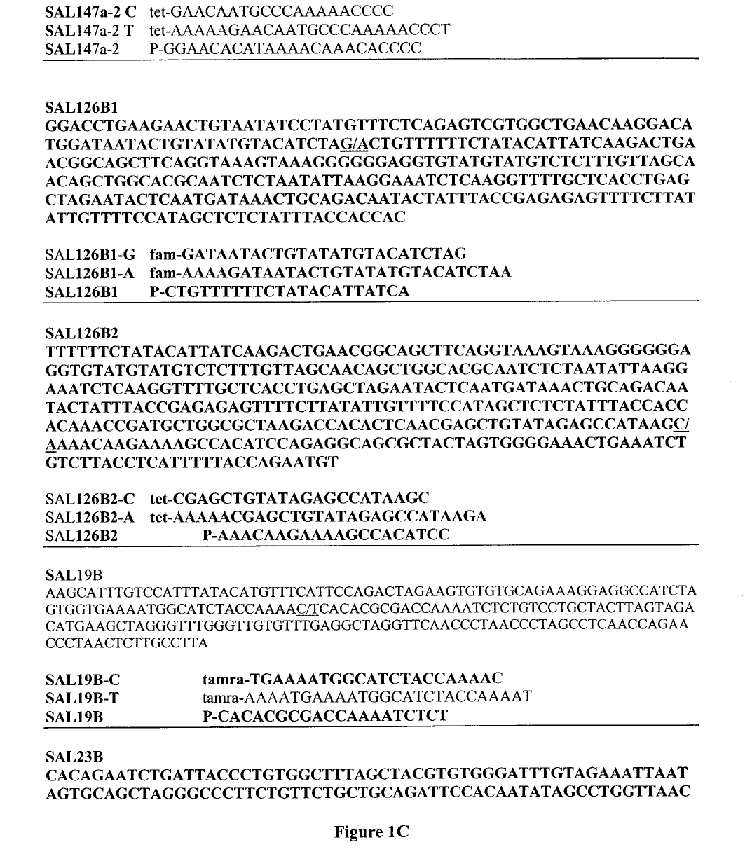

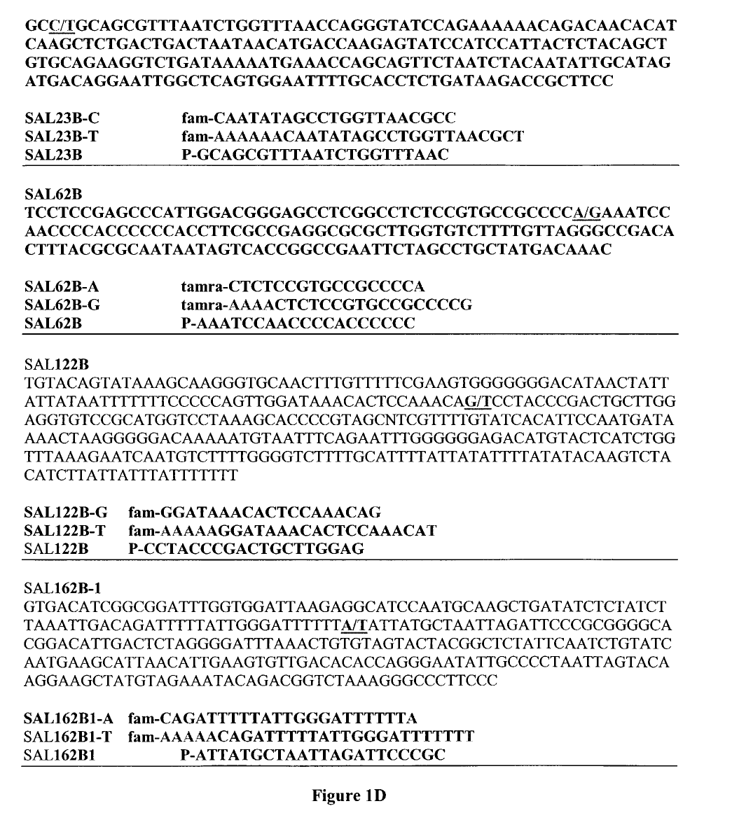

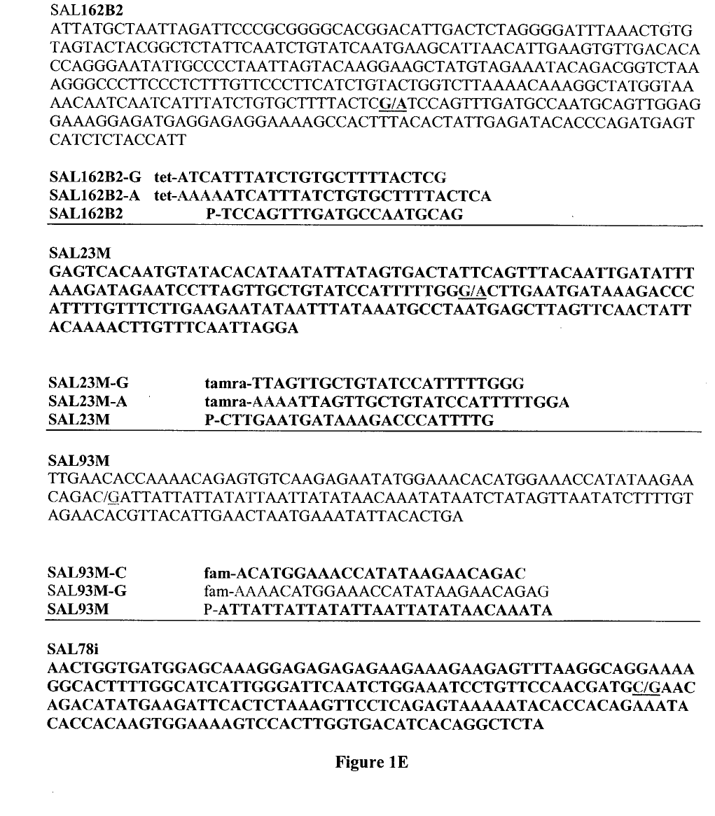

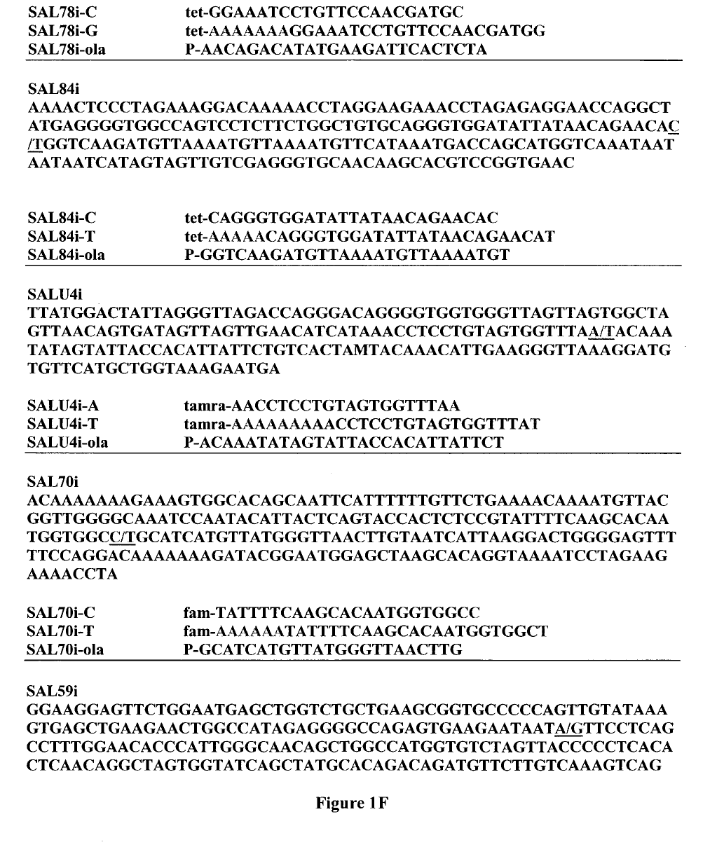

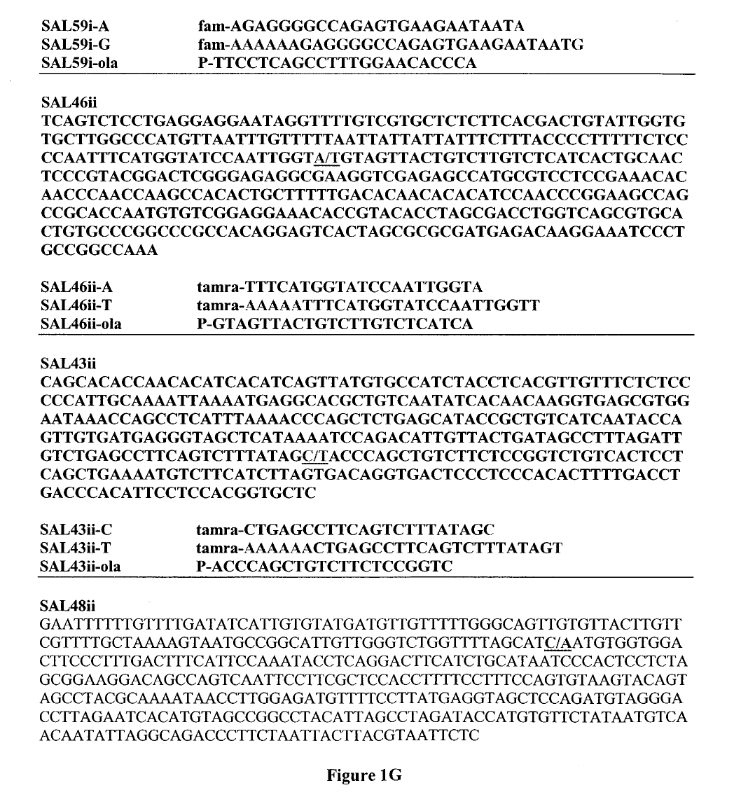

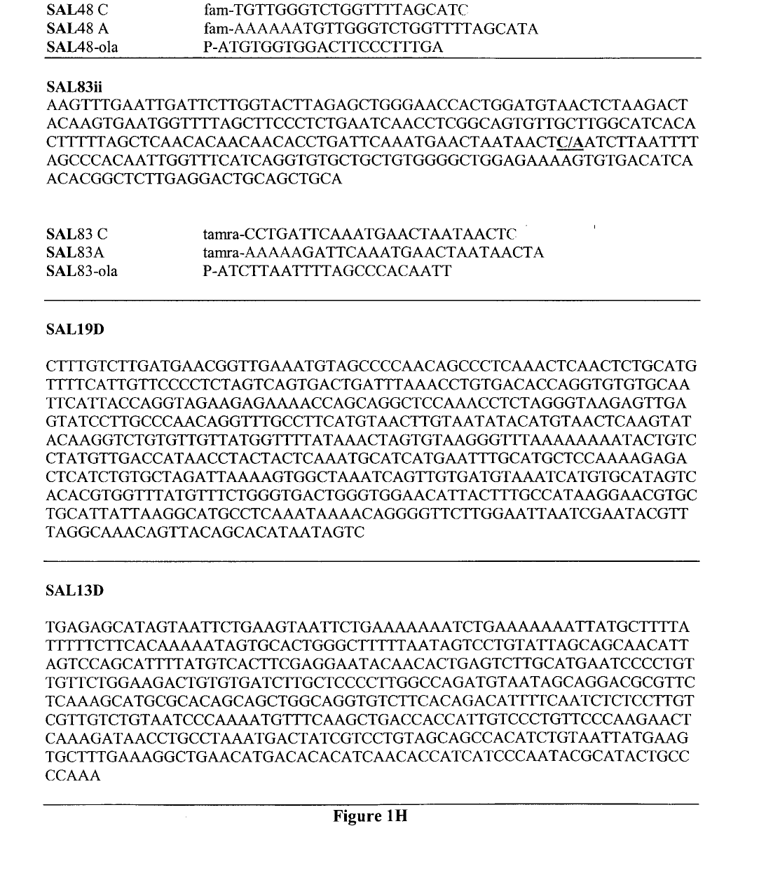

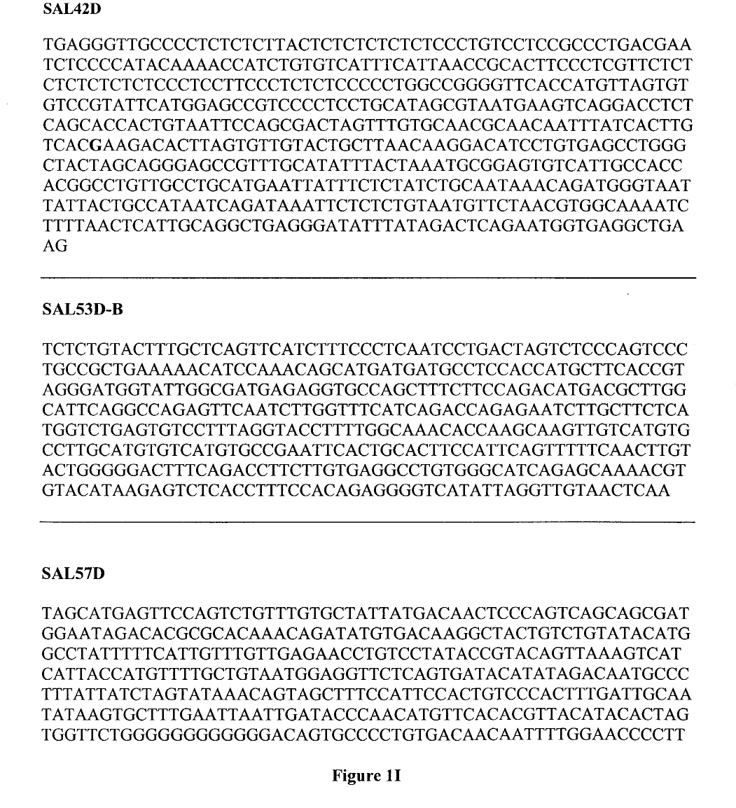

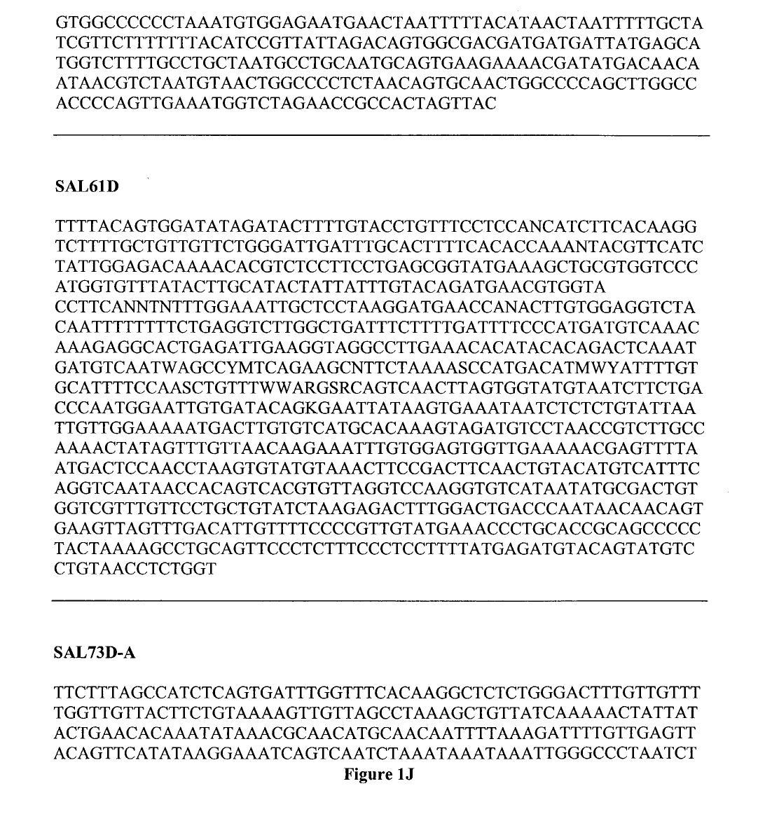

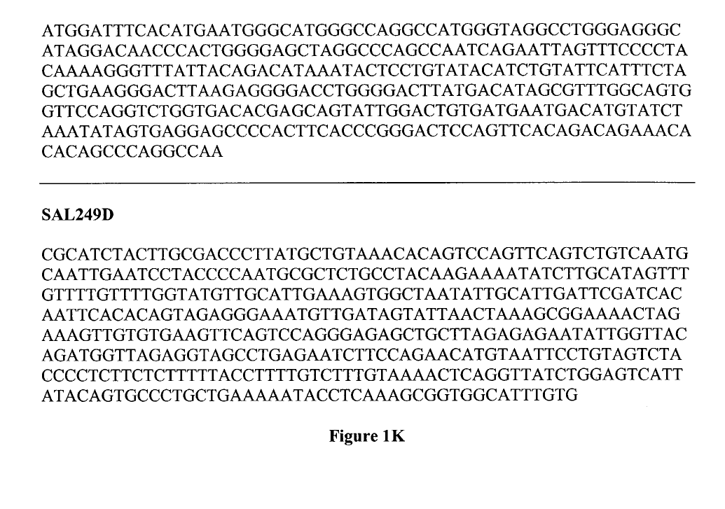

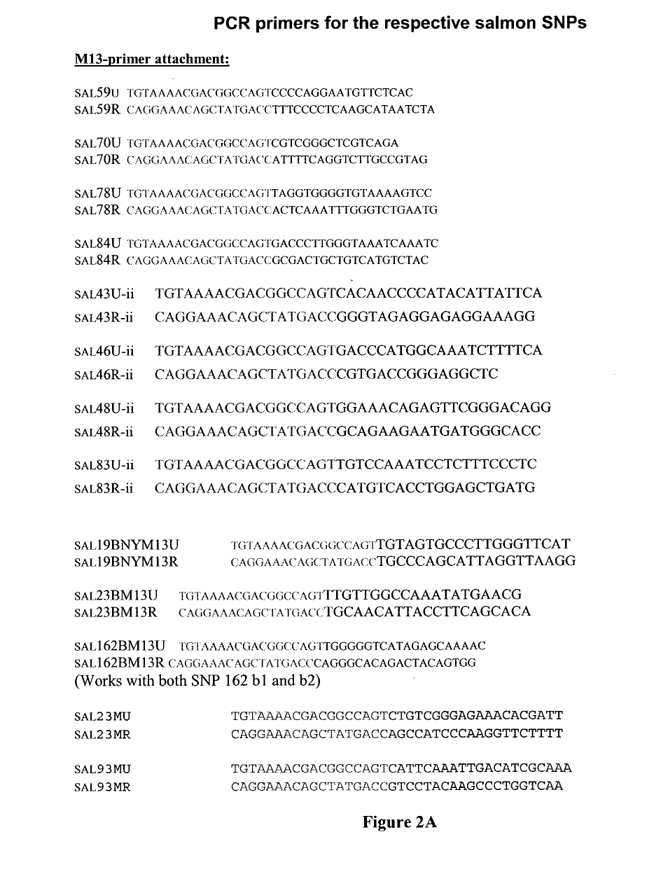

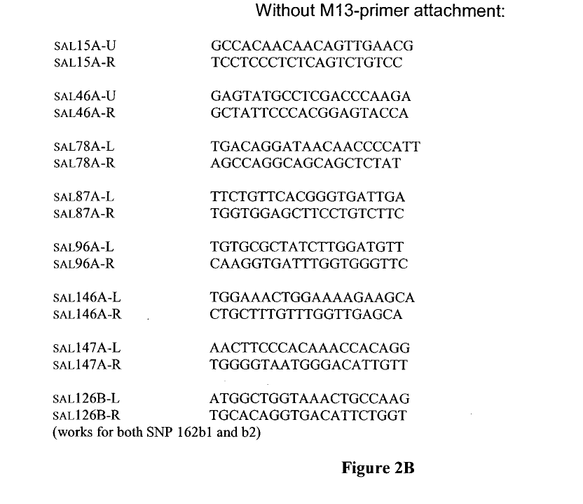

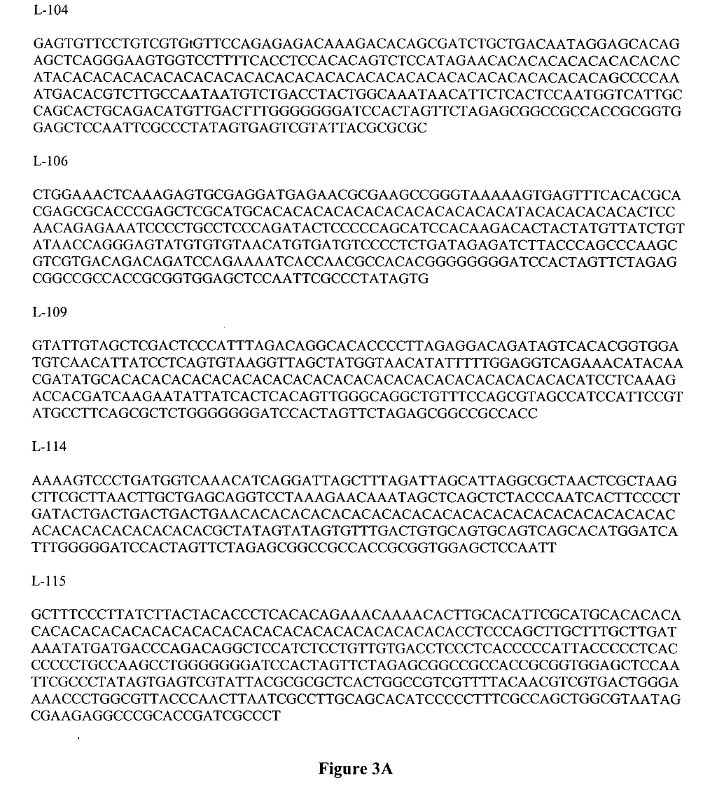

In one embodiment, the invention provides an isolated nucleic acid molecule encompassing a single nucleotide polymorphism (SNP), where the isolated nucleic acid molecule is selected from the group set forth in FIG. 1, which correspond to the order Salmoniformes, family Salmonidae, genus Salmo and species Salmo salar. Also provided are nucleic acid molecules that hybridize to the nucleic acid molecule selected from the group set forth in FIG. 1 or its complement under highly stringent hybridization conditions. FIG. 1 shows isolated nucleic acid molecules encompassing a single nucleotide polymorphism (SNP) and corresponding OLA primers consecutively designated as SEQ ID NOS: 1-112, which correspond to the order Salmoniformes, family Salmonidae, genus Salmo and species Salmo salar. FIG. 2 shows isolated nucleic acid molecules that represent PCR primers corresponding to Salmo salar single nucleotide polymorphism (SNP) (SEQ ID NOS: 113-154).

As used herein, the term “salmon,” refers to organisms belonging to the order Salmoniformes, family Salmonidae, genus Salmo and species Salmo salar. All salmonids live in freshwater or migrate into freshwater to spawn in the streams of their origins. Salmo salar is the main species in northern Europe and North America and also the main species of farmed salmon. Worldwide production of farmed salmon has exceeded 800 000 tons per year.

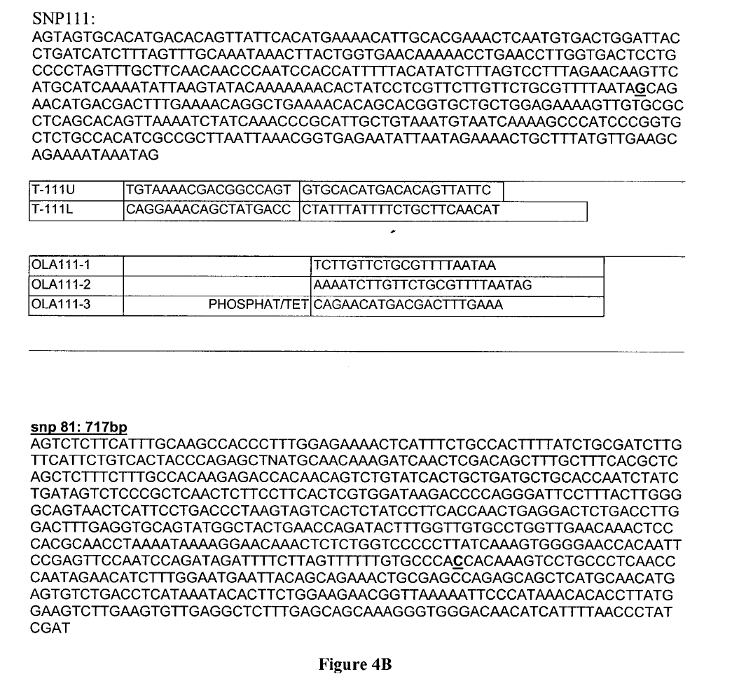

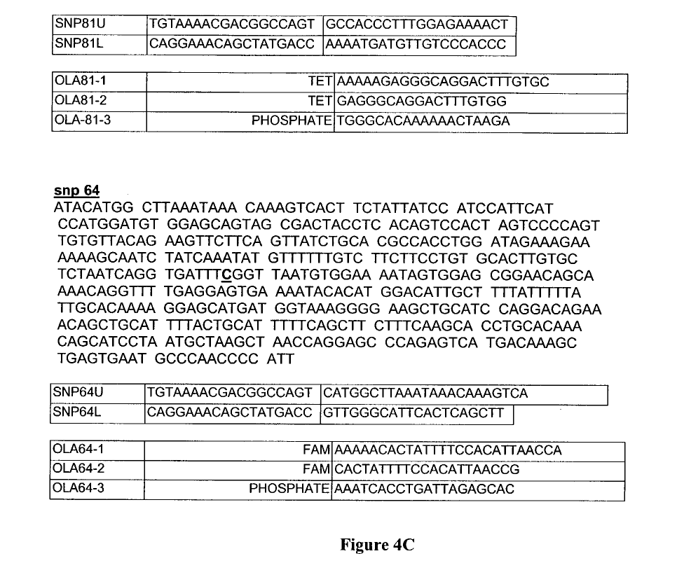

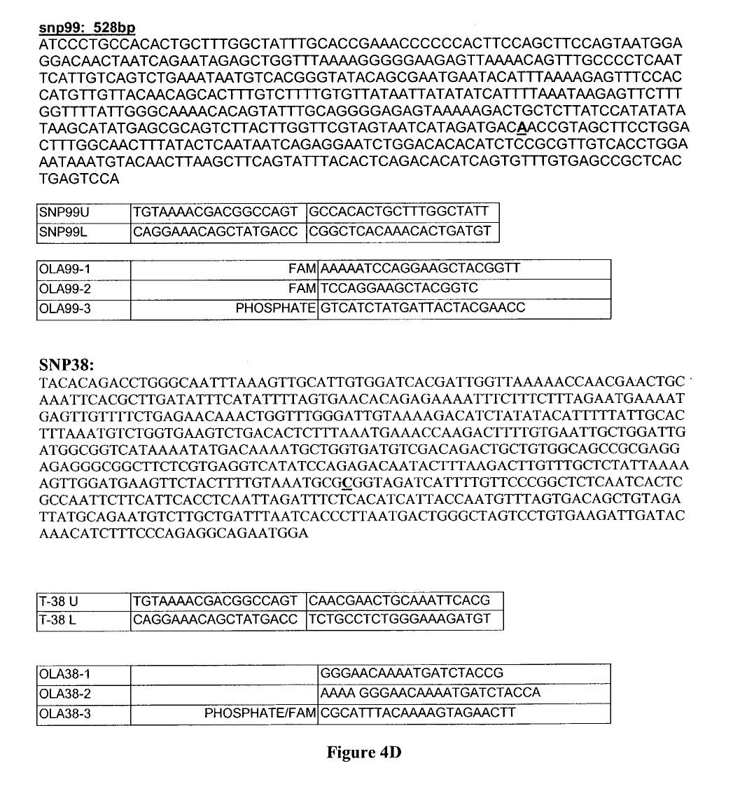

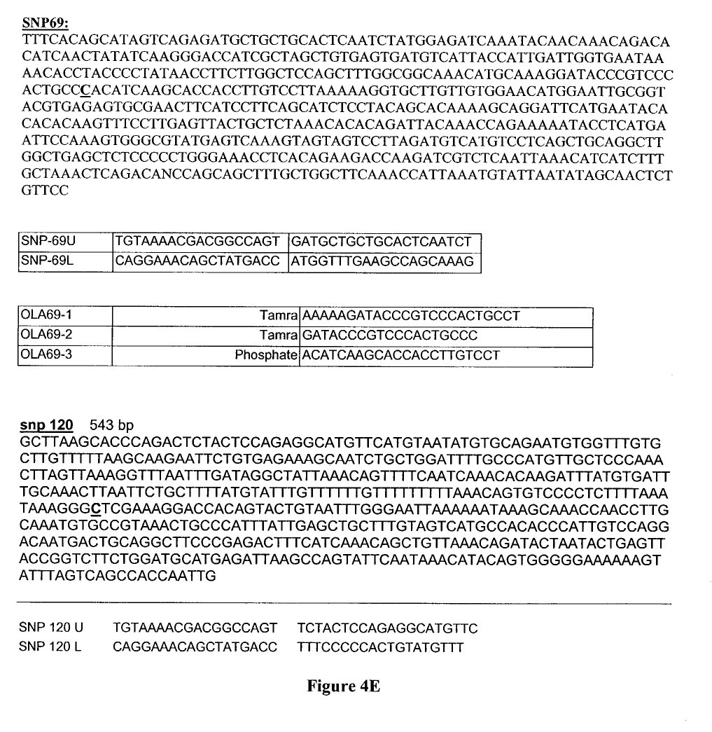

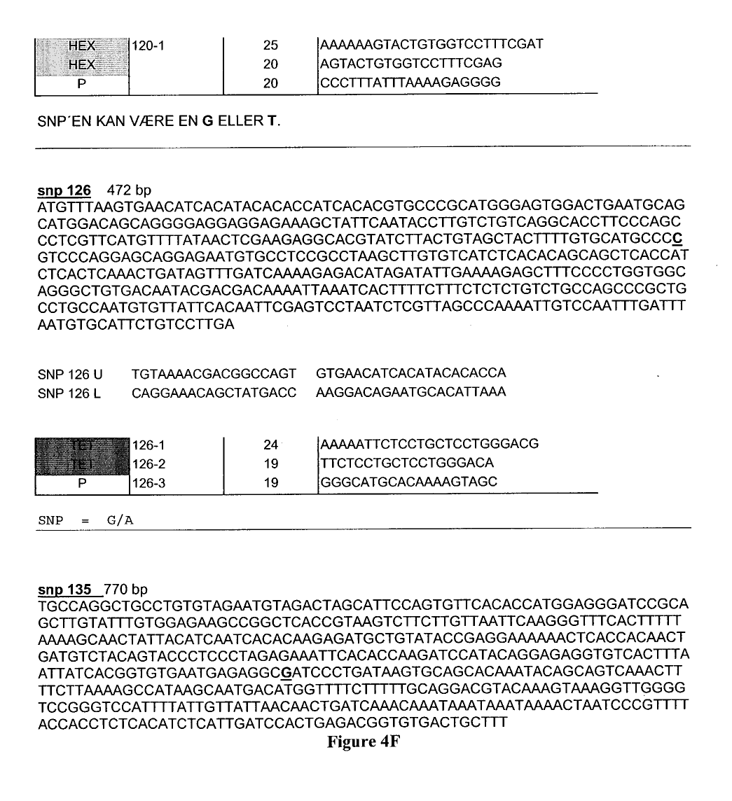

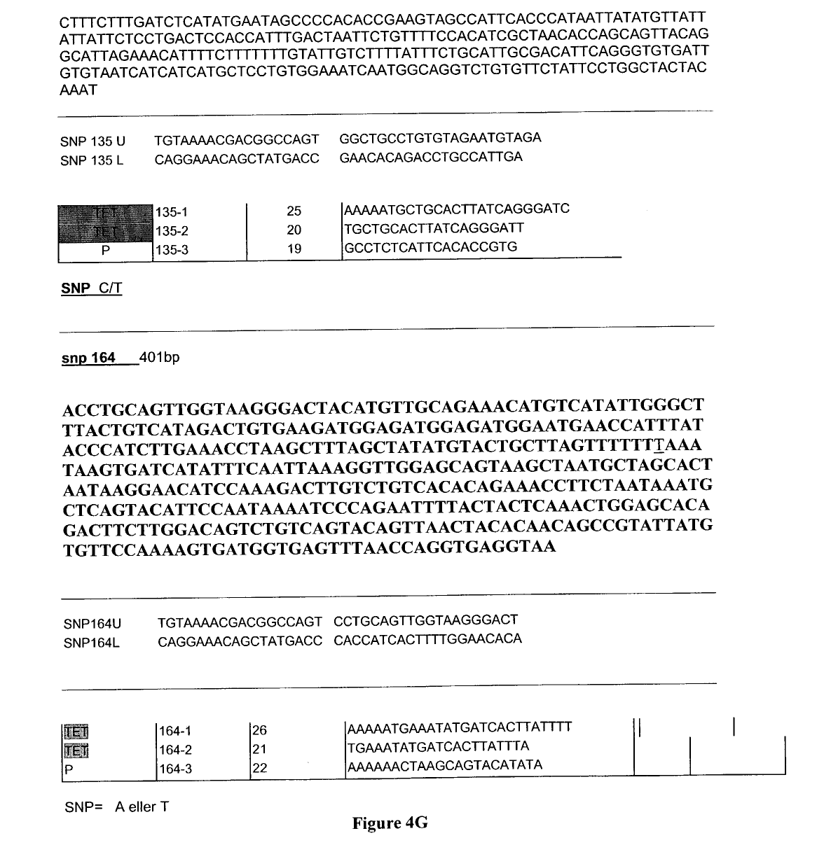

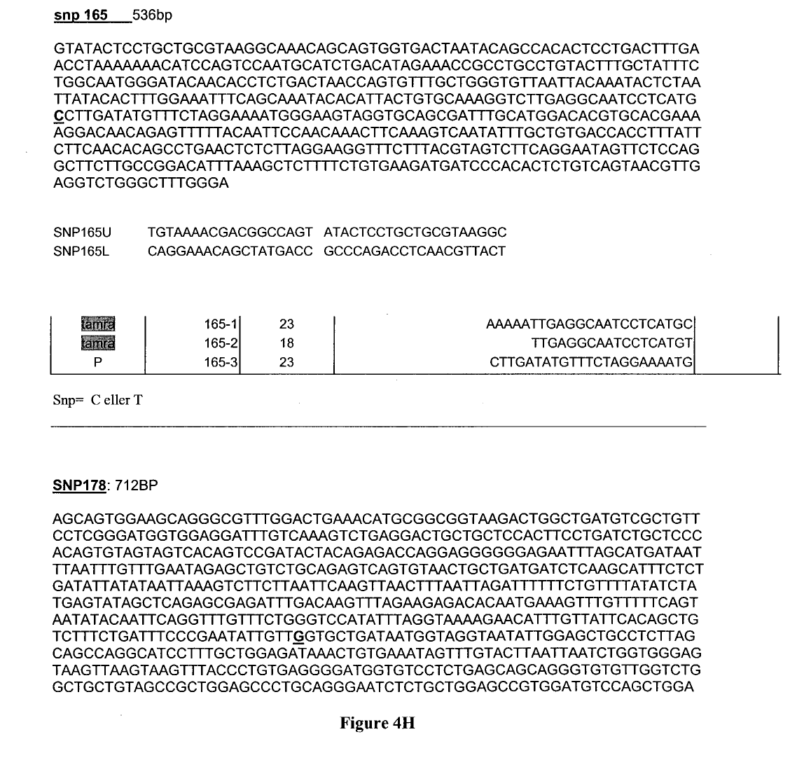

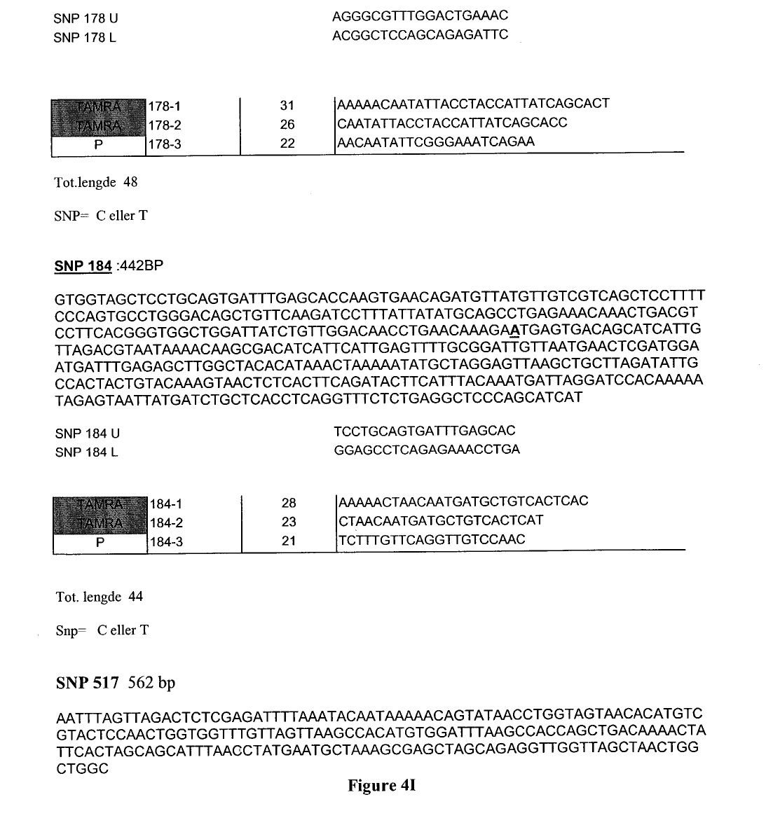

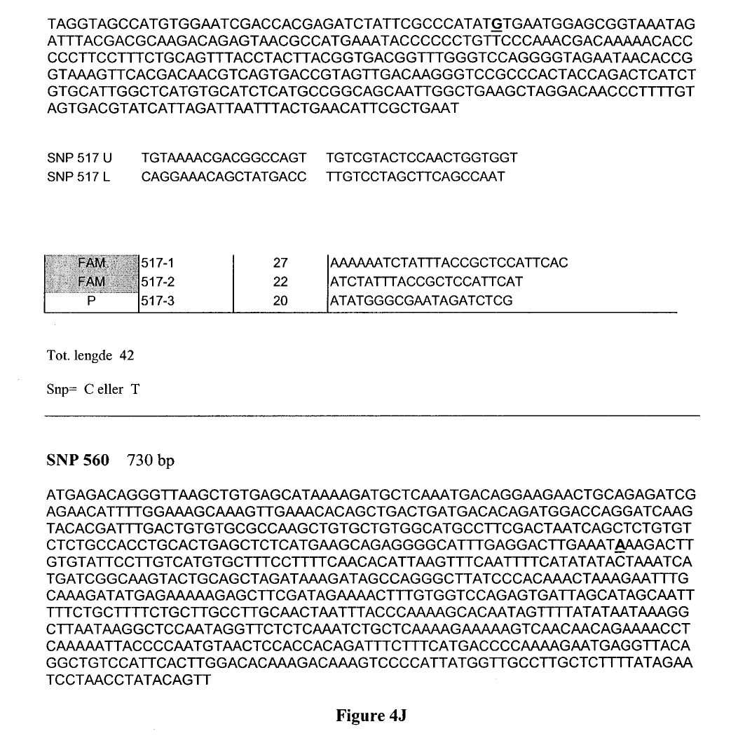

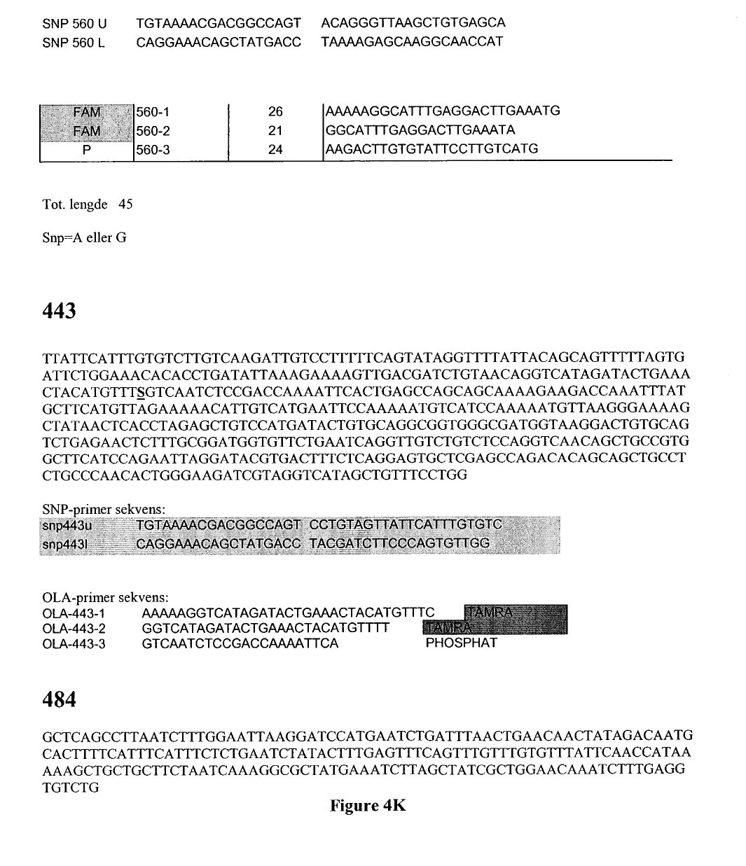

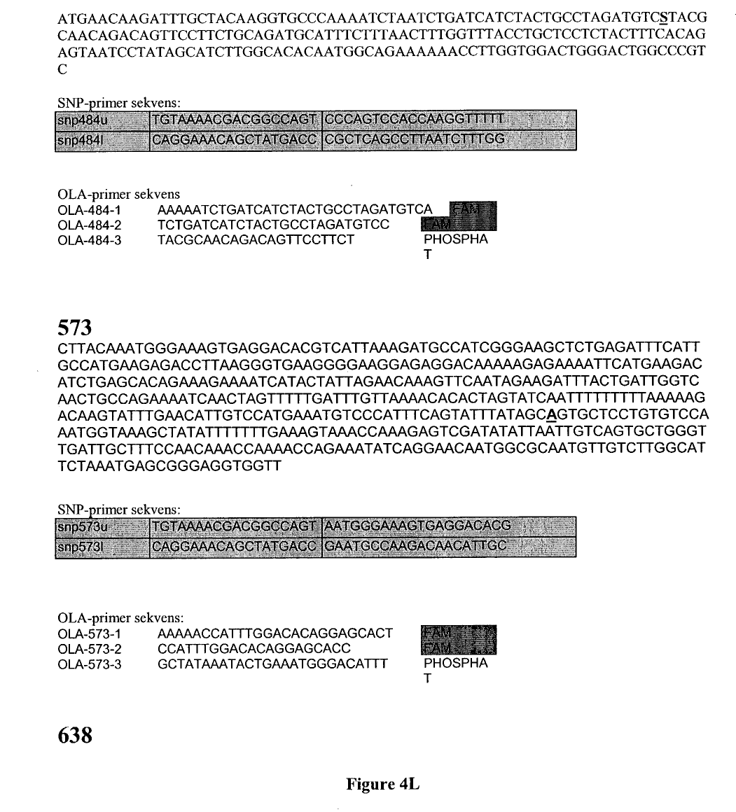

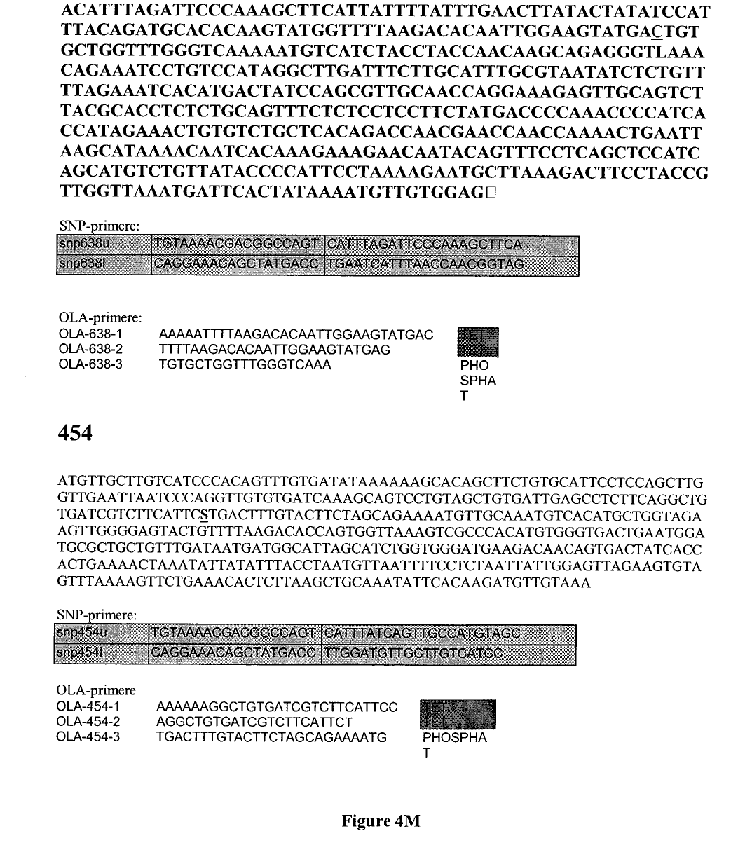

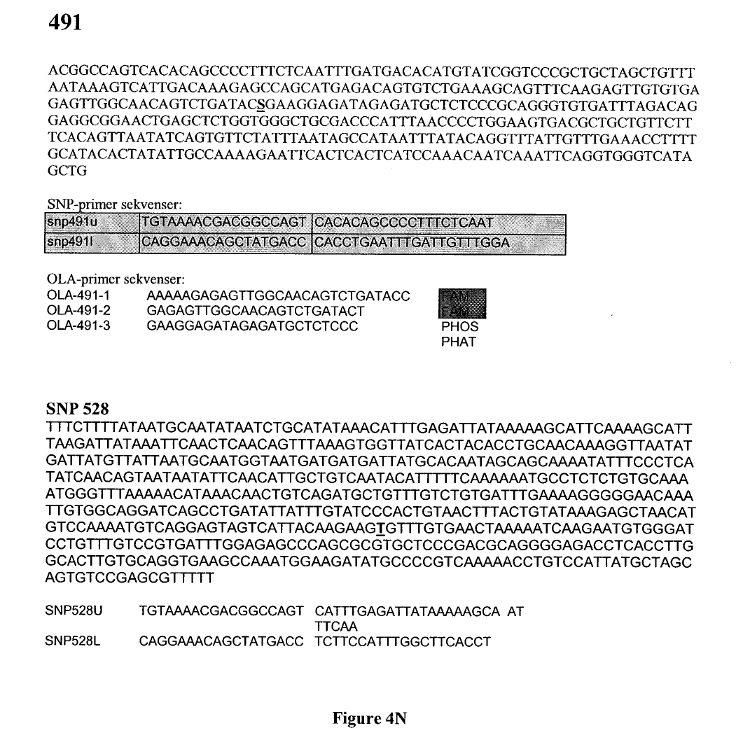

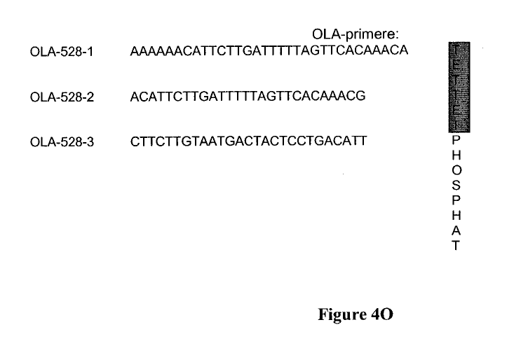

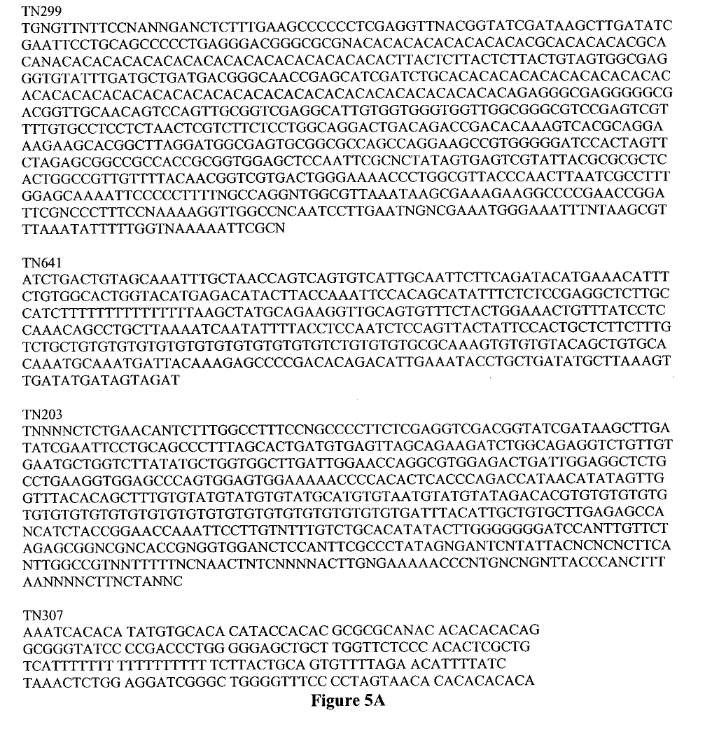

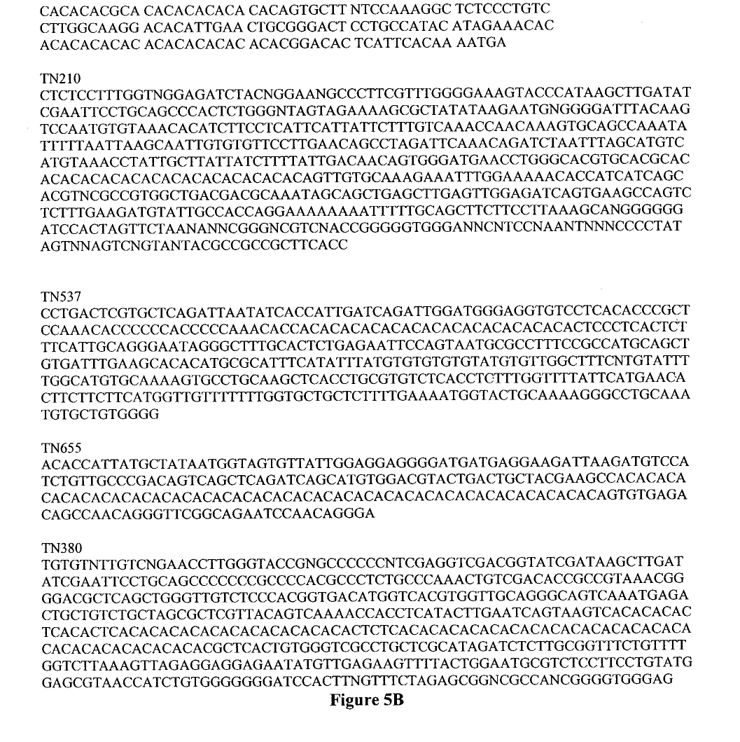

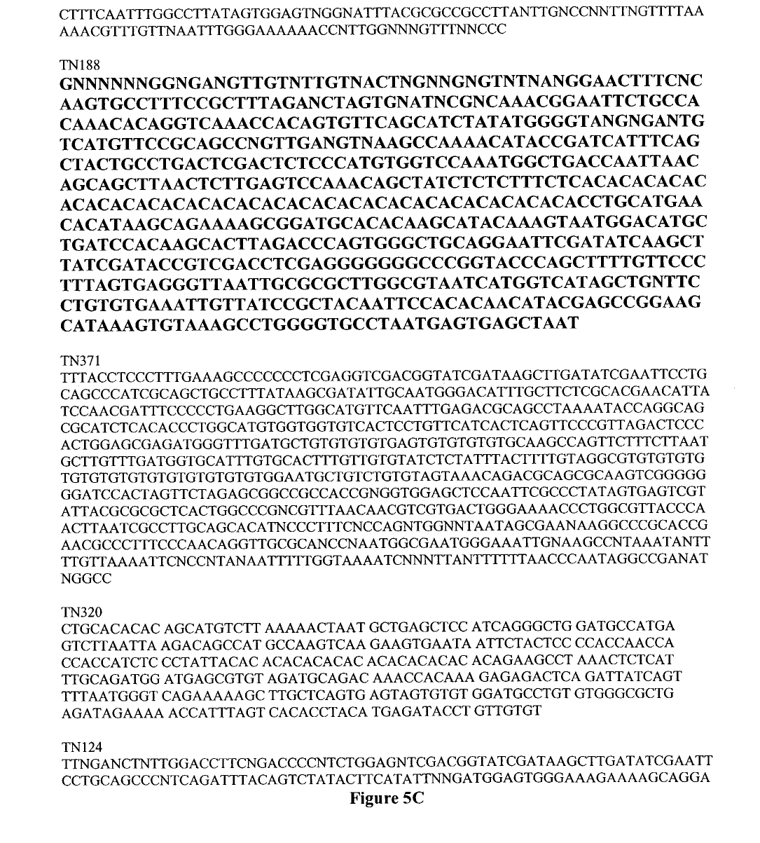

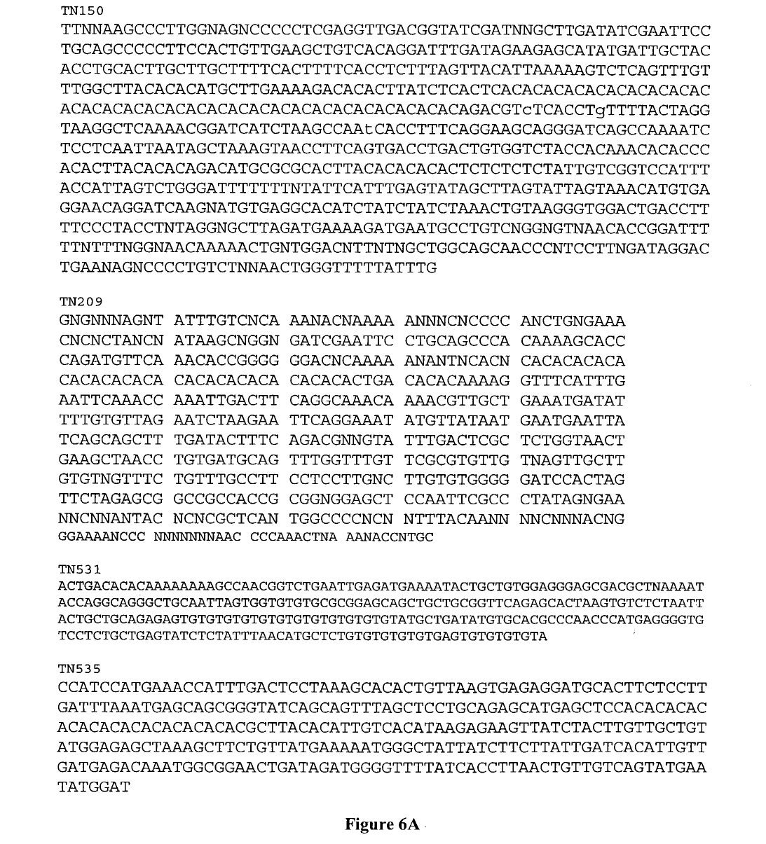

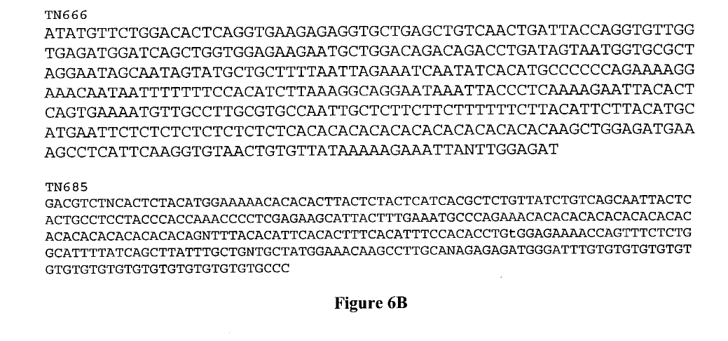

In a further embodiment, the invention provides an isolated nucleic acid molecule encompassing a single nucleotide polymorphism (SNP), where the isolated nucleic acid molecule is selected from the group set forth in FIG. 4, which correspond to the order Perciformes, family Cichlidae, genus Oreochromis and species Oreochromis niloticus. Also provided are nucleic acid molecules that hybridize to the nucleic acid molecule selected from the group set forth in FIG. 4 or its complement under highly stringent hybridization conditions. FIG. 4 shows isolated nucleic acid molecules of the invention encompassing a single nucleotide polymorphism (SNP) as well as corresponding OLA and SNP primer sequences consecutively designated as SEQ ID NOS: 165-308, which correspond to the order Perciformes, family Cichlidae, genus Oreochromis and species Oreochromis niloticus. FIG. 6 shows further isolated nucleic acid molecules of the invention encompassing a polymorhic nucleotide sequence designated as SEQ ID NOS: 368-373, which also correspond to Oreochromis niloticus.

As used herein, the term “tilapia,” refers to organisms belonging to the order Perciformes, family Cichlidae, genus Oreochromis. The species Oreochromis niloticus is the most common tilapia species in modern aquaculture and the majority of isolated nucleotide sequences set forth herein correspond to this species. Most tilapia species belonging to the genus Oreochromis are closely genetically related. Individuals from different tilapia species freely mate with each other, thus making species hybrids that are fertile and often with good production qualities. Furthermore, genetic markers isolated from one tilapia species be used with distinct tilapia species or tilapia hybrids. Therefore, the term “tilapia” refers to organisms belonging to the genus Oreochromis in general.

Tilapia are a group of perch-like fishes of the Cichlidae family that are native to the freshwaters of tropical Africa and represent one of the most important aquatic species in culture today. World-wide production of tilapia exceeds 1 billion pounds per year and production of tilapia in the United States is increasing rapidly.

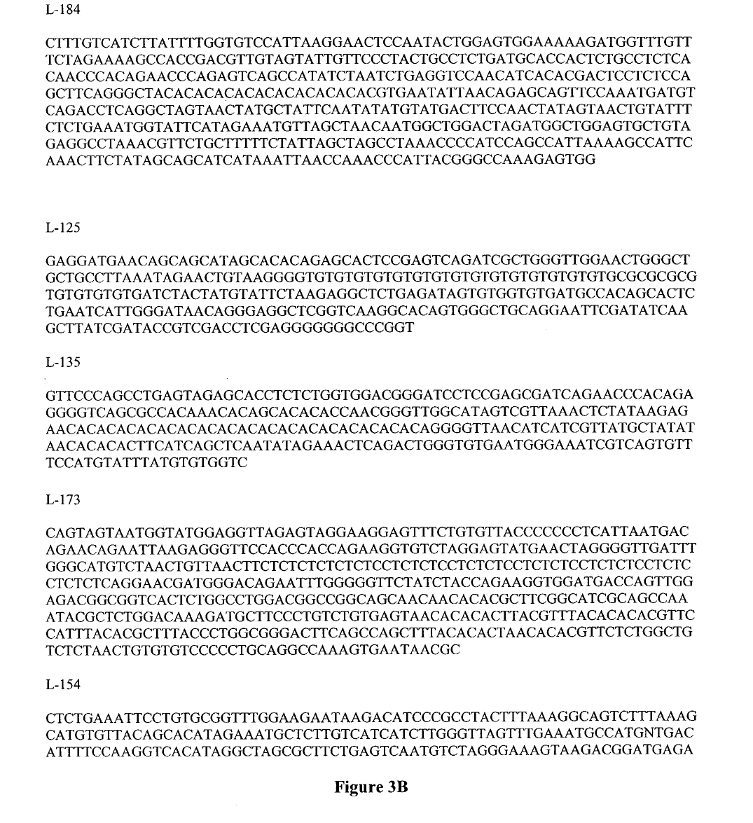

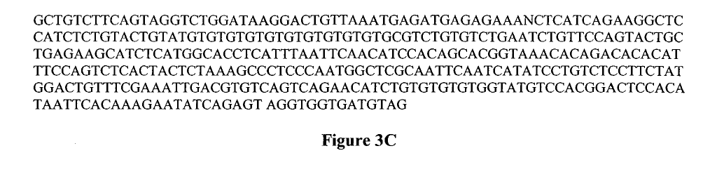

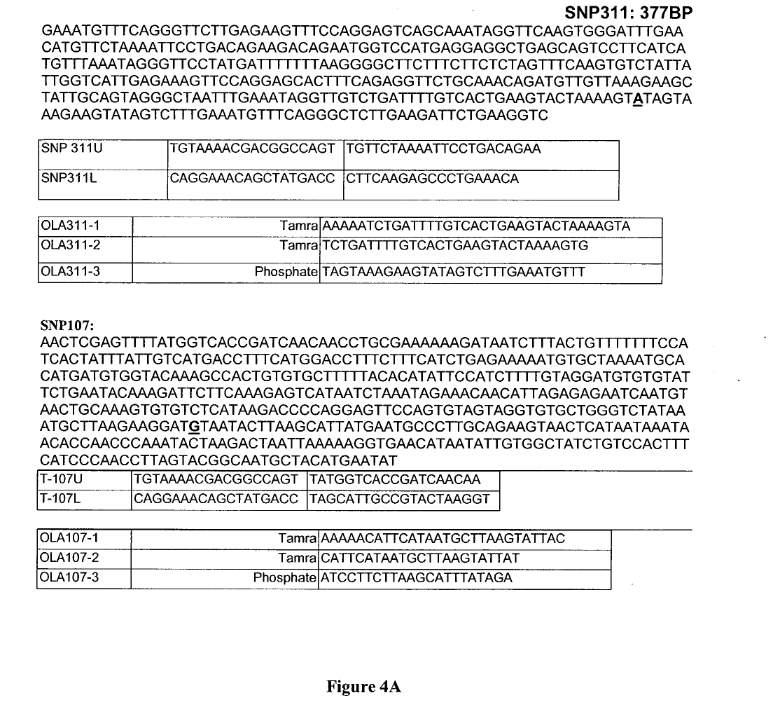

The invention provides isolated nucleic acid molecules that encompass a microsatellite sequence associated with several distinct species of fish. In such an embodiment, the invention provides an isolated nucleic acid molecule encompassing a microsatellite sequence, where the isolated nucleic acid molecule is selected from the group set forth in FIG. 3 and designated SEQ ID NOS: 155-164, which correspond to the salmon. Also provided are nucleic acid molecules that hybridize to the nucleic acid molecule selected from the group designated SEQ ID NOS: 155-164 or its complement under highly stringent hybridization conditions.

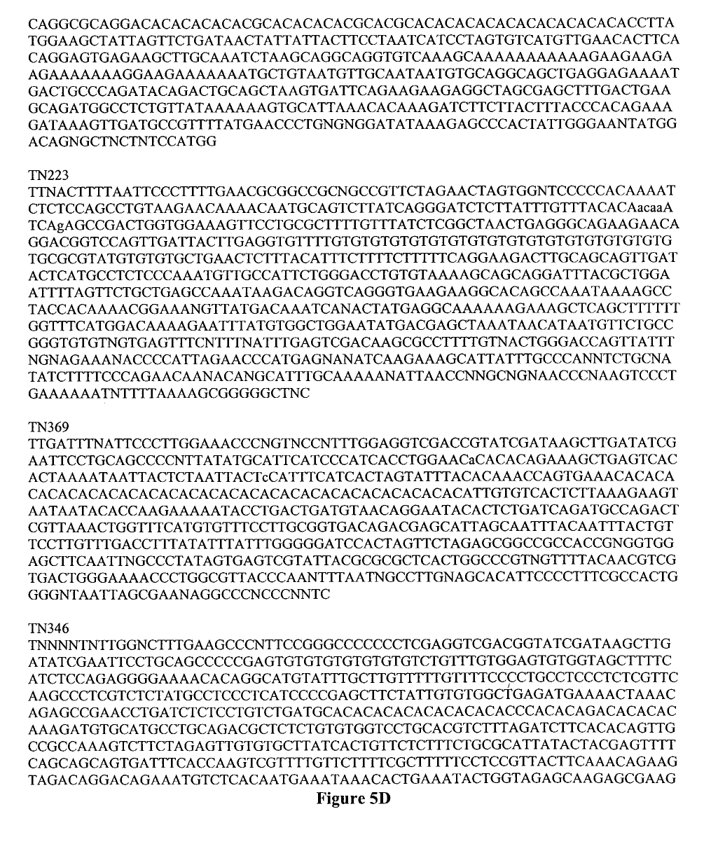

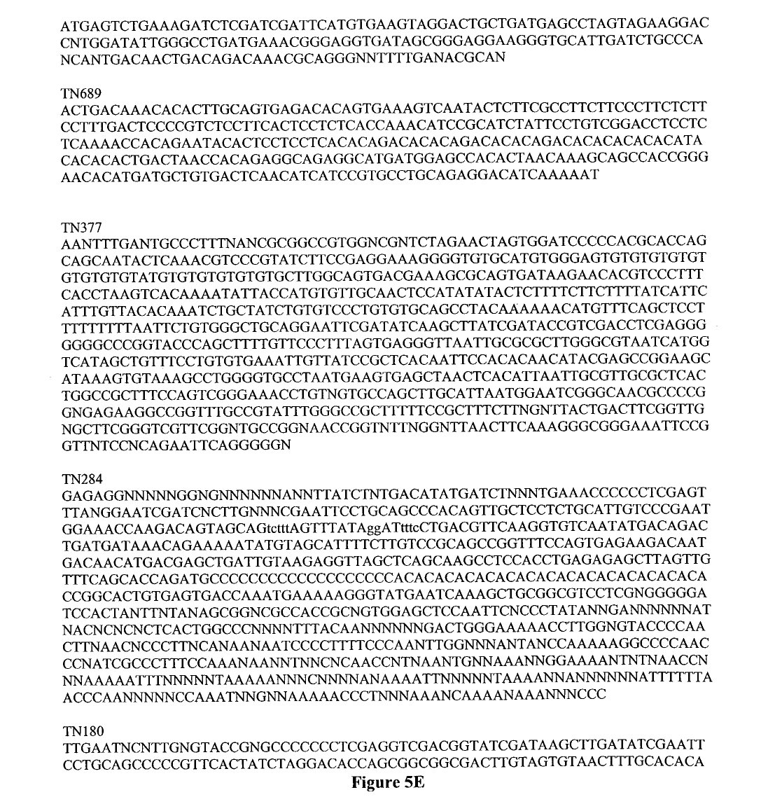

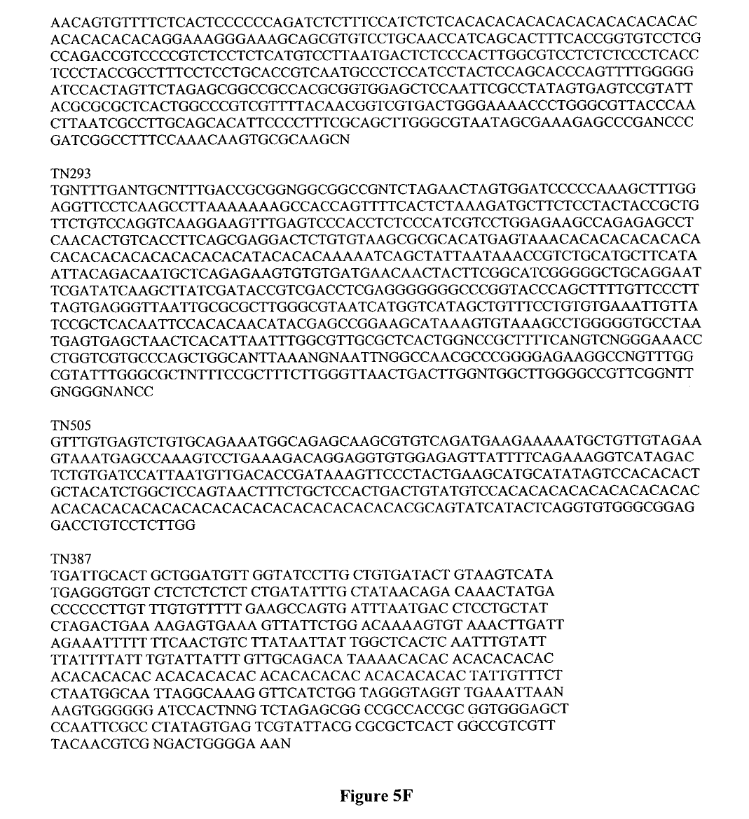

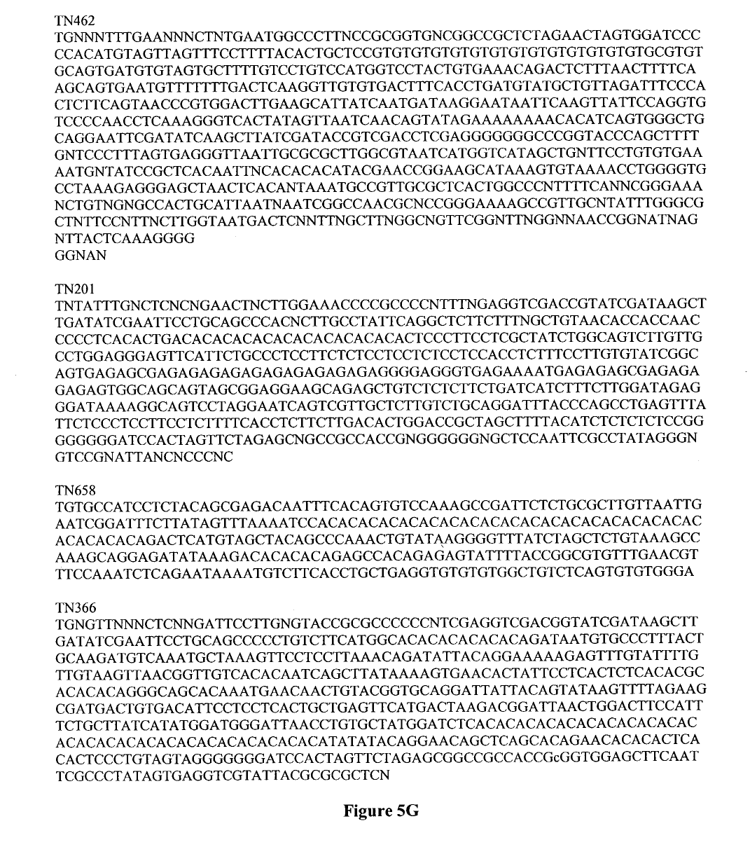

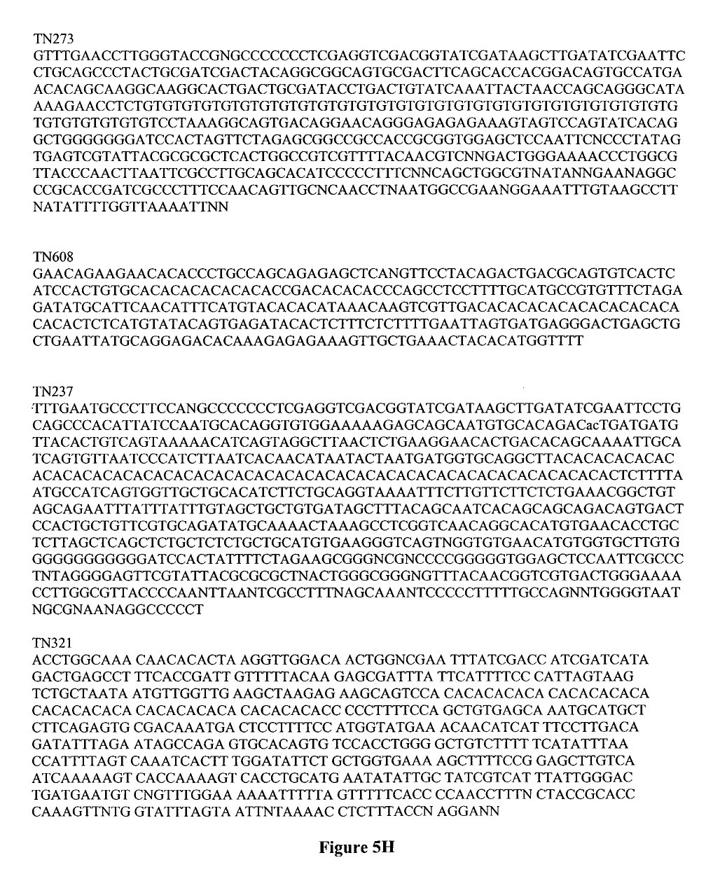

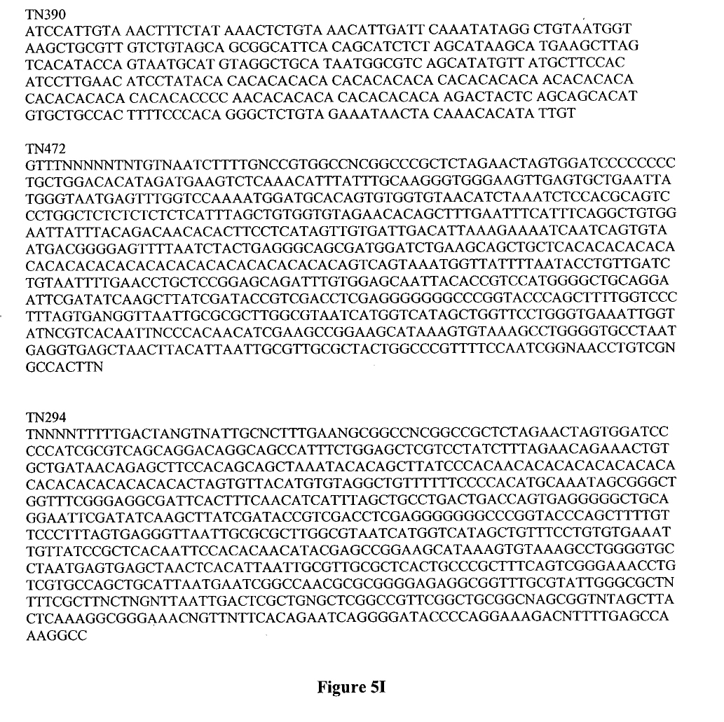

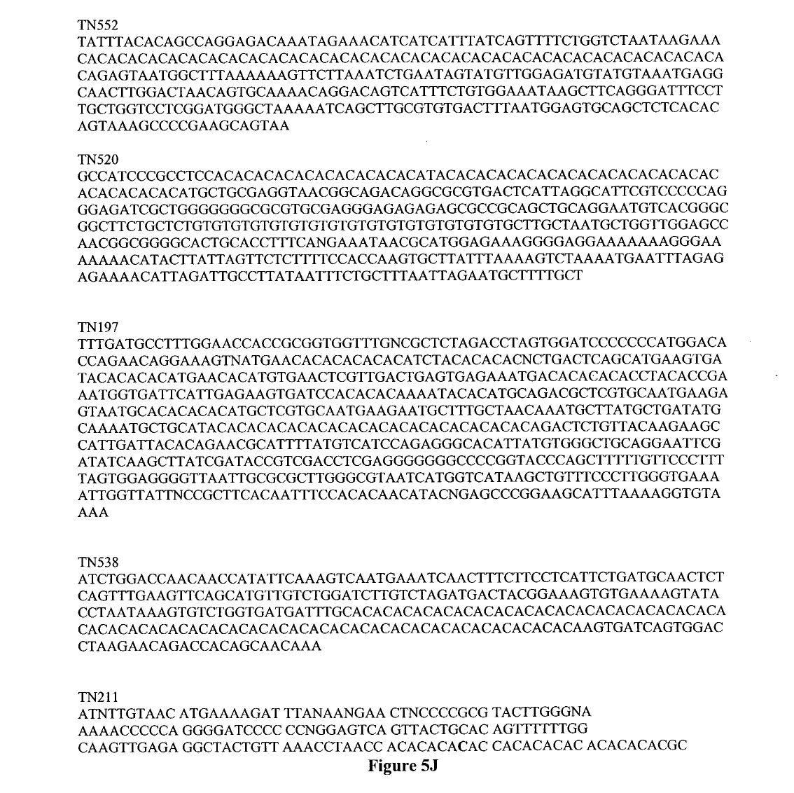

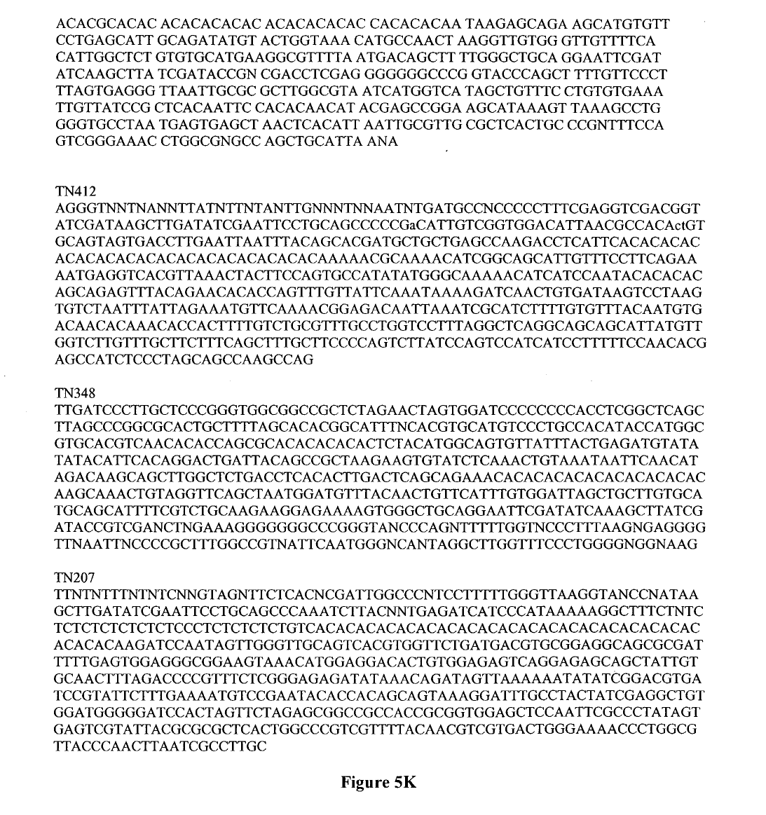

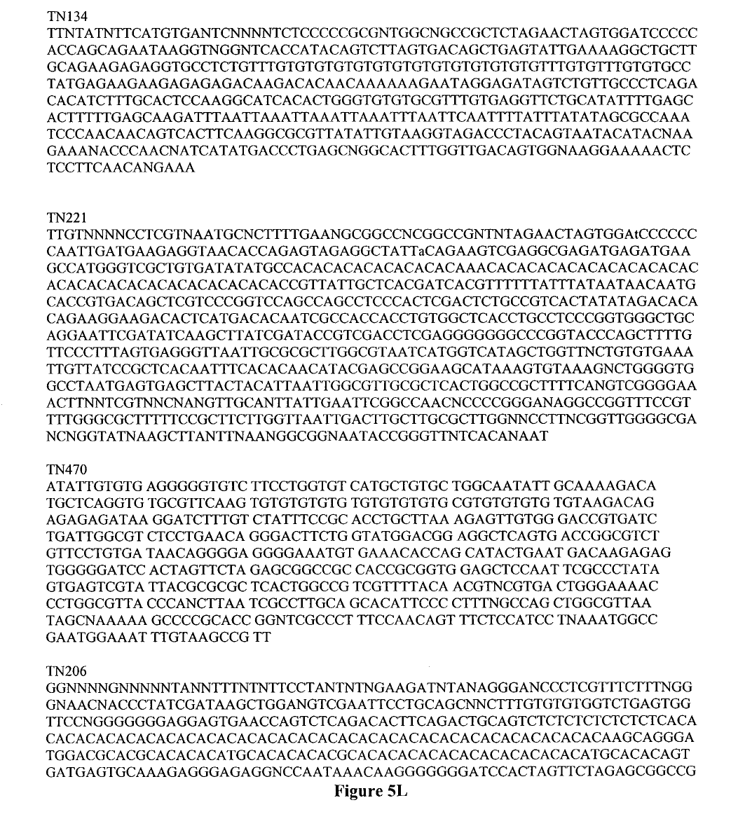

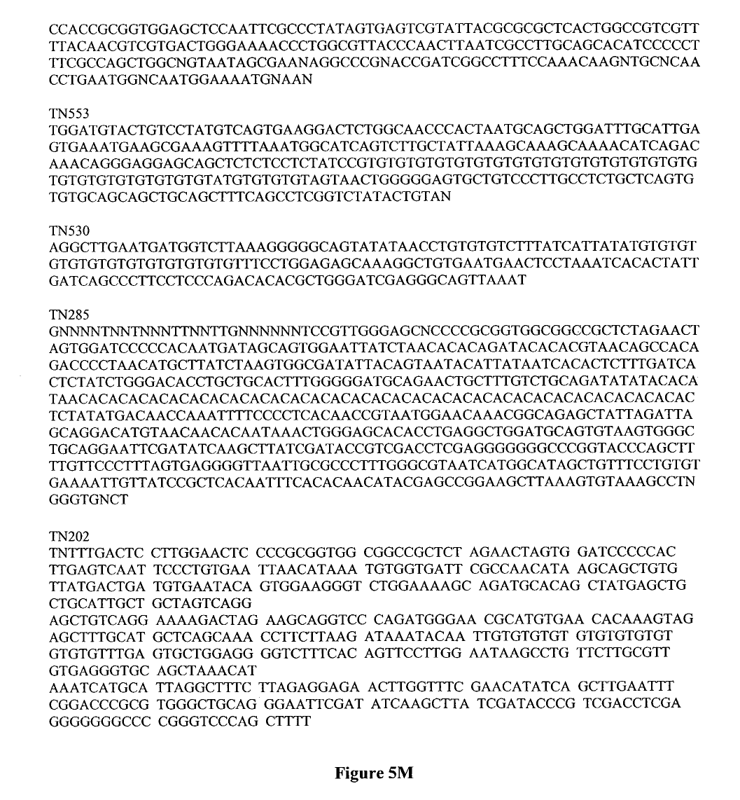

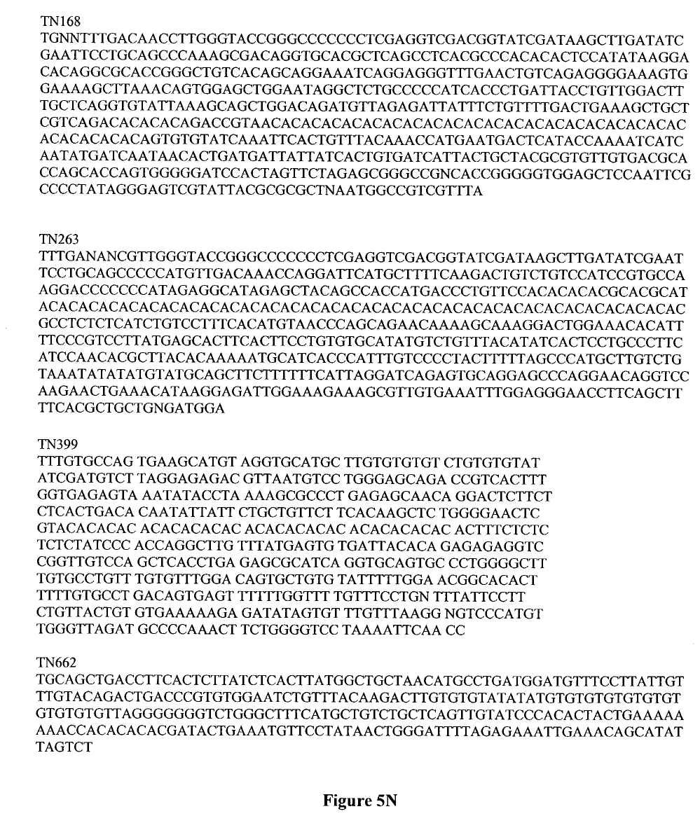

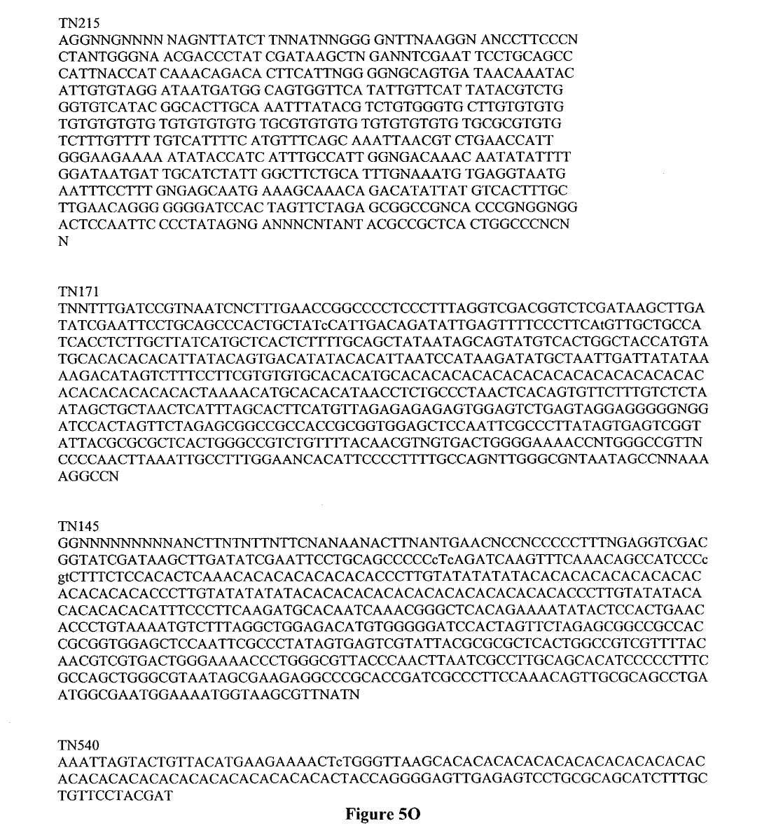

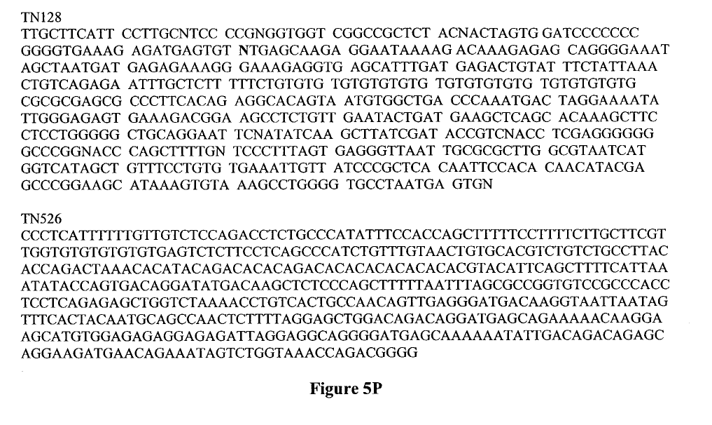

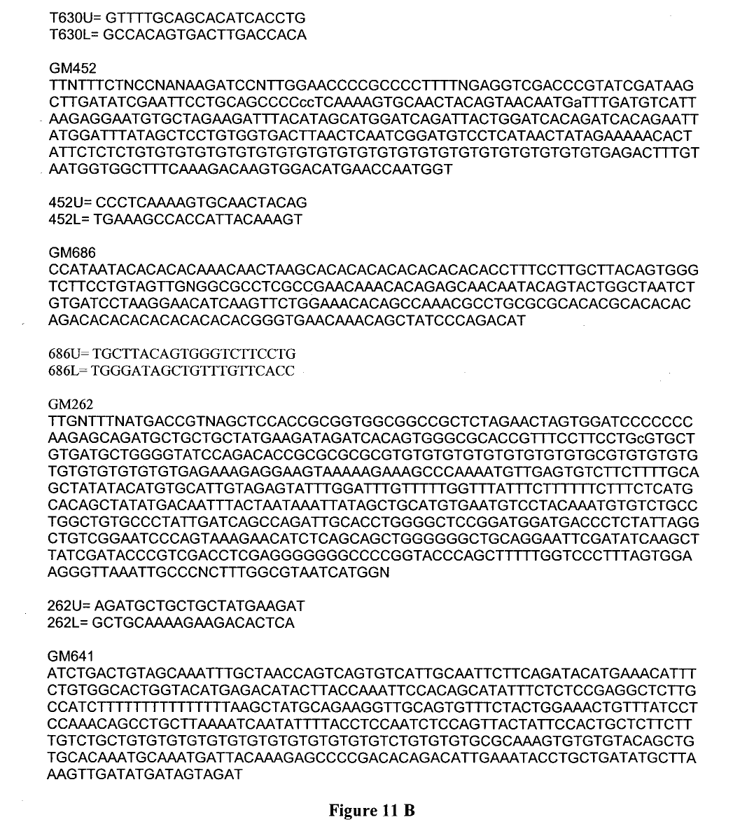

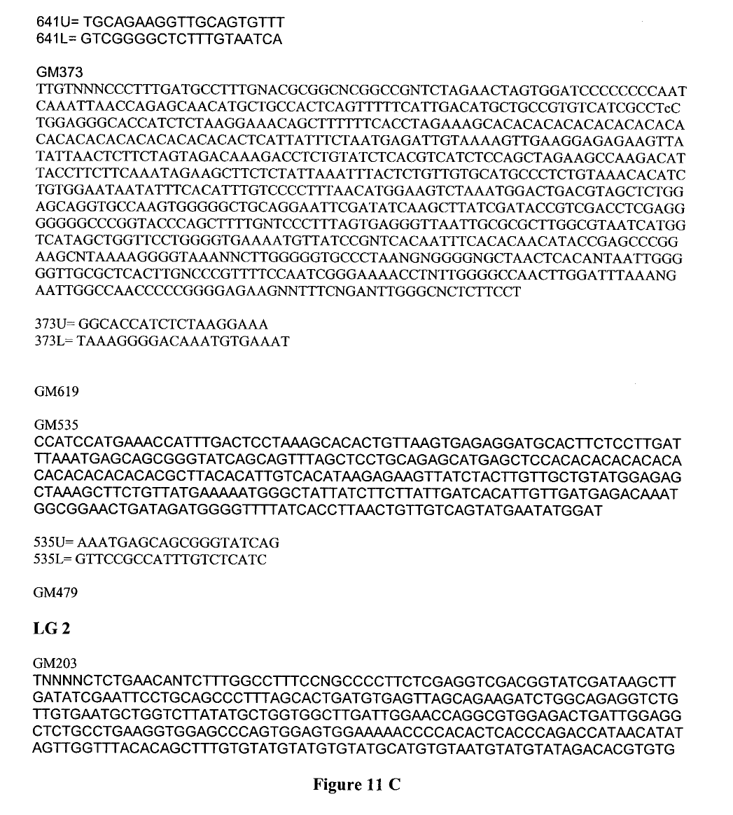

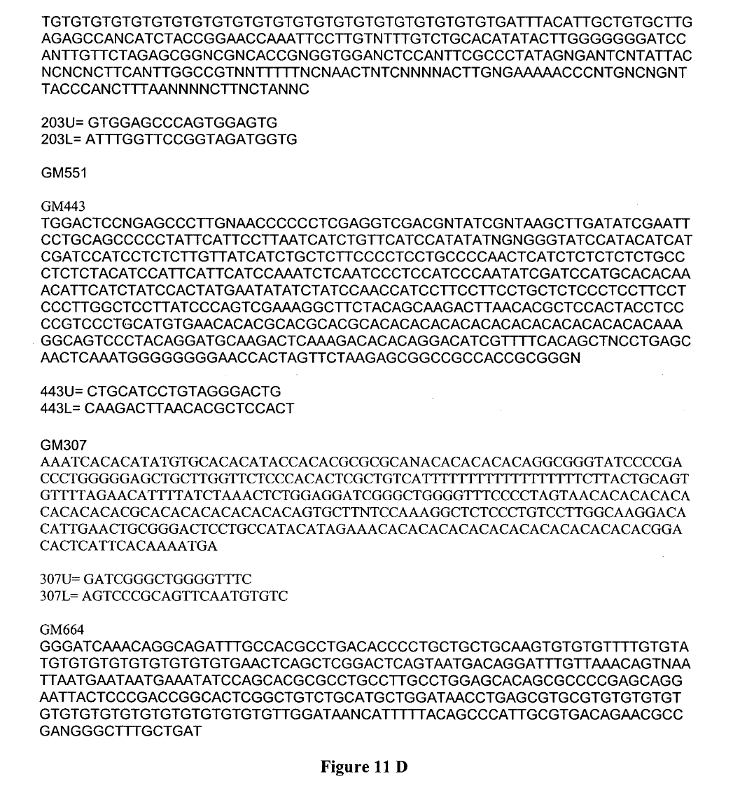

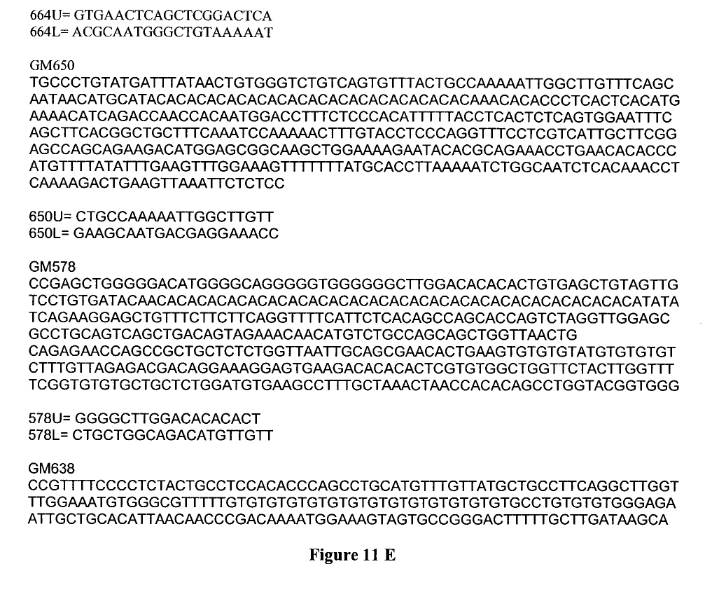

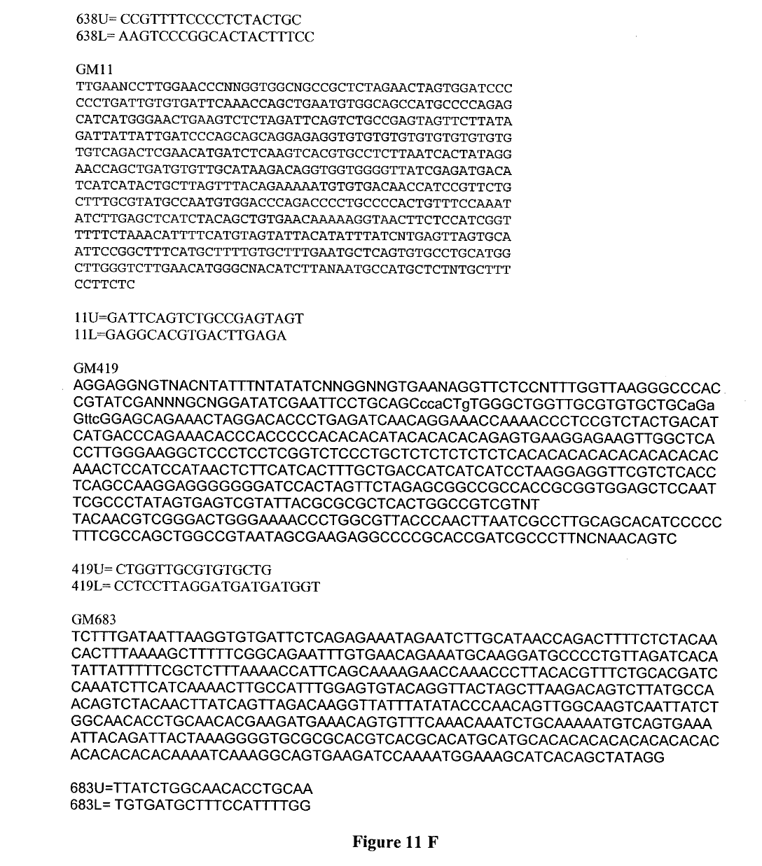

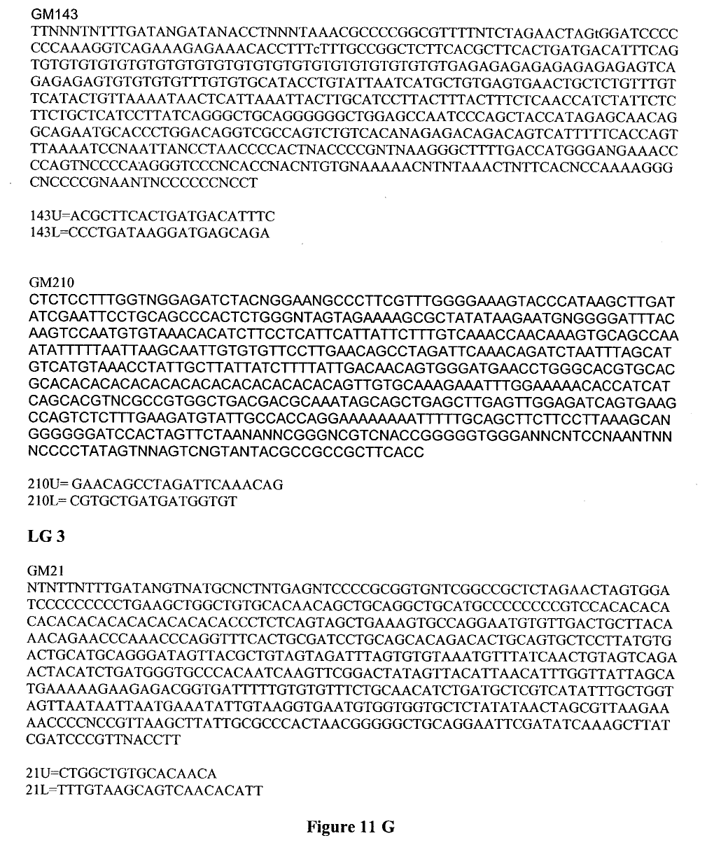

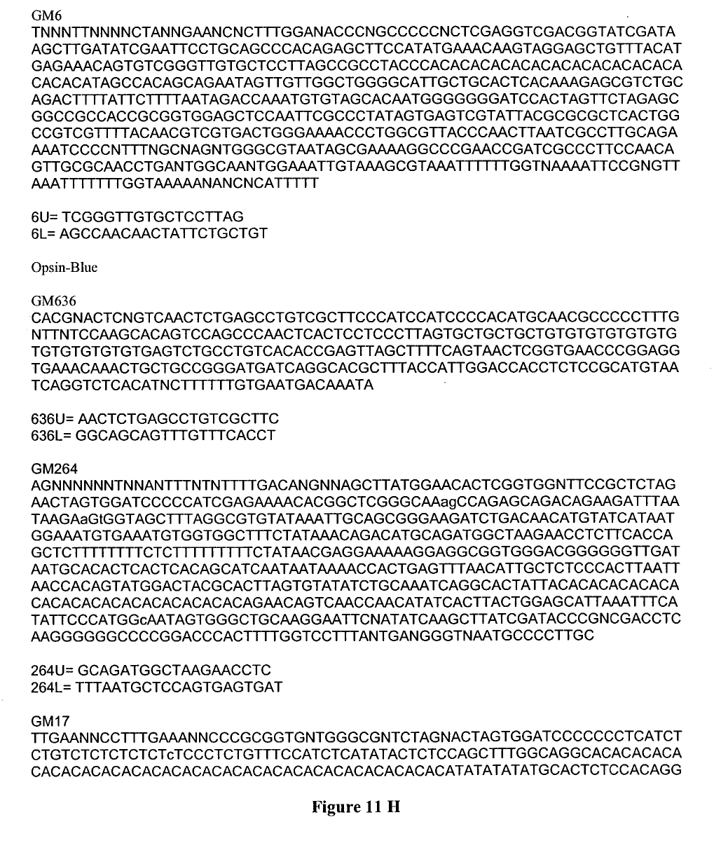

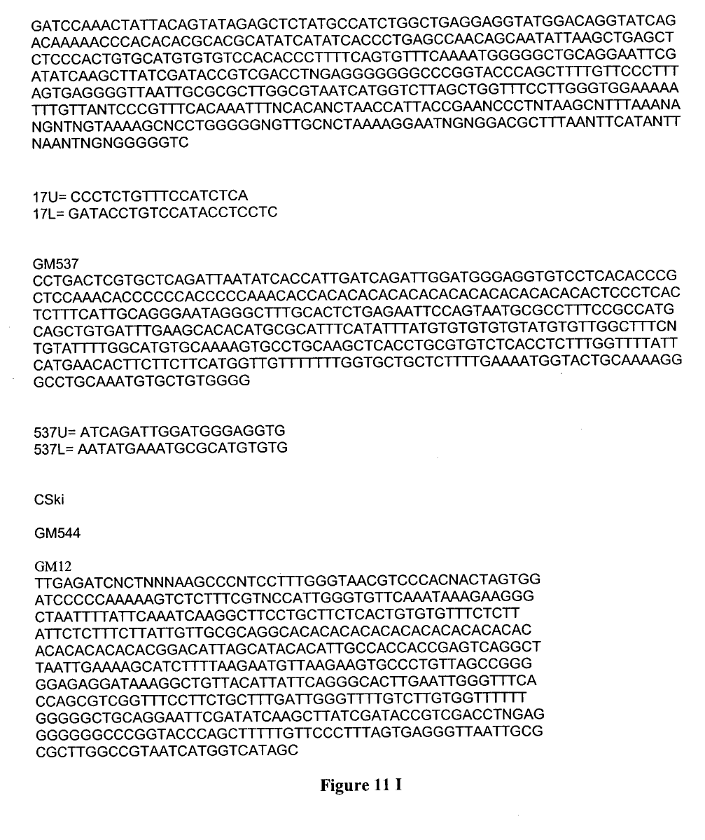

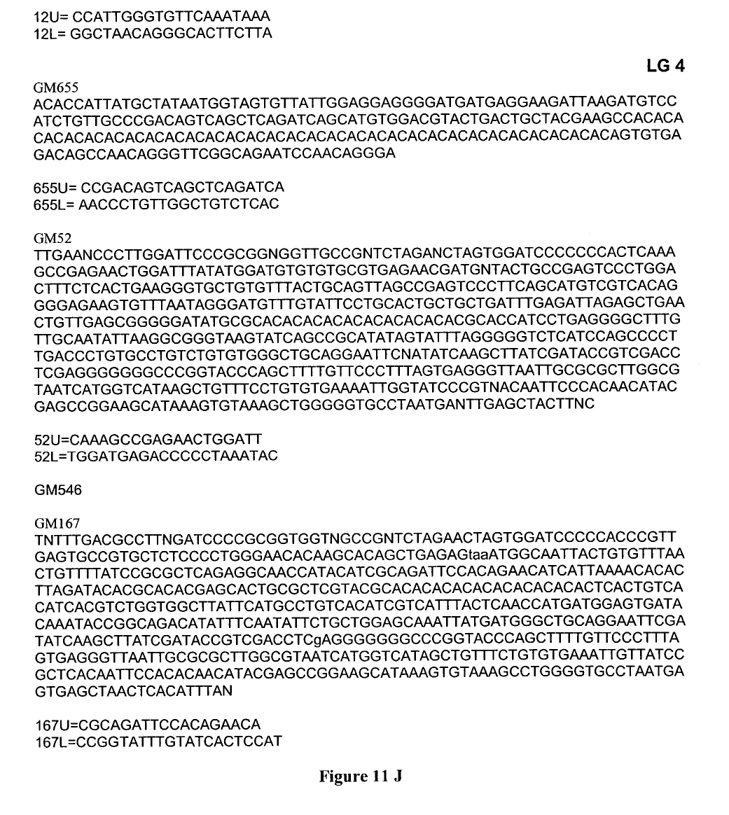

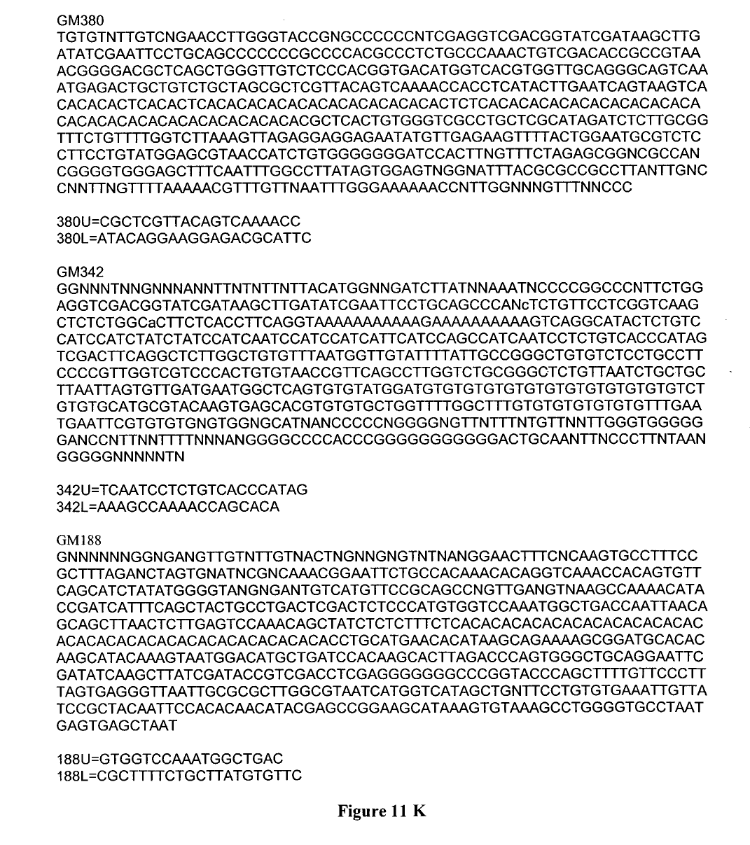

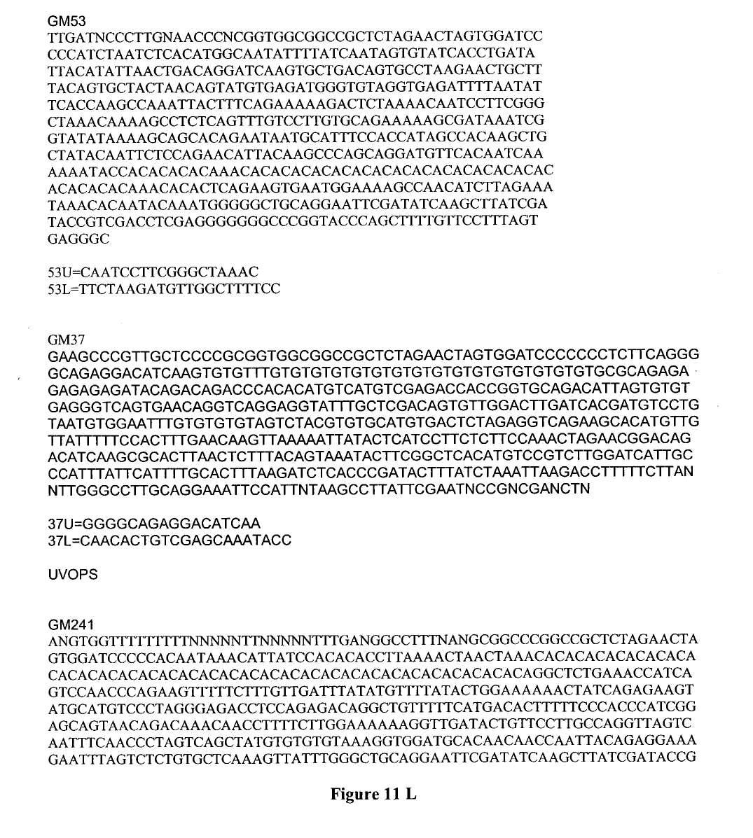

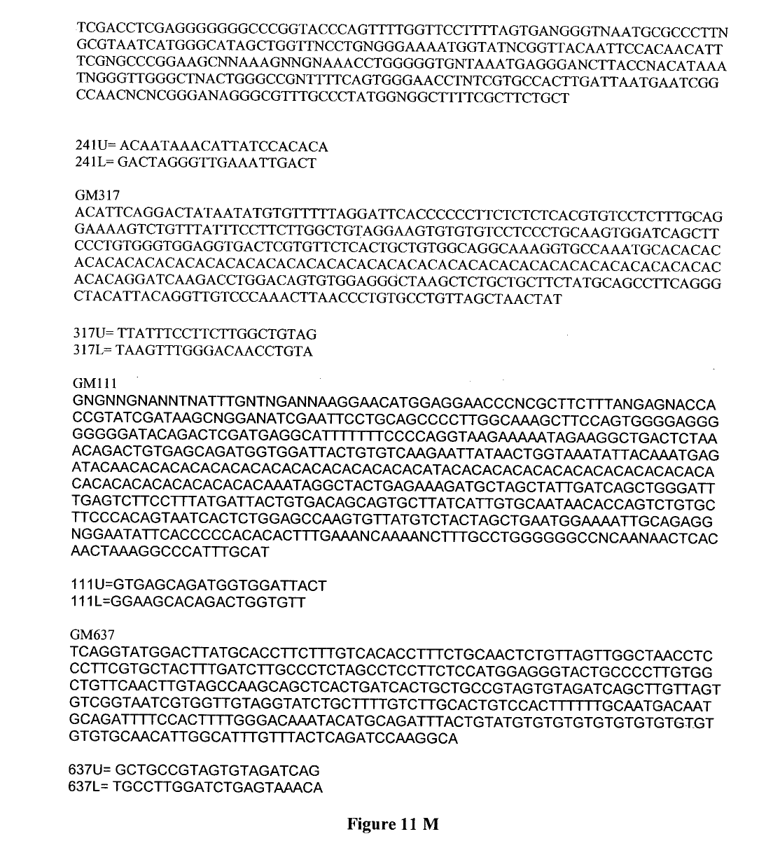

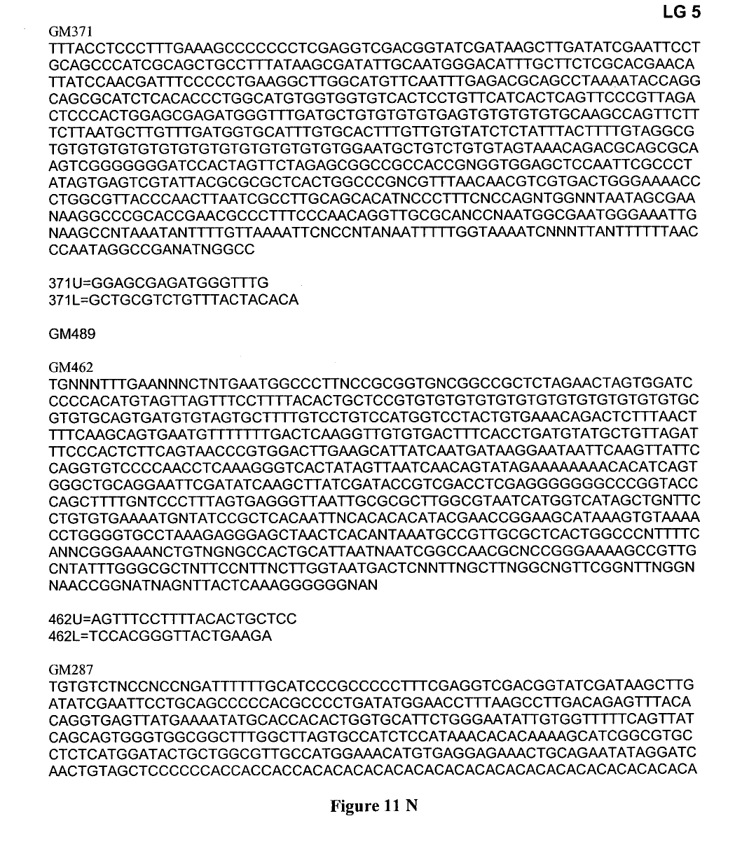

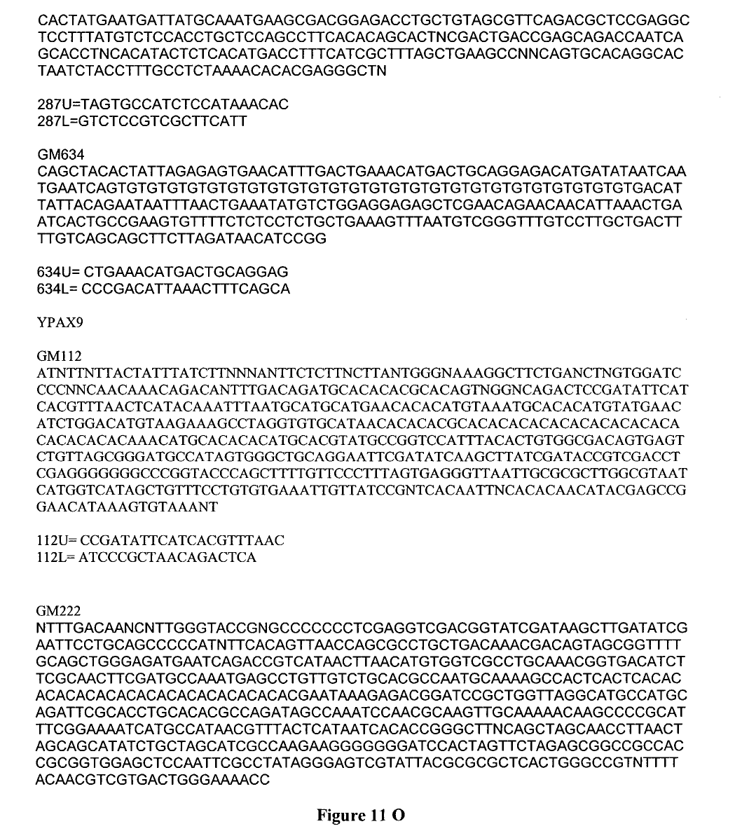

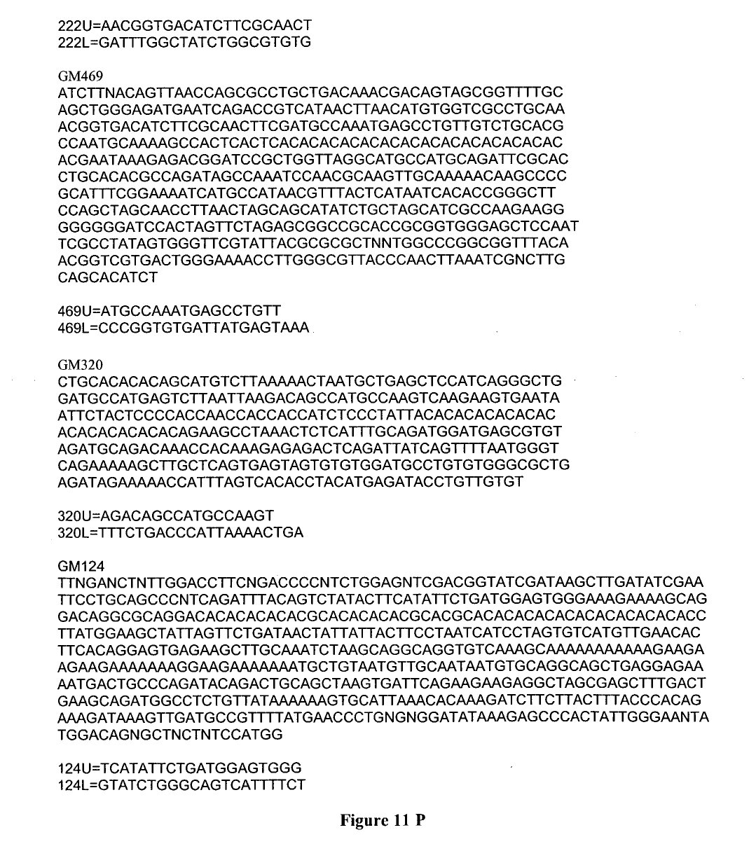

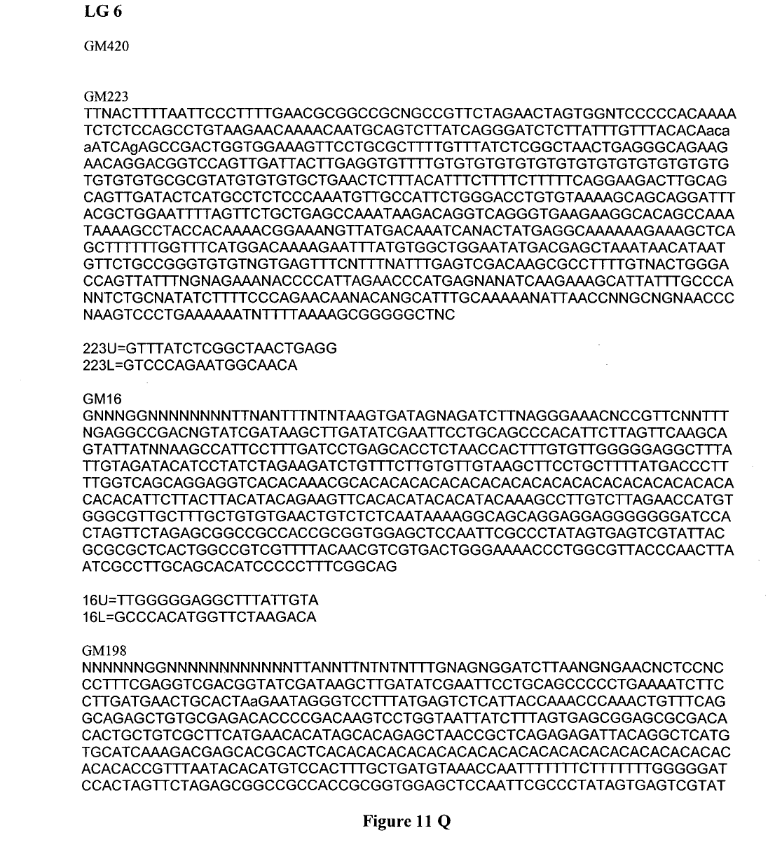

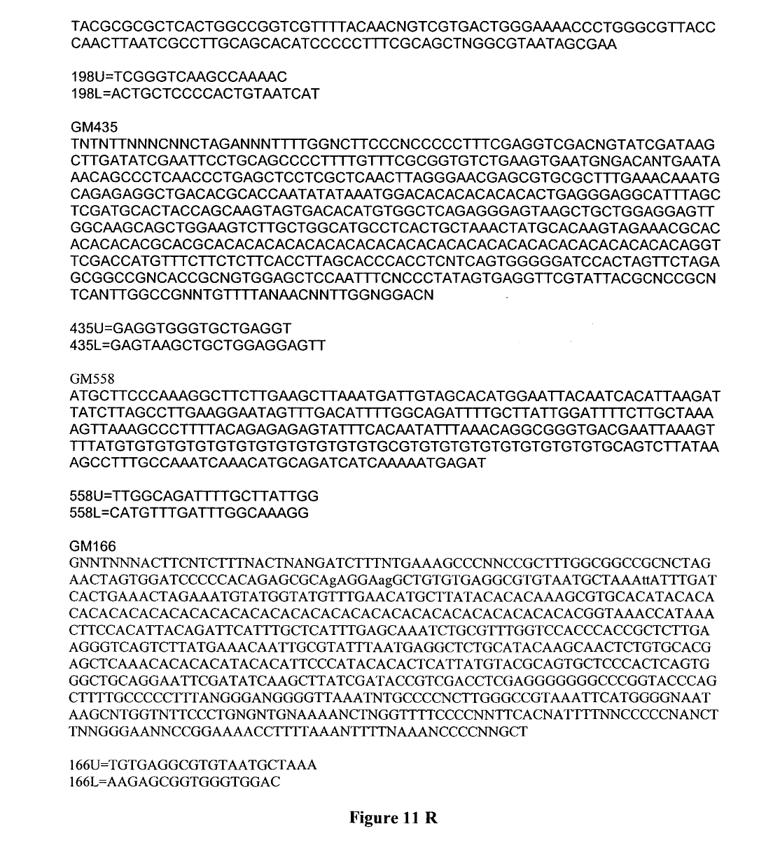

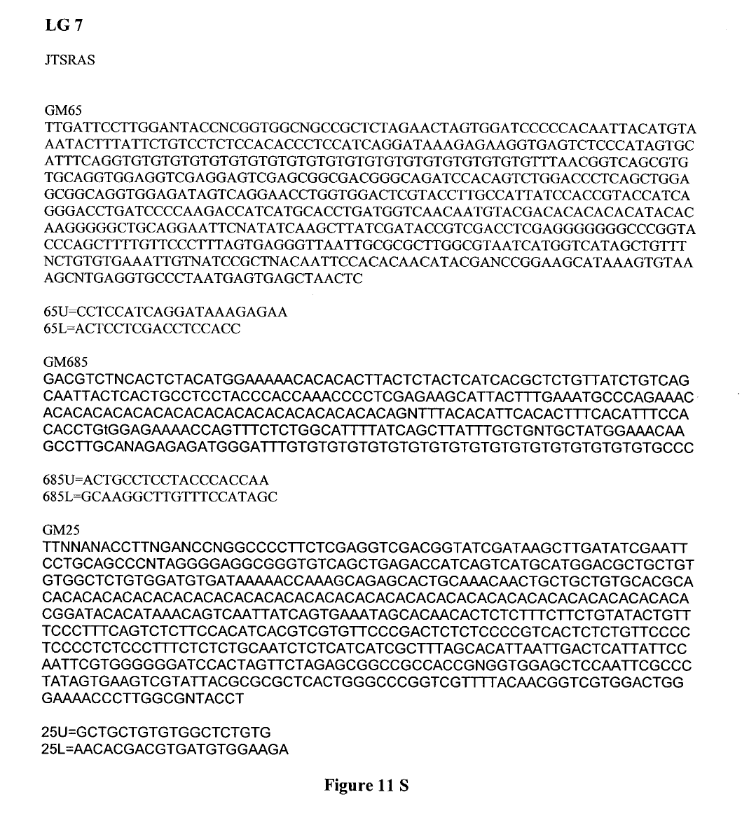

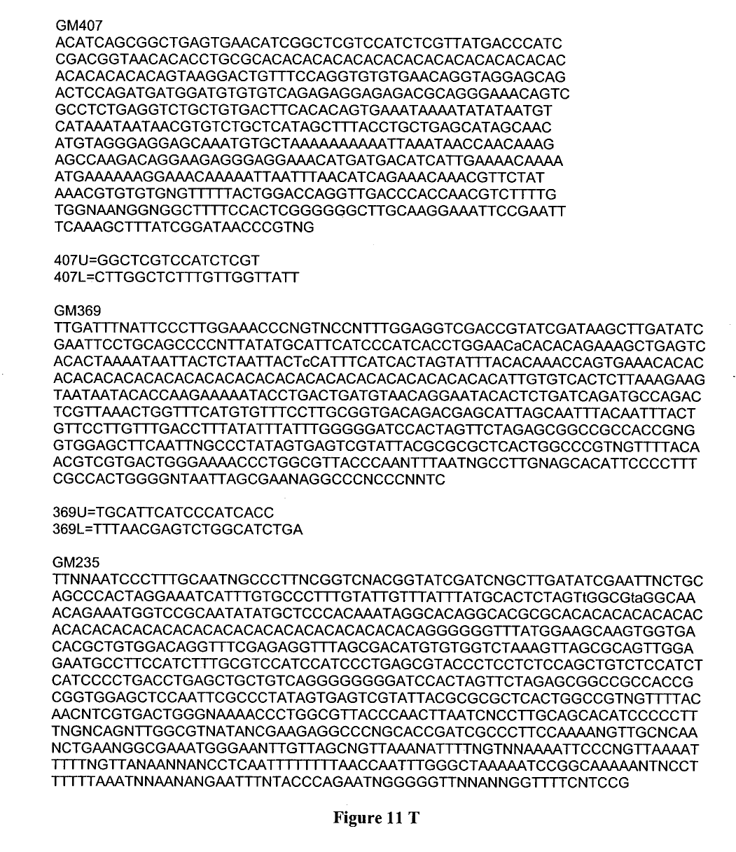

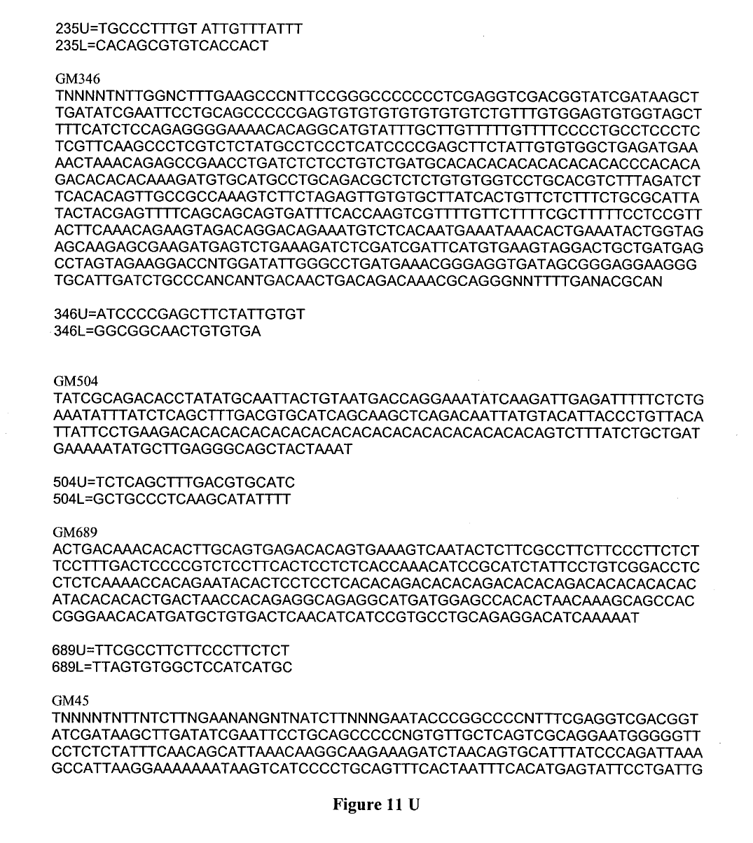

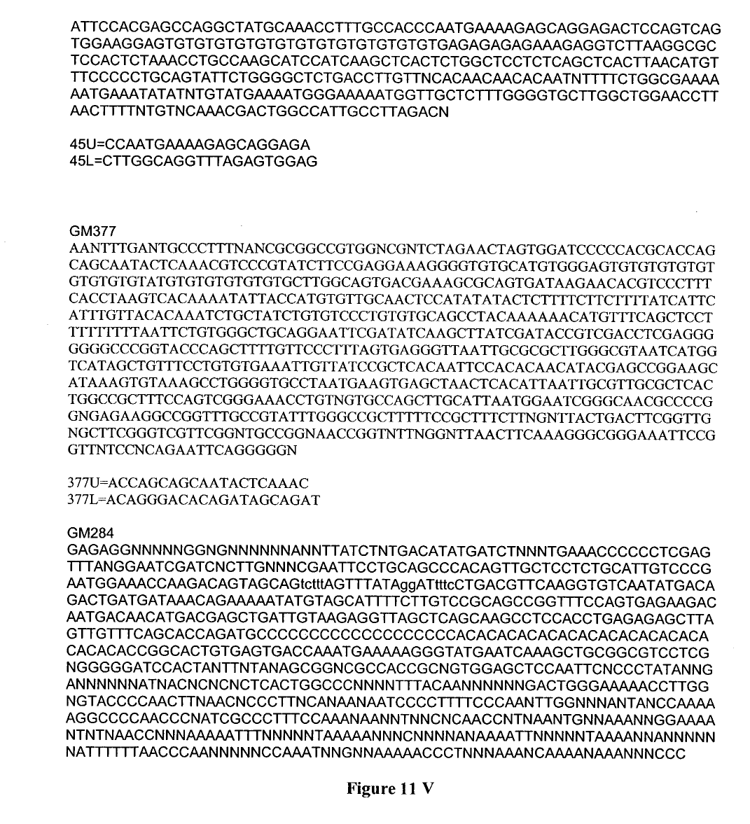

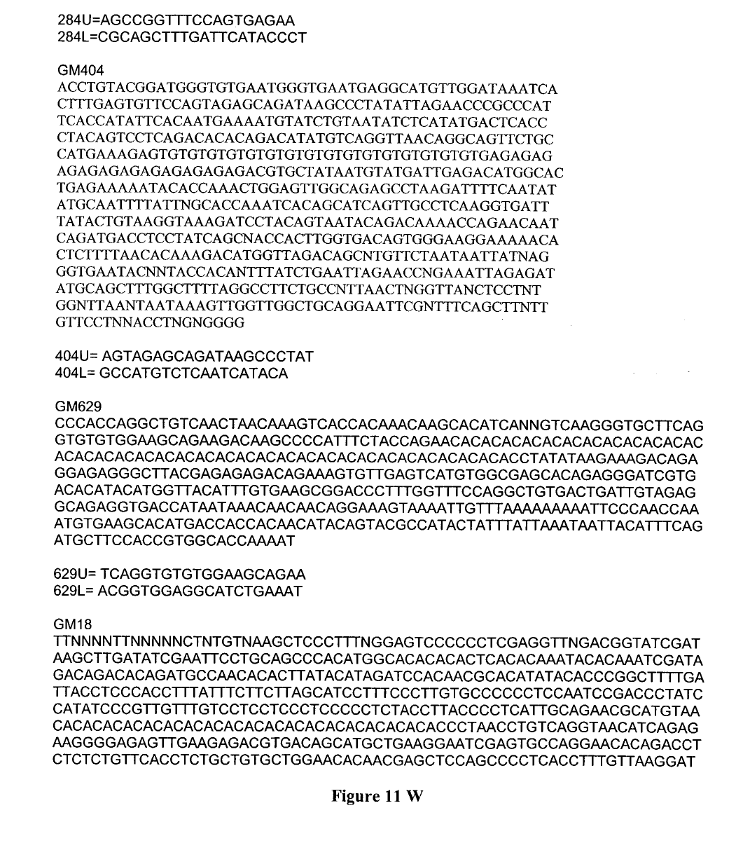

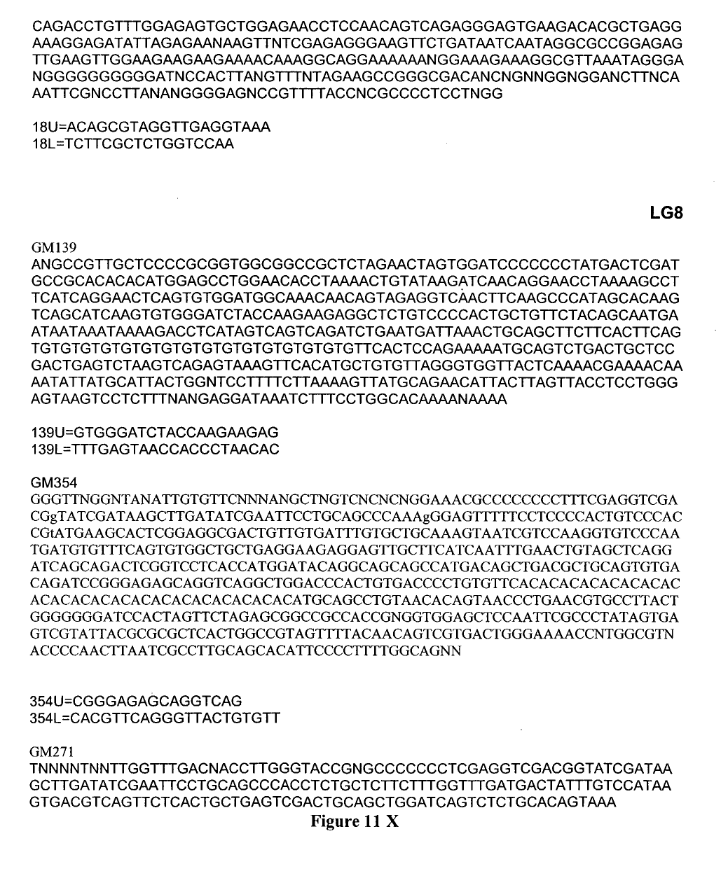

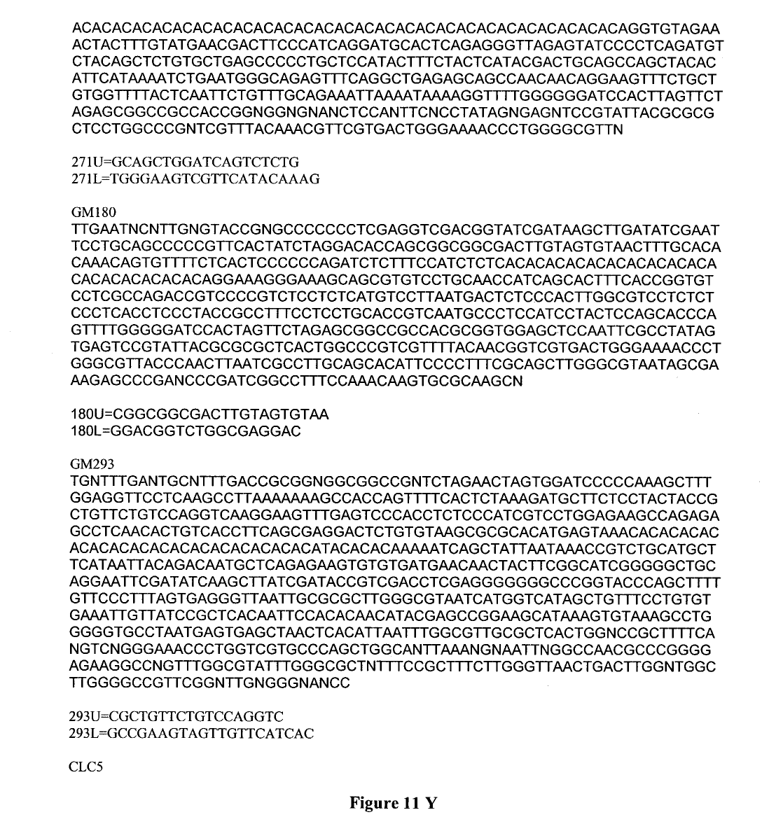

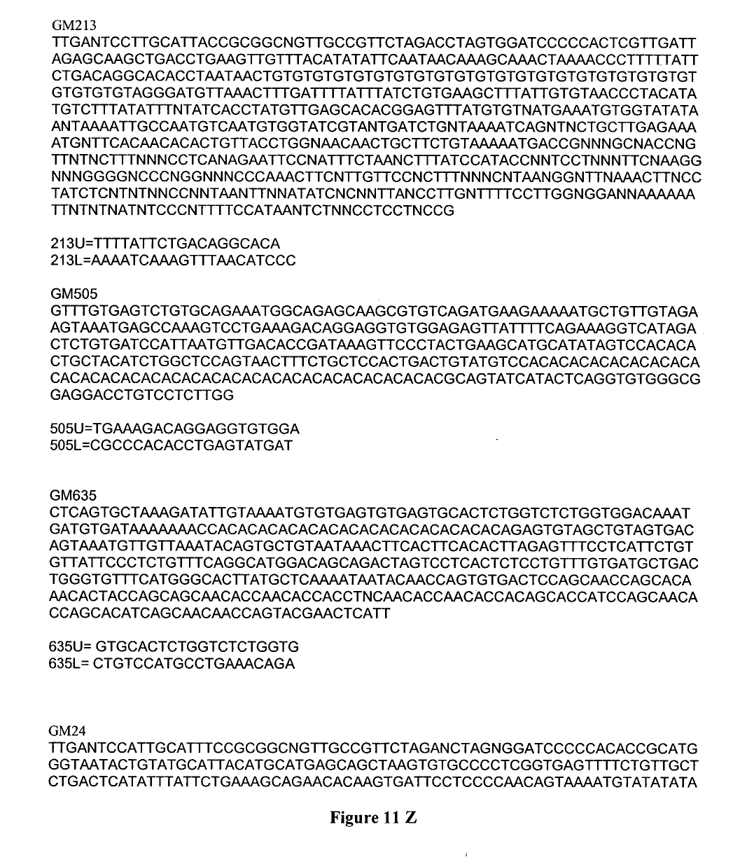

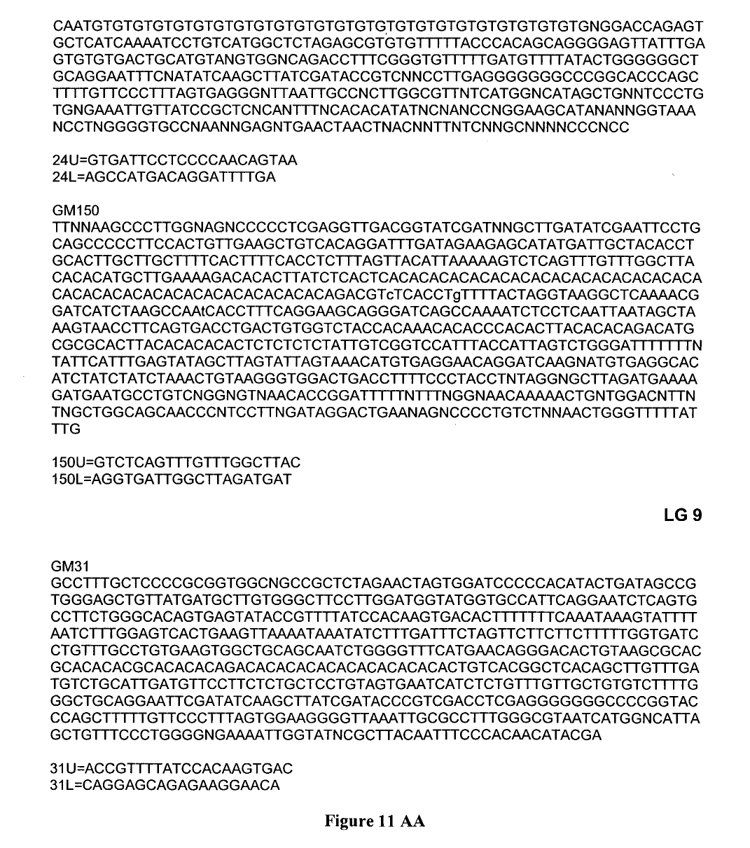

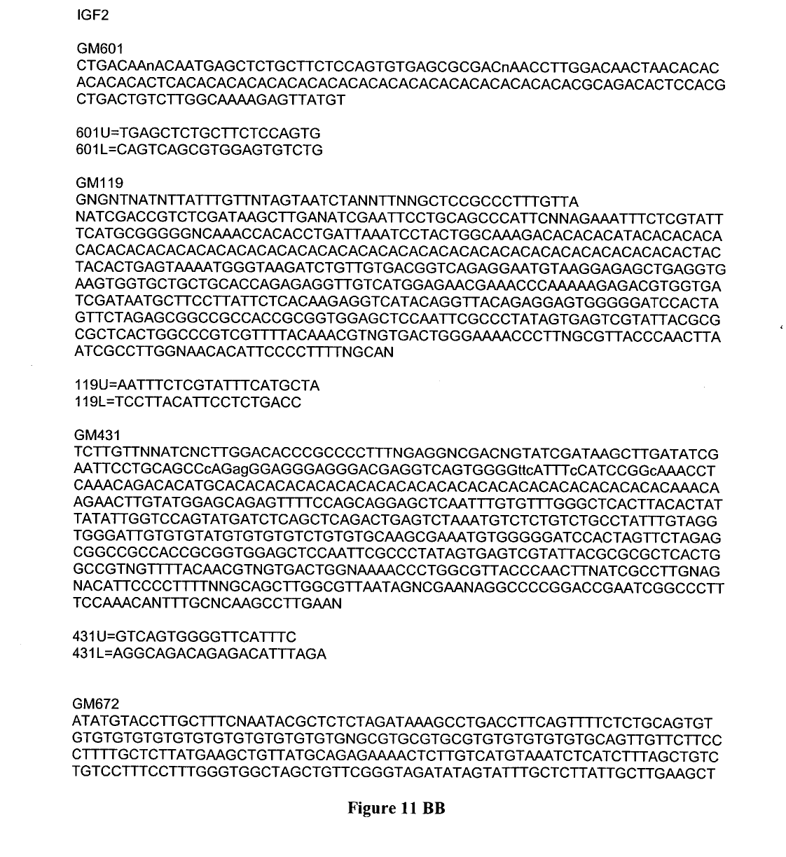

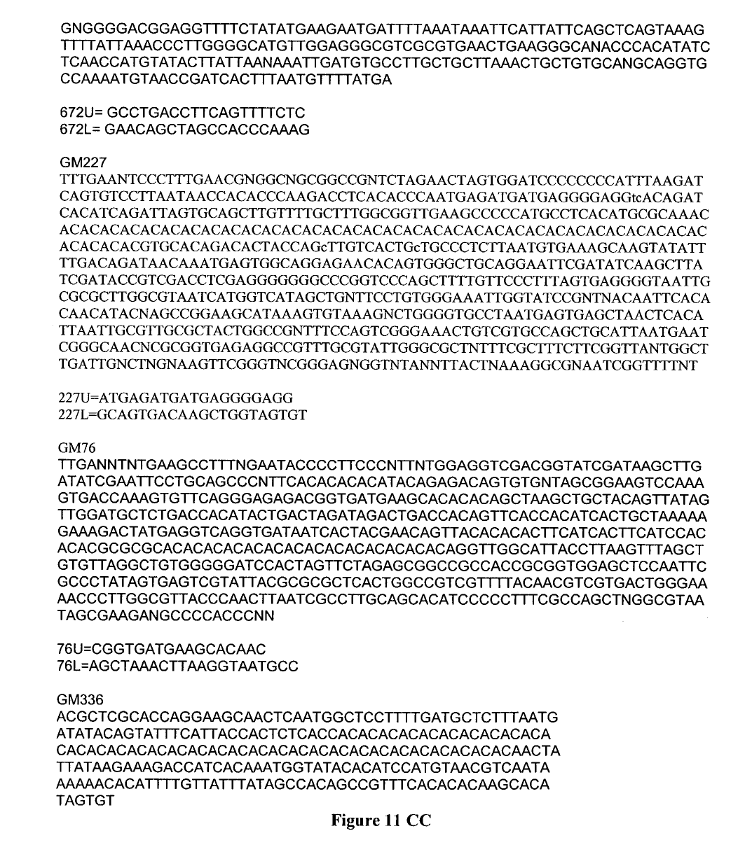

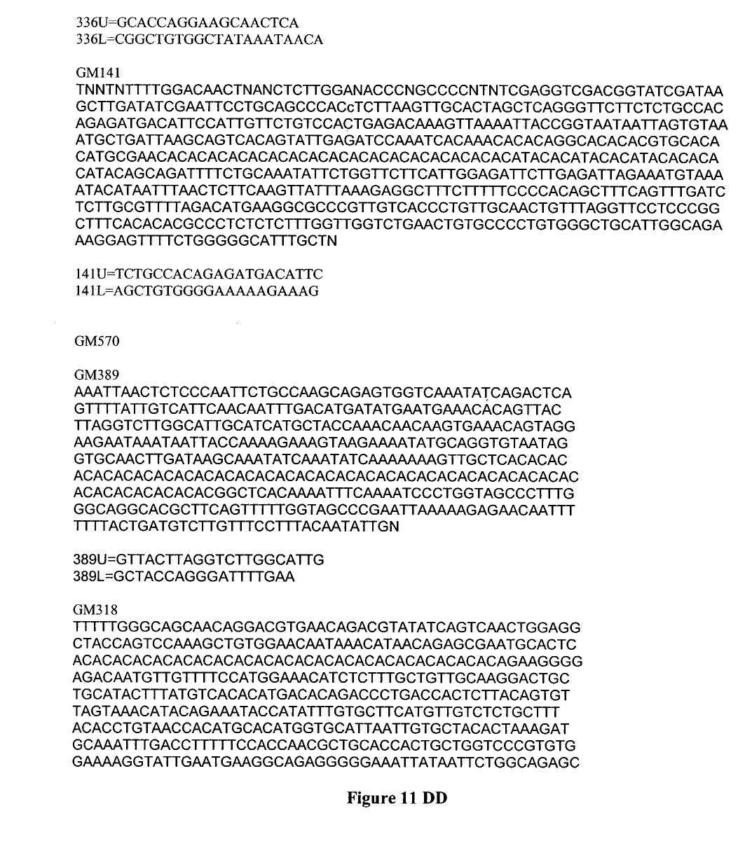

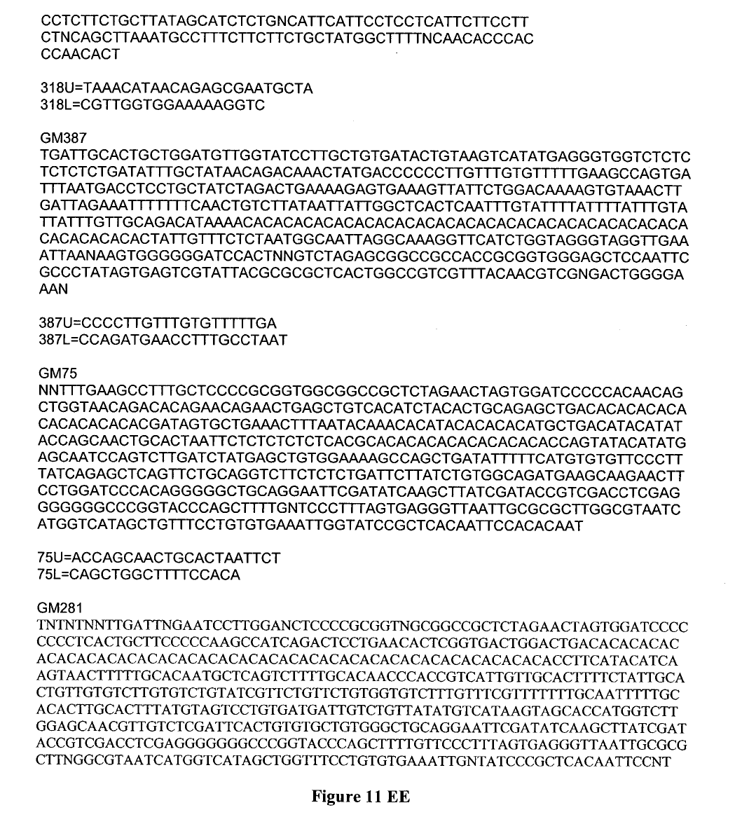

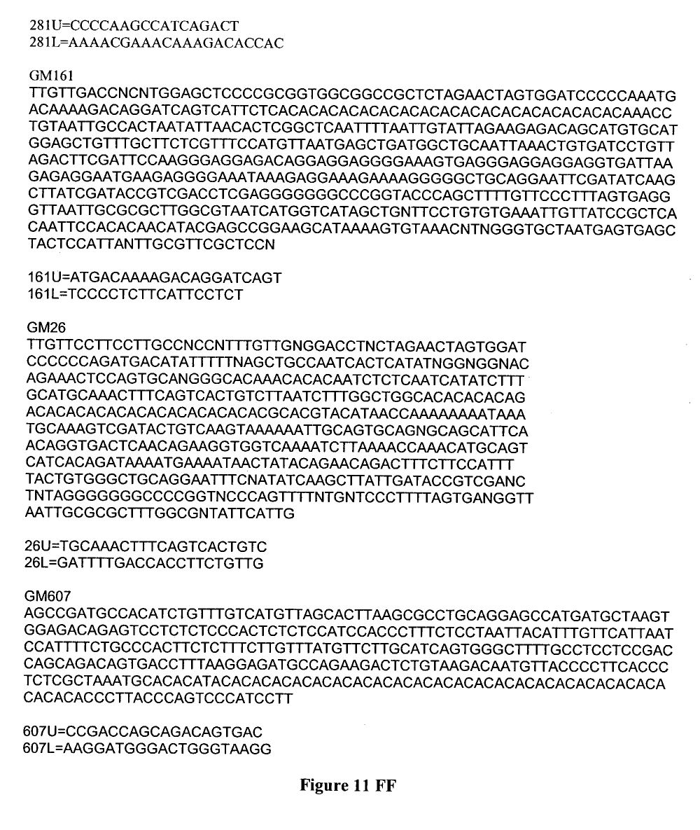

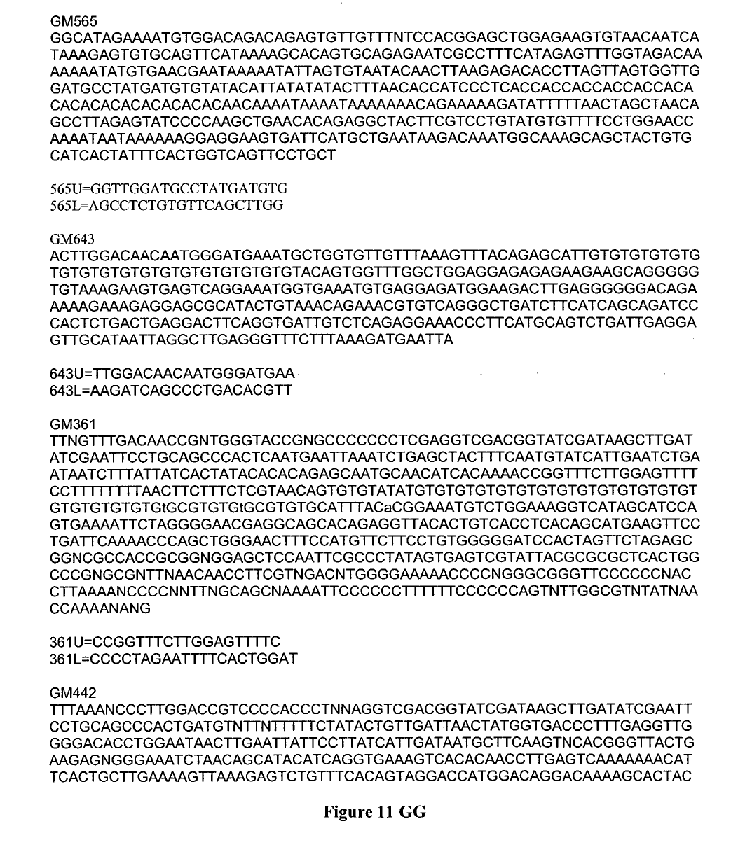

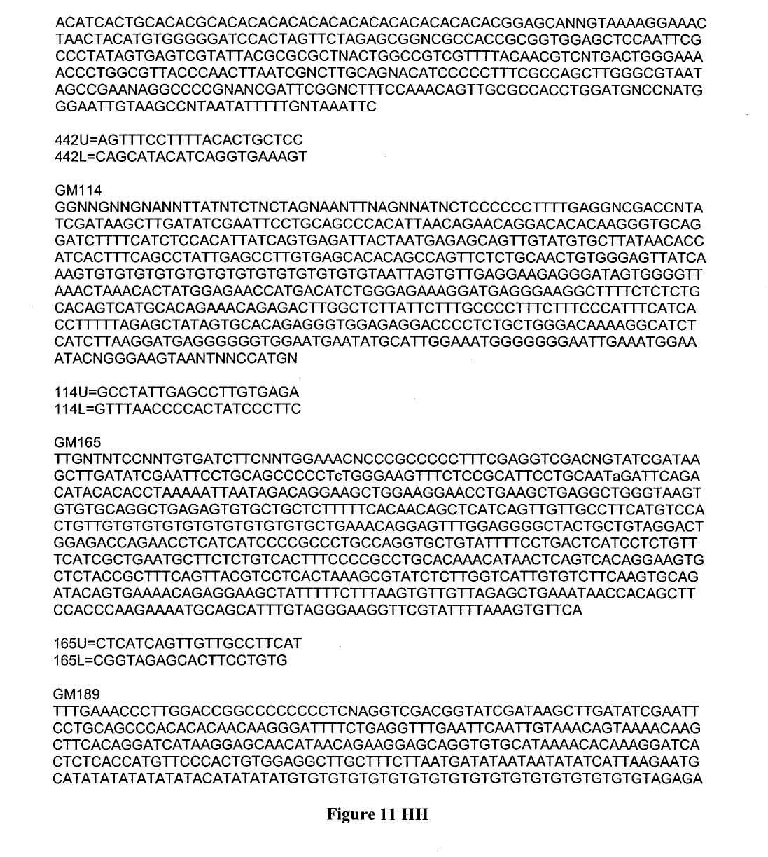

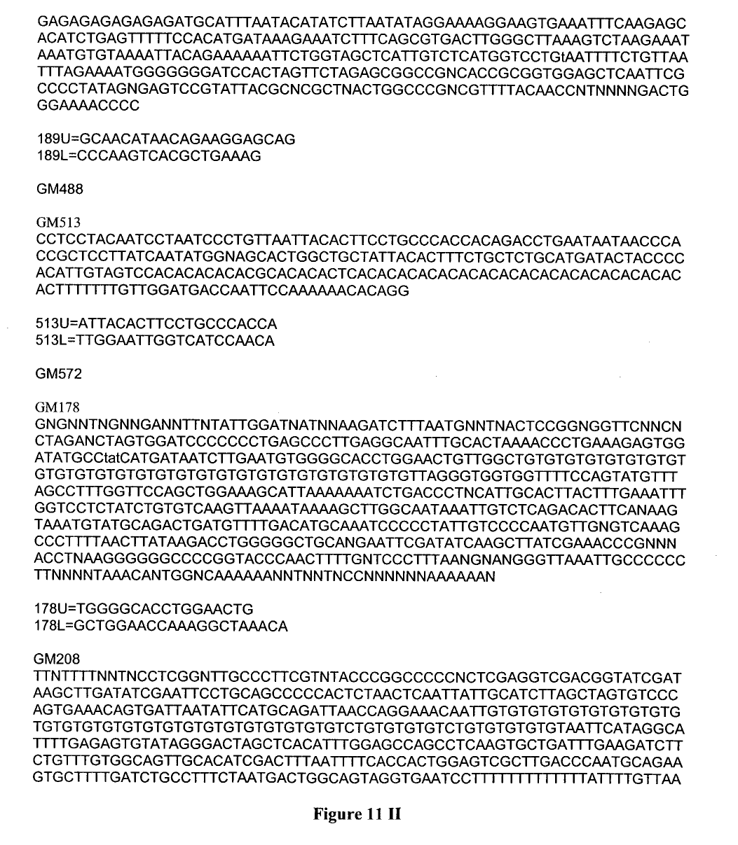

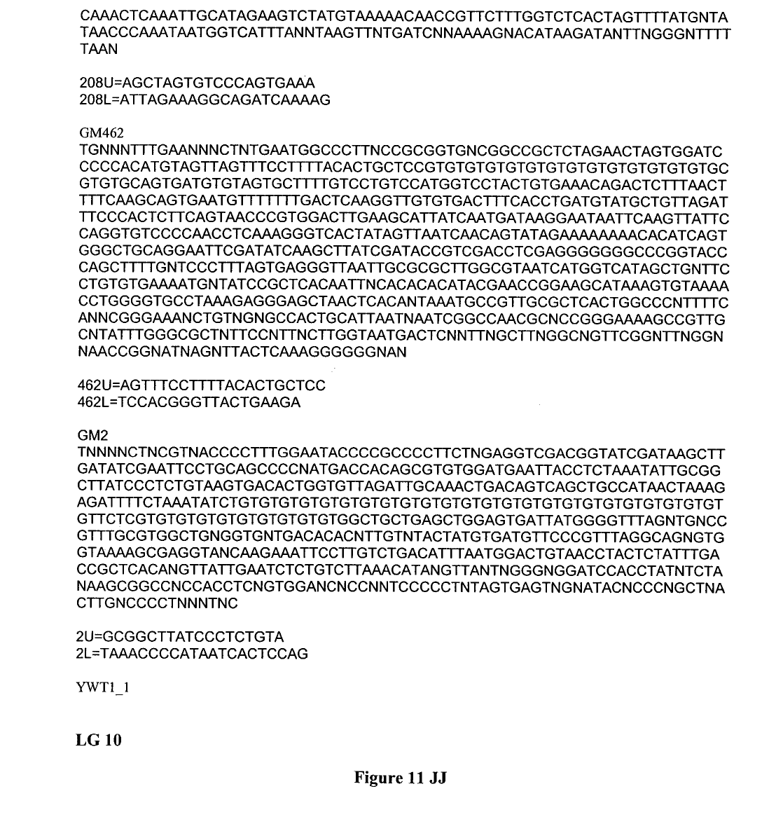

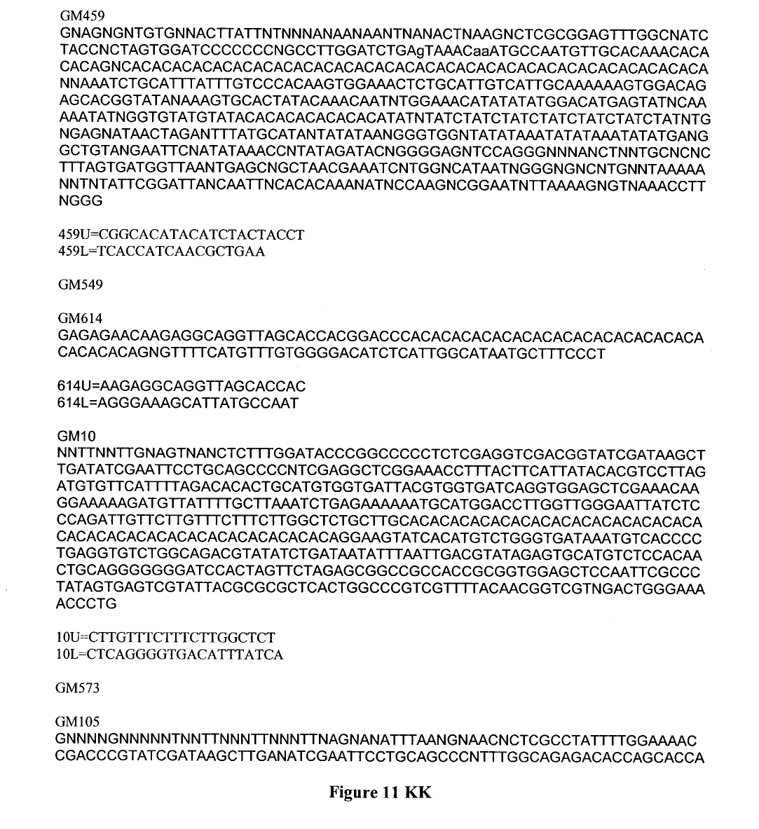

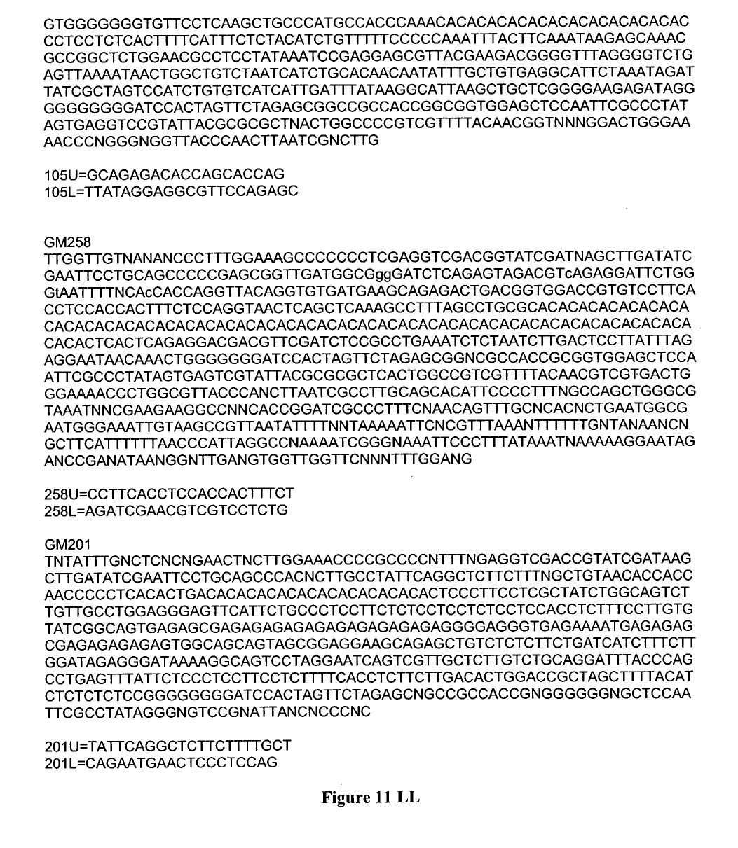

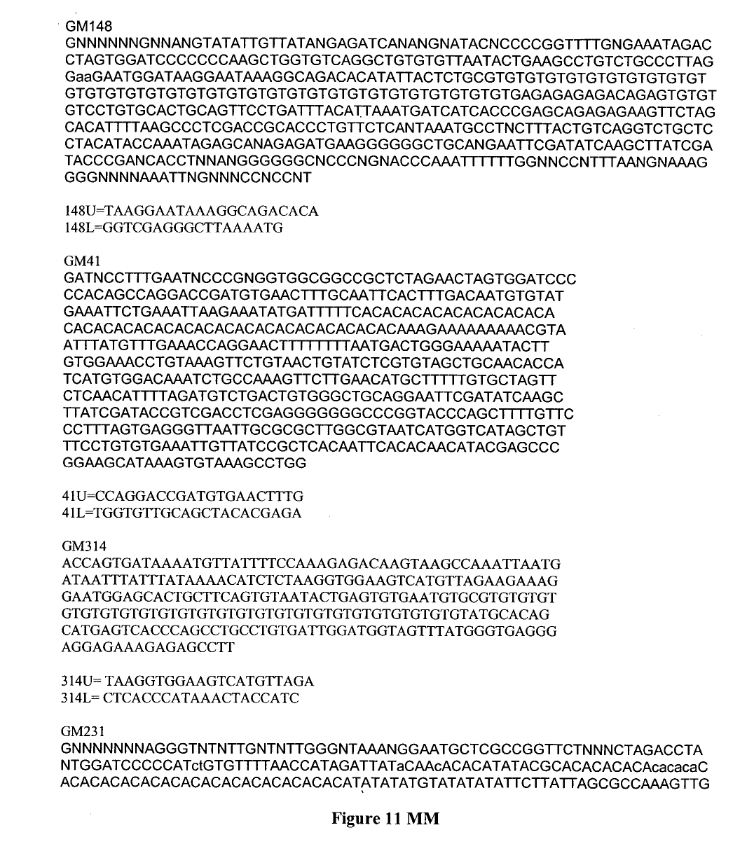

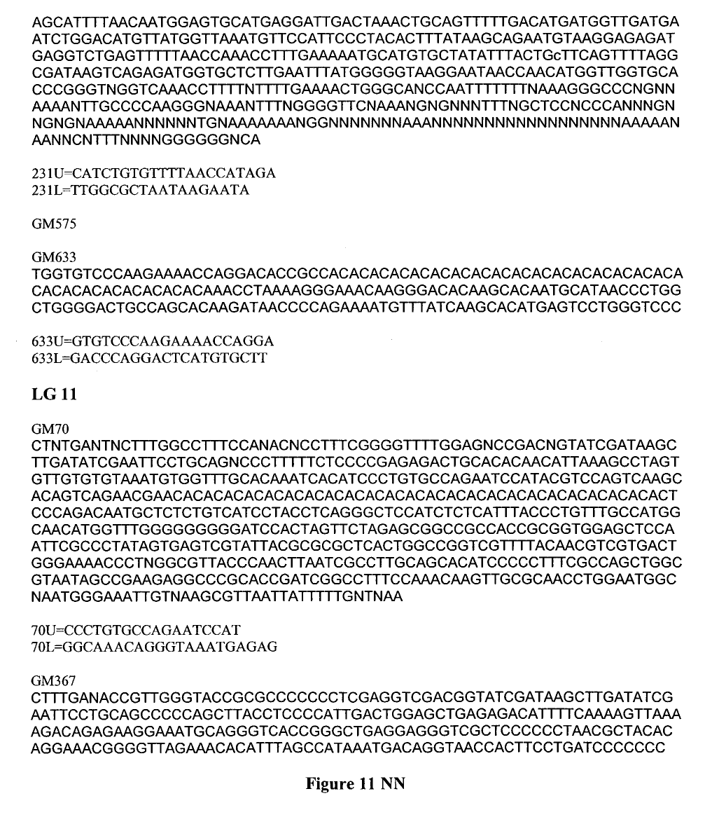

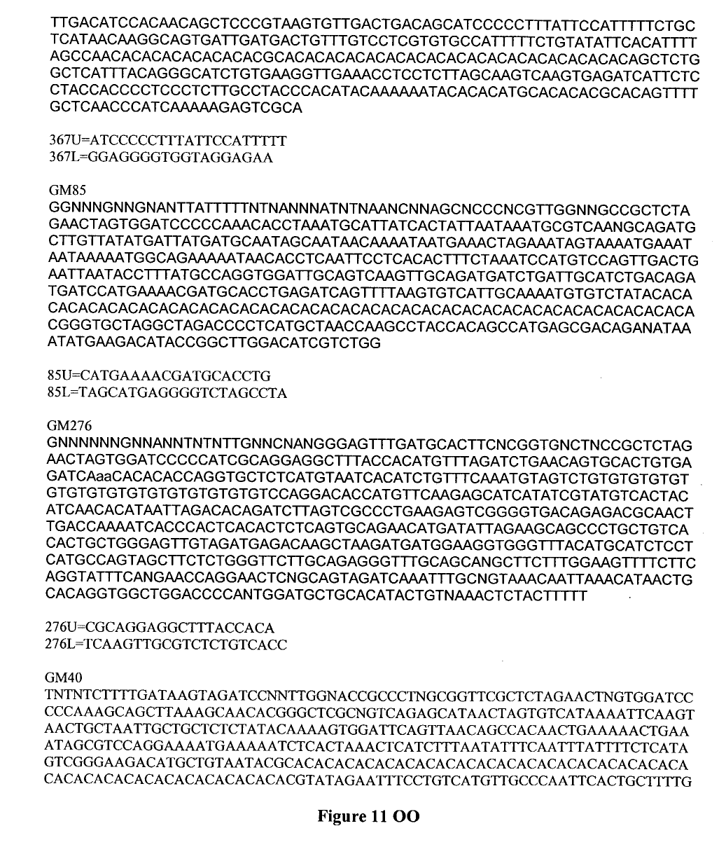

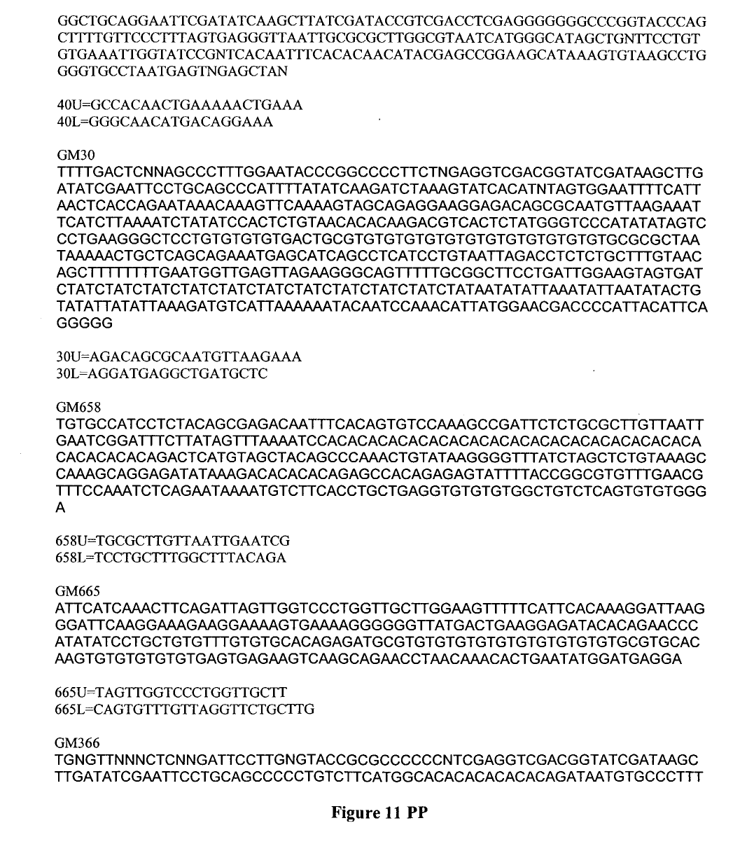

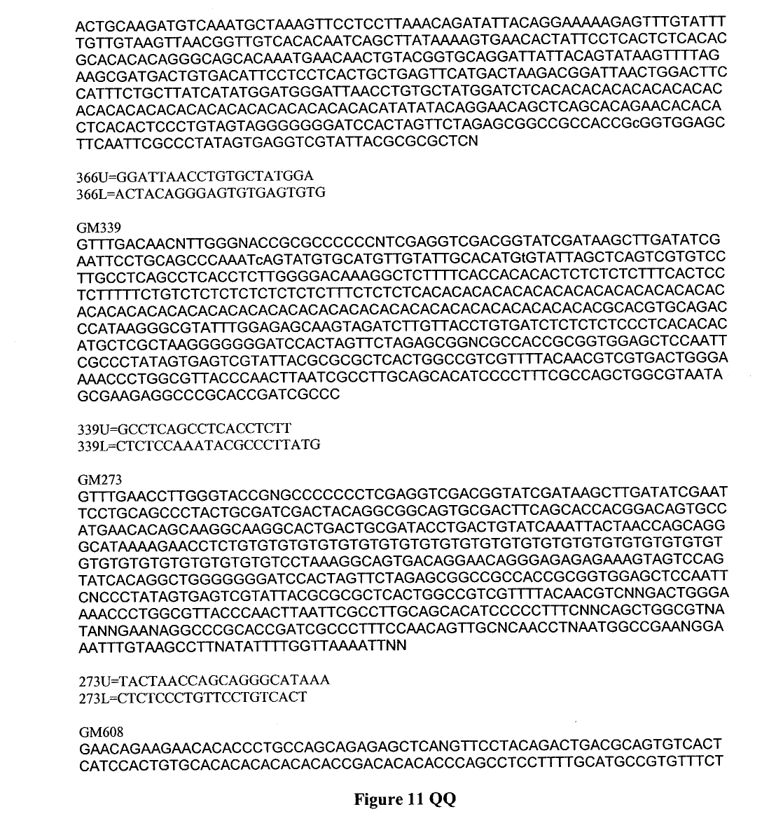

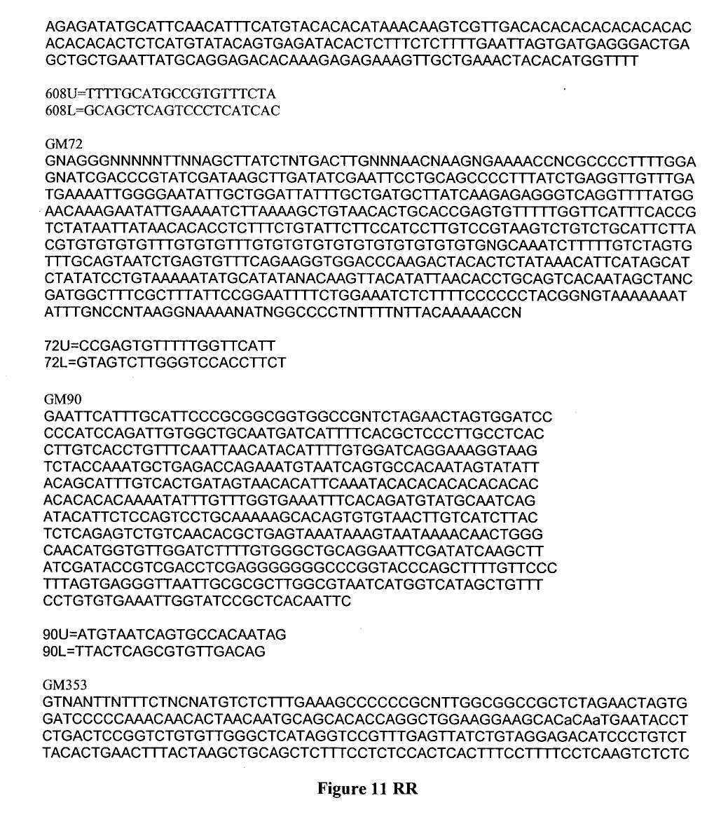

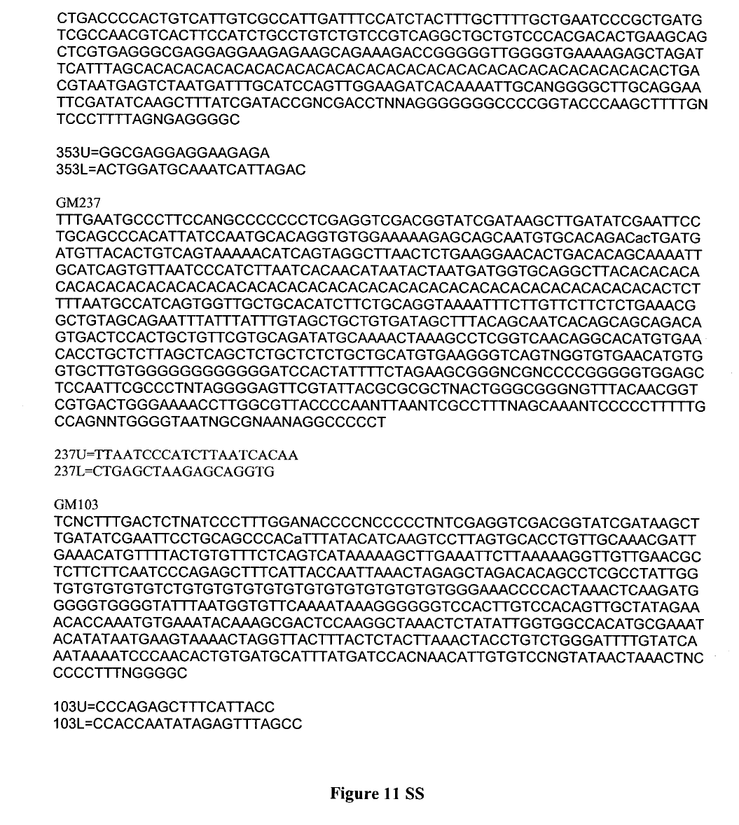

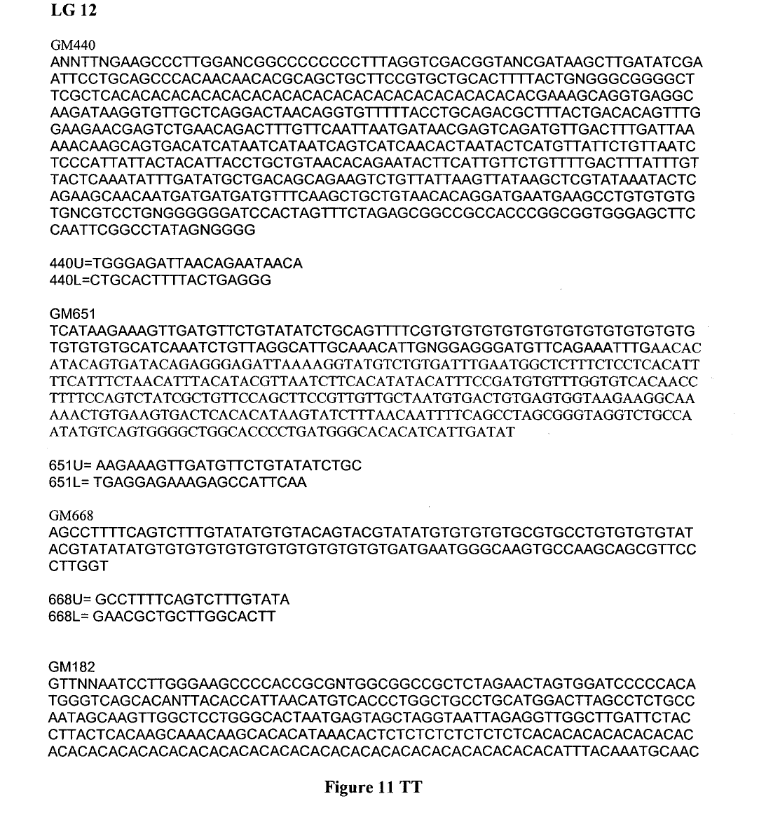

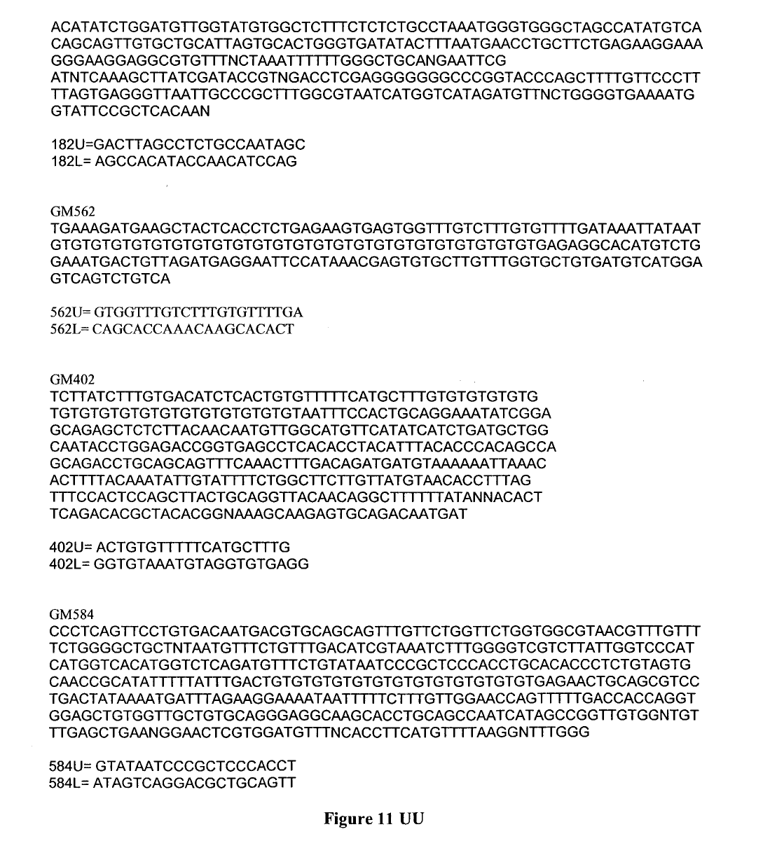

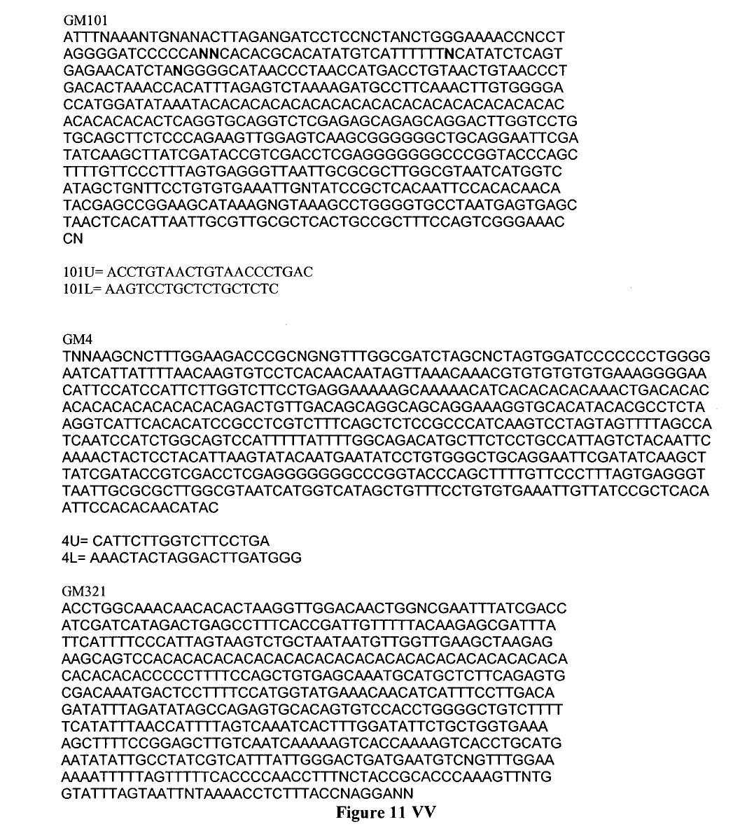

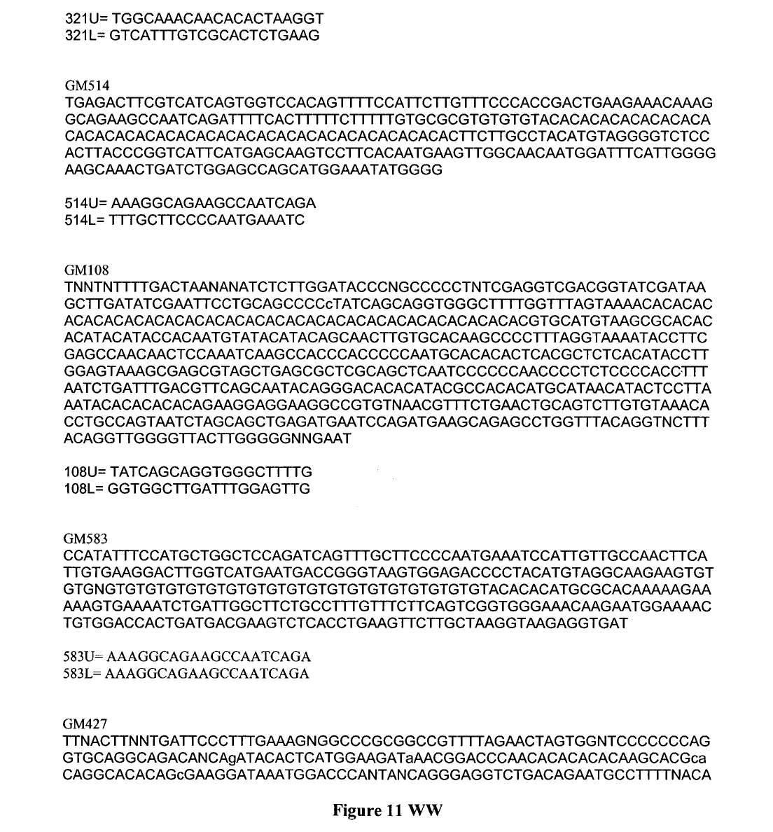

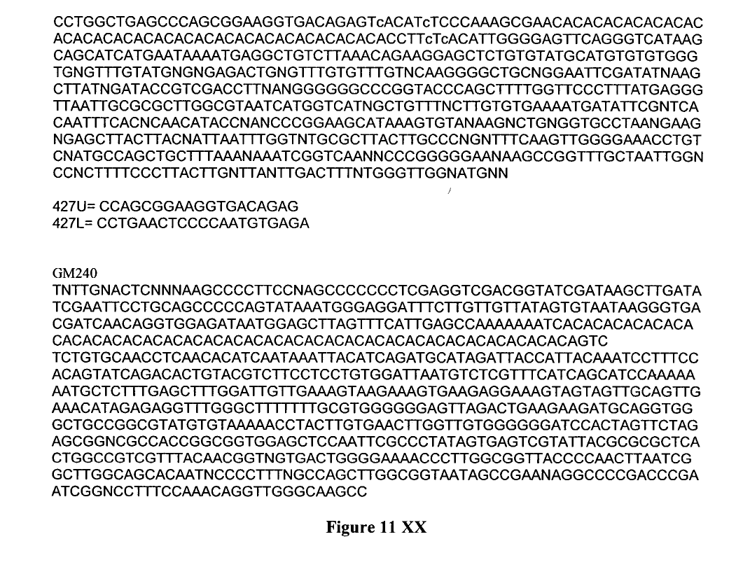

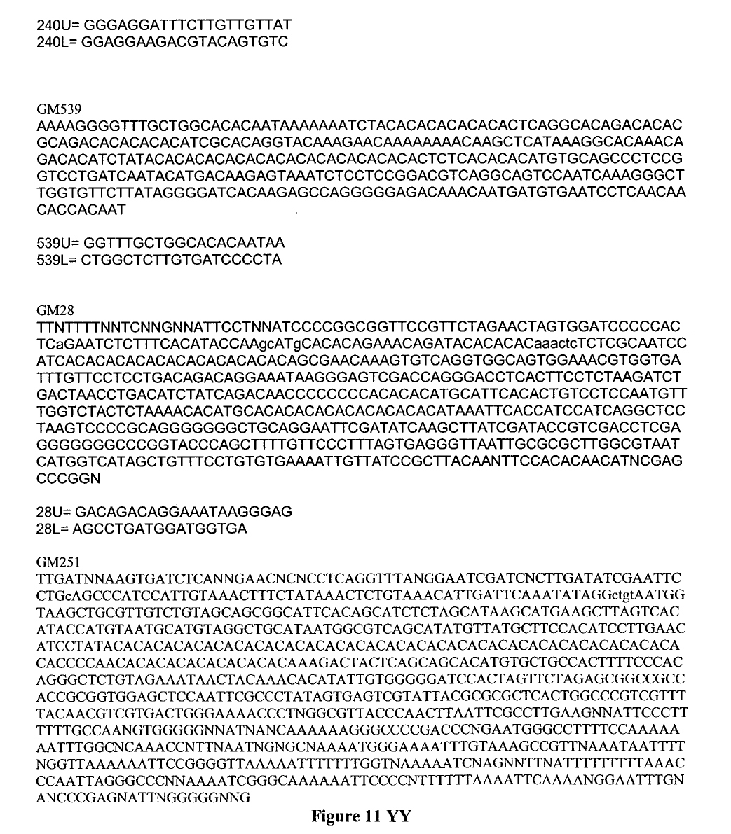

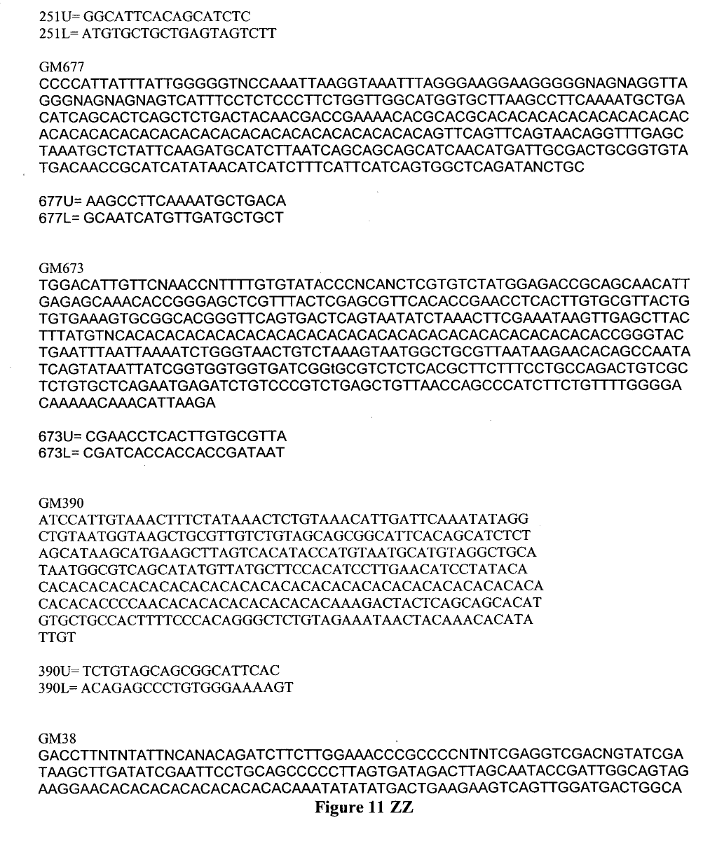

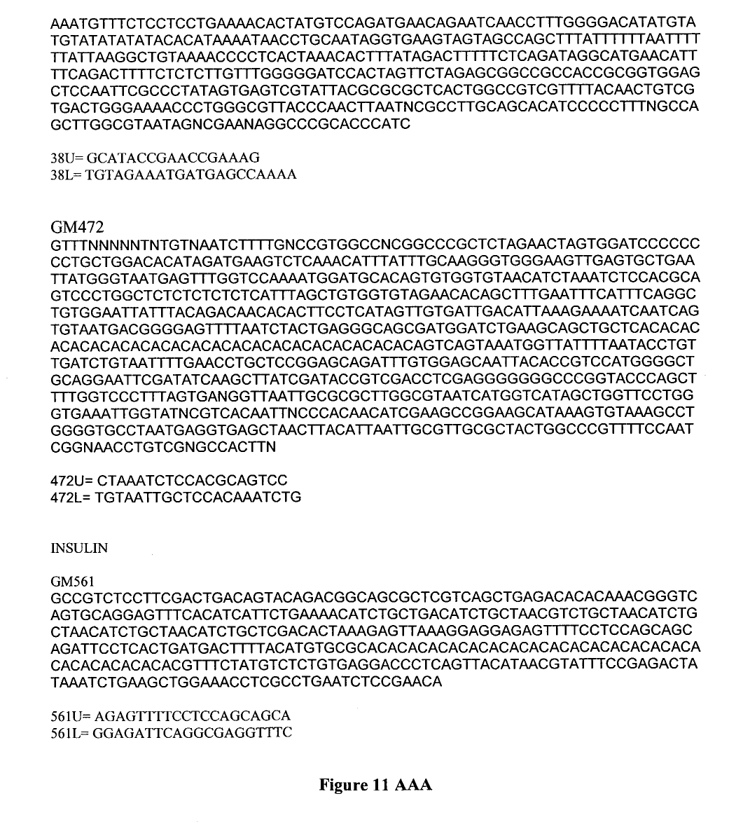

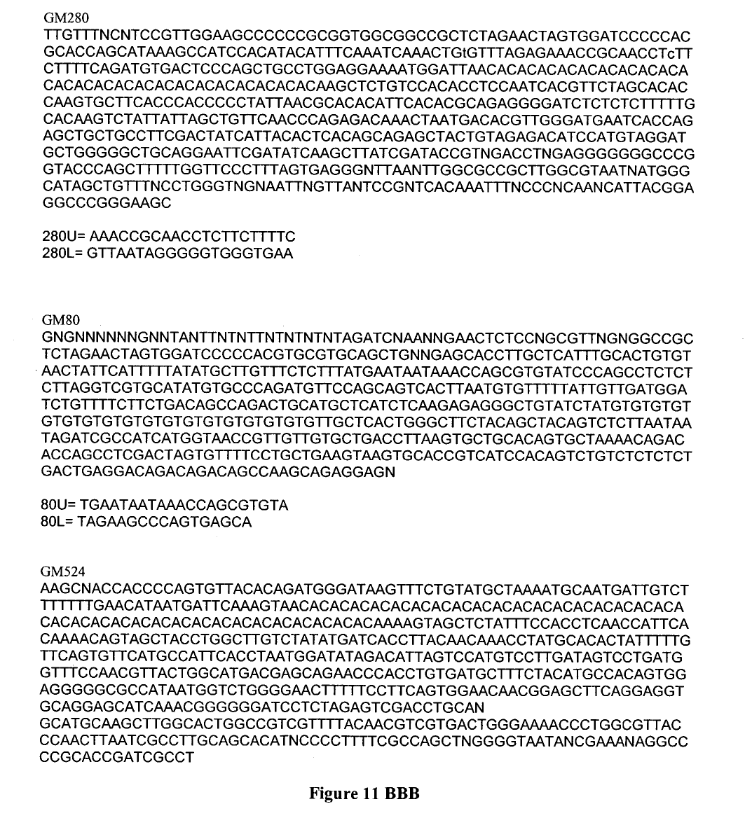

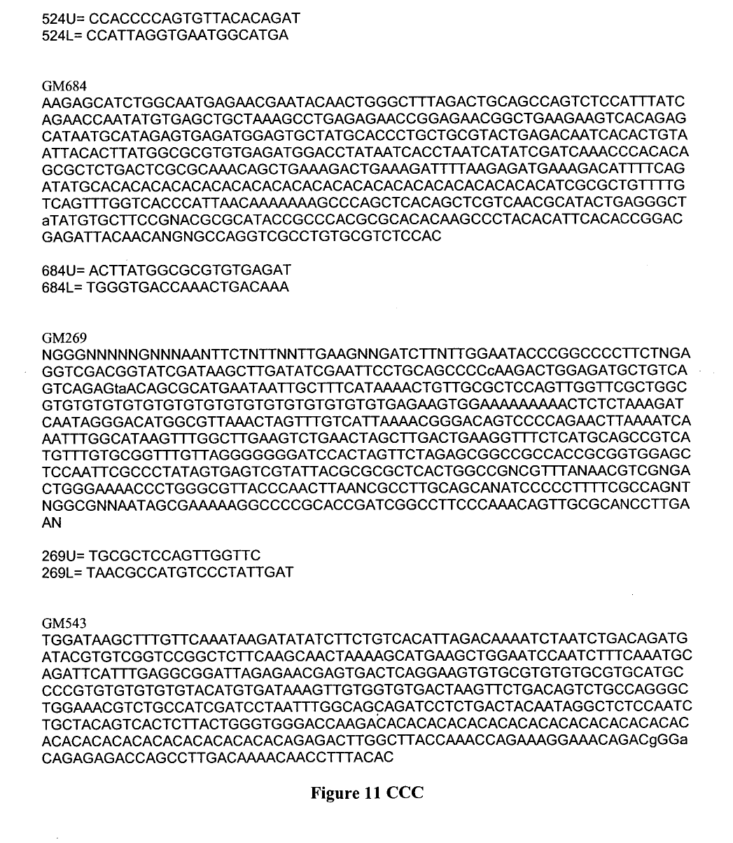

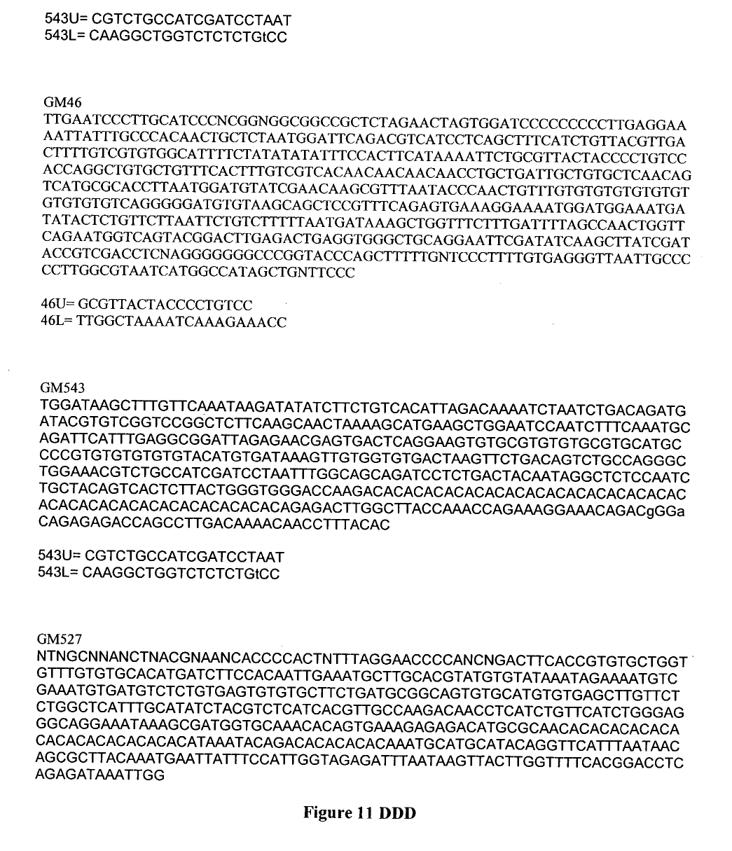

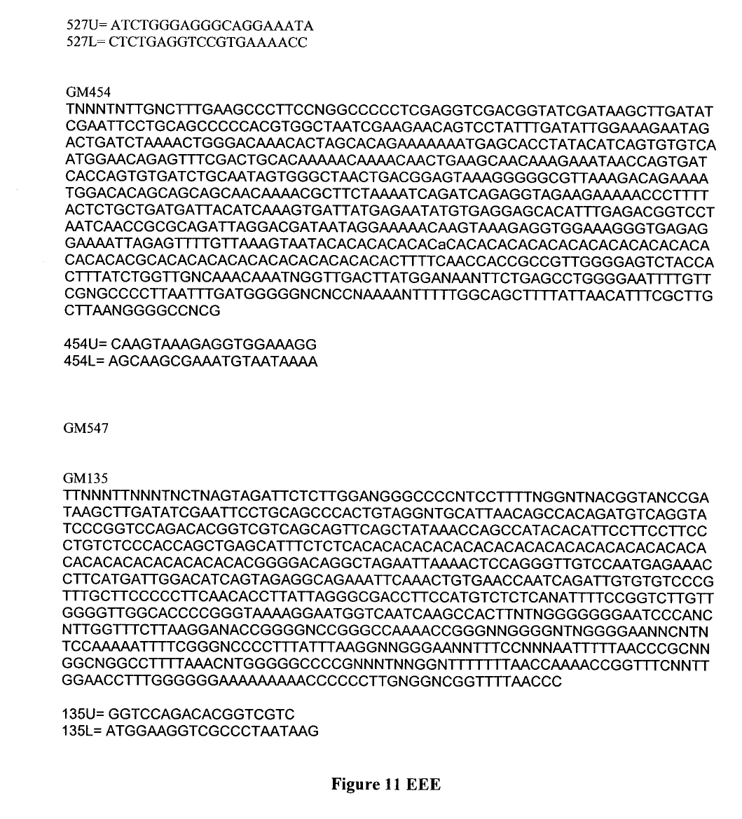

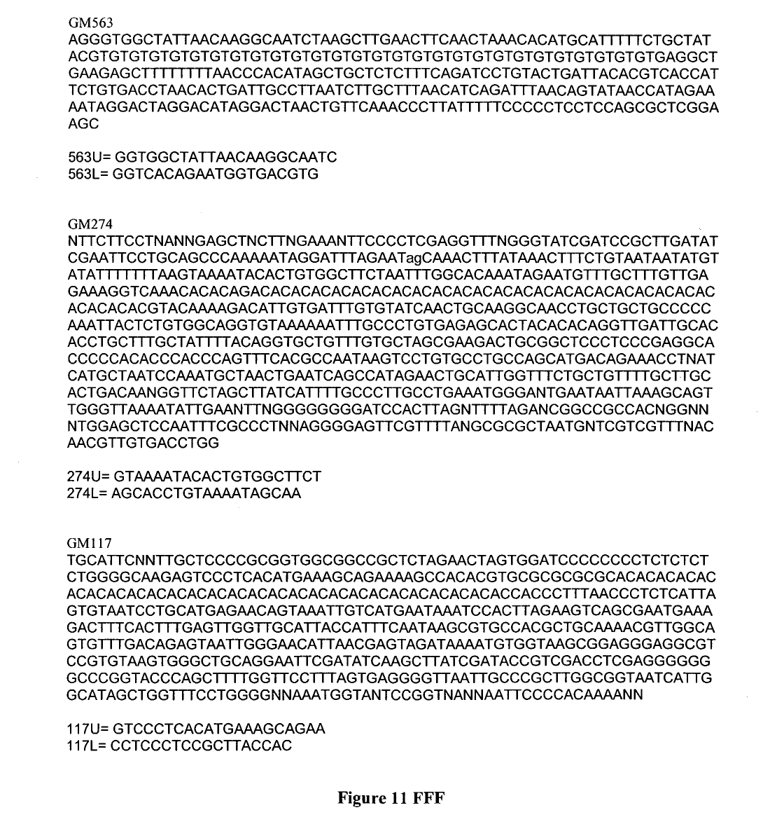

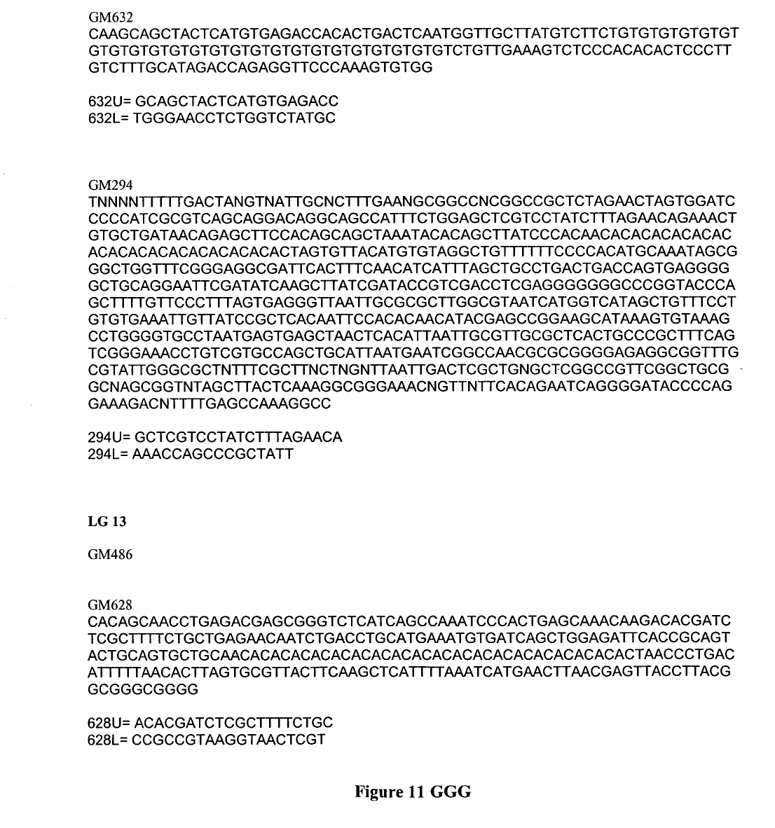

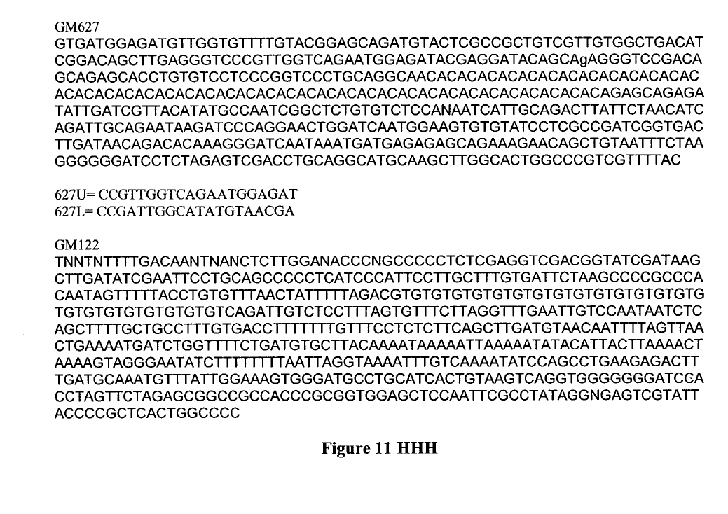

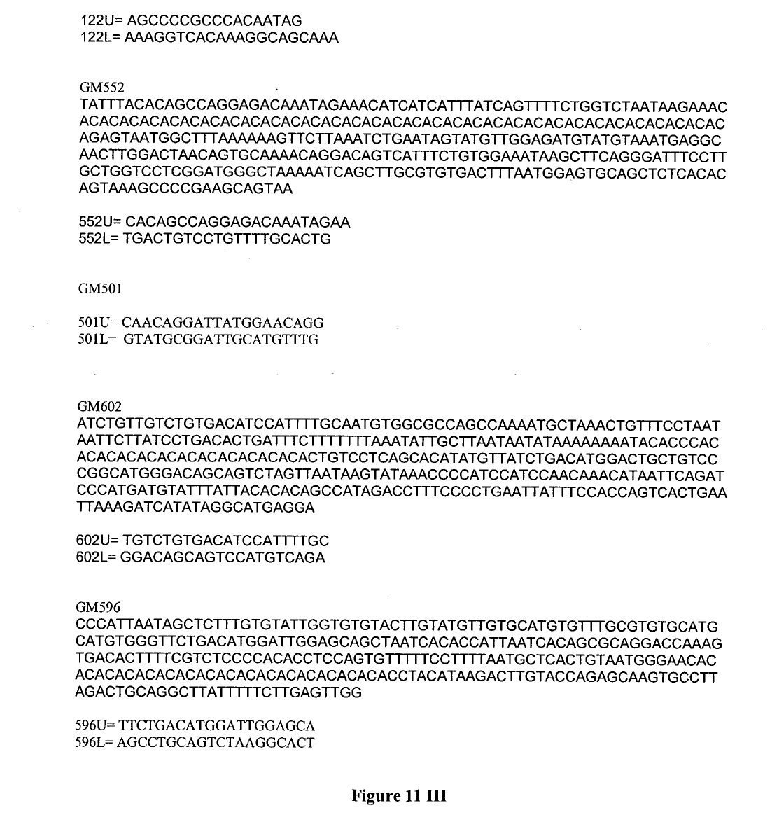

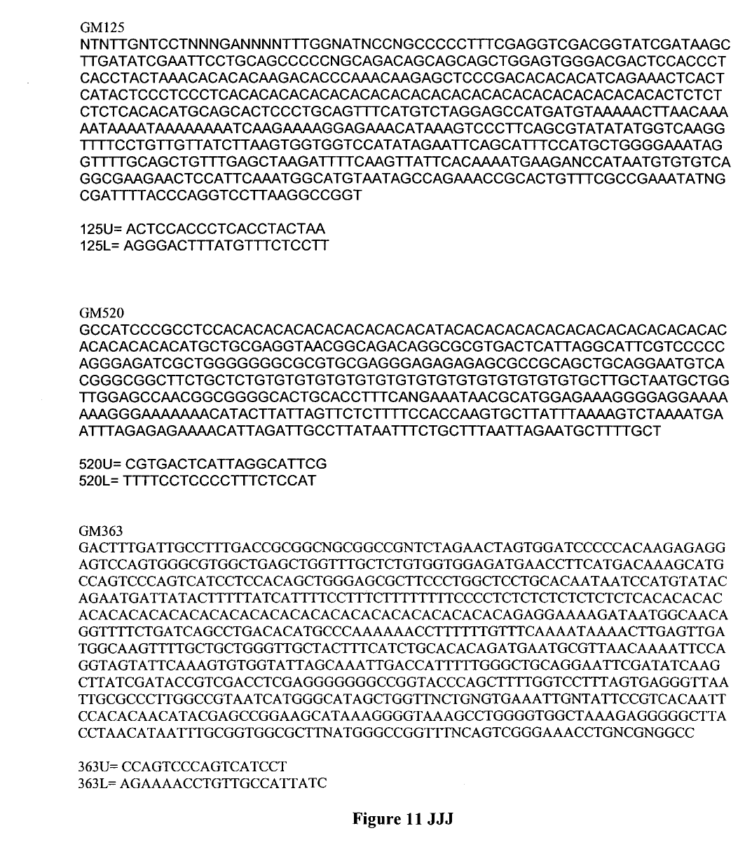

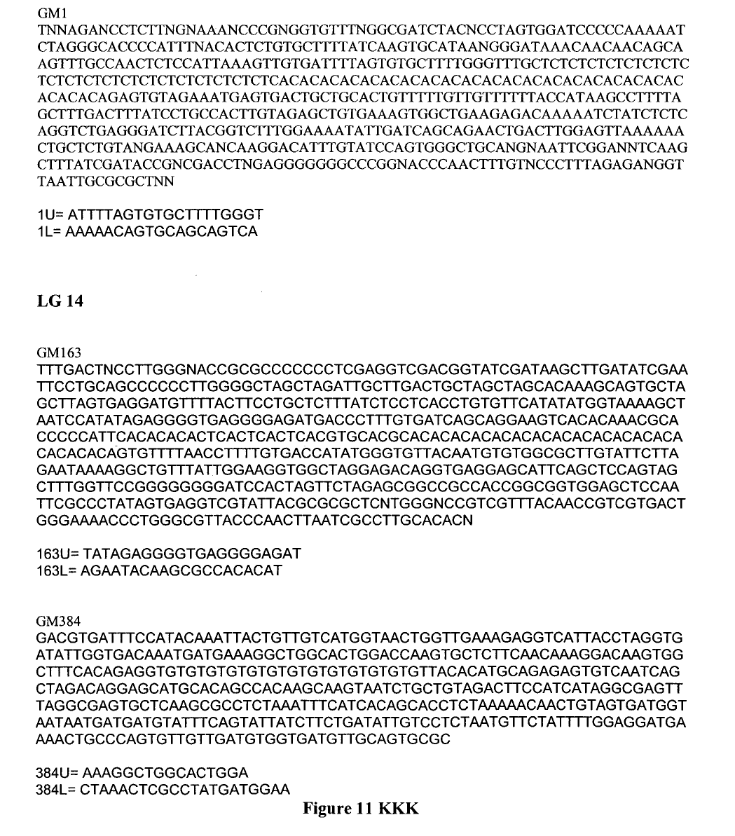

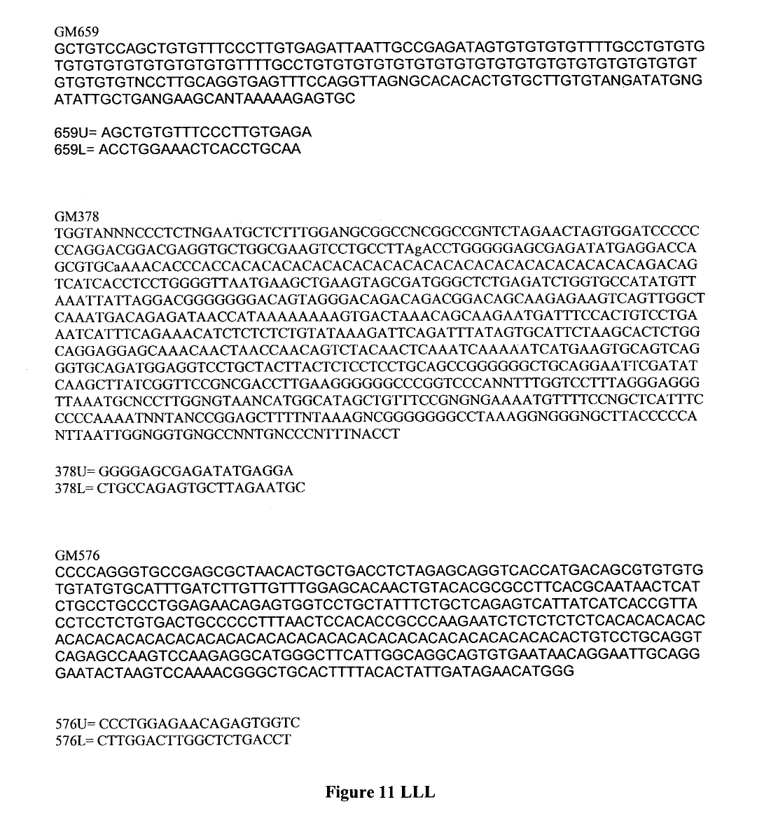

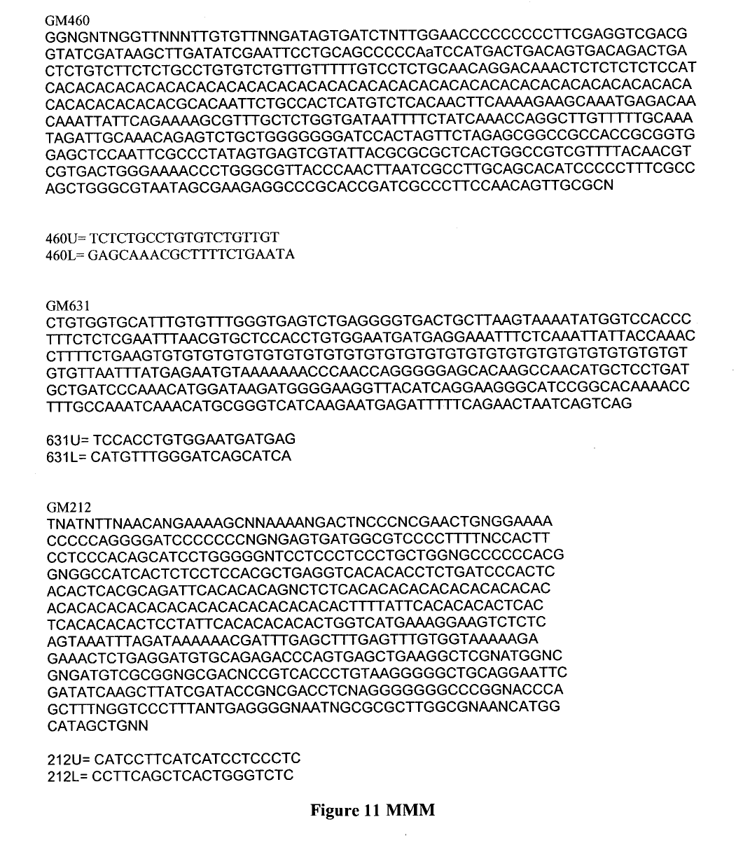

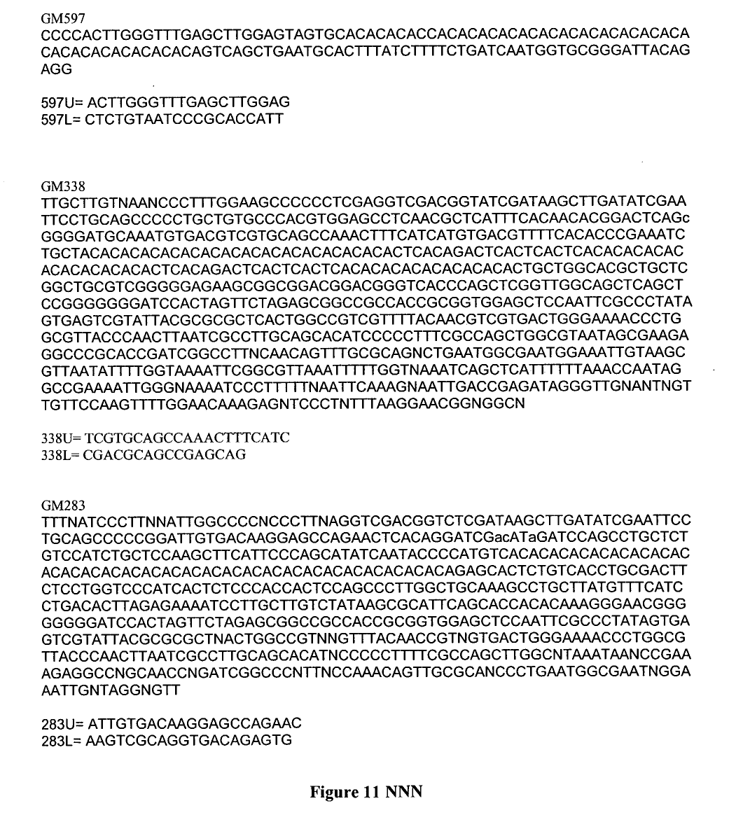

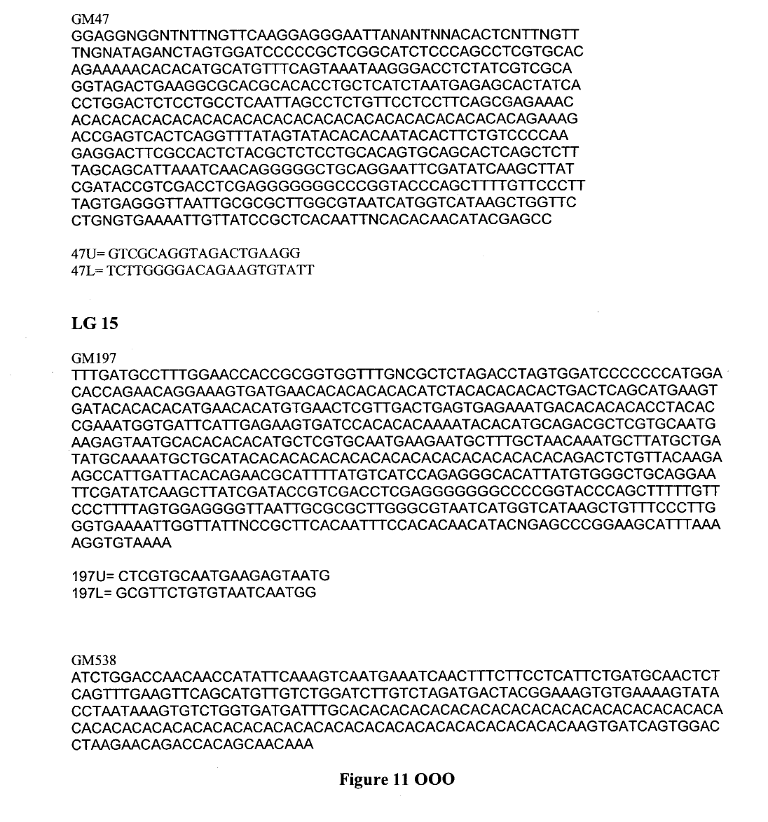

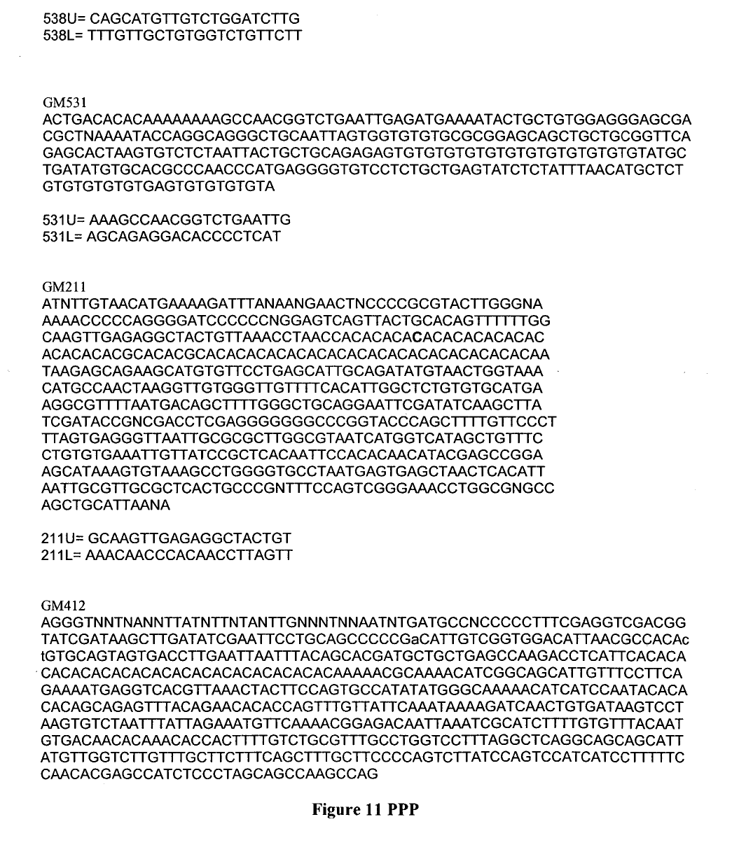

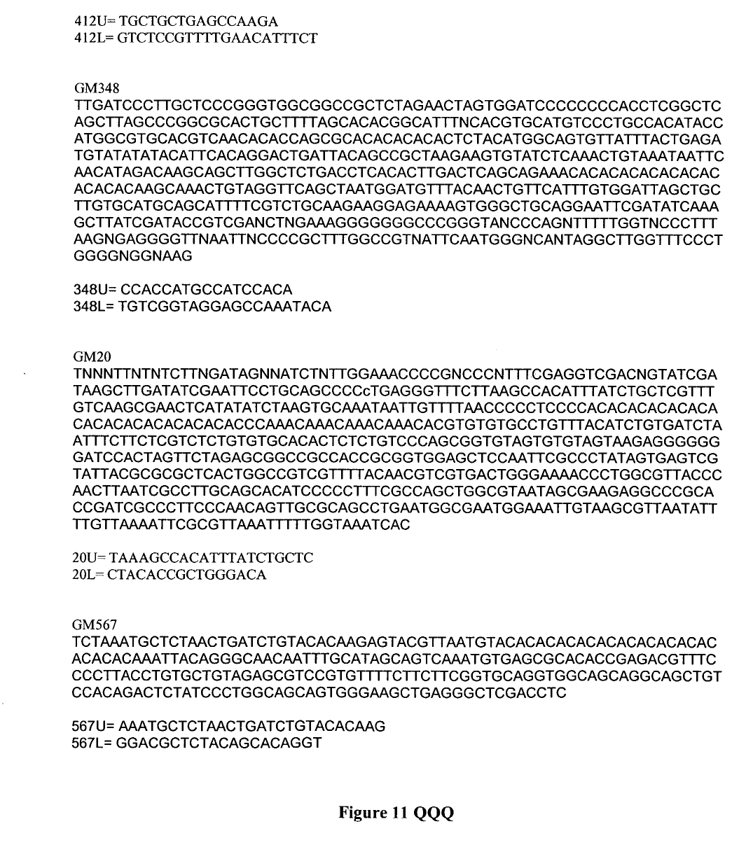

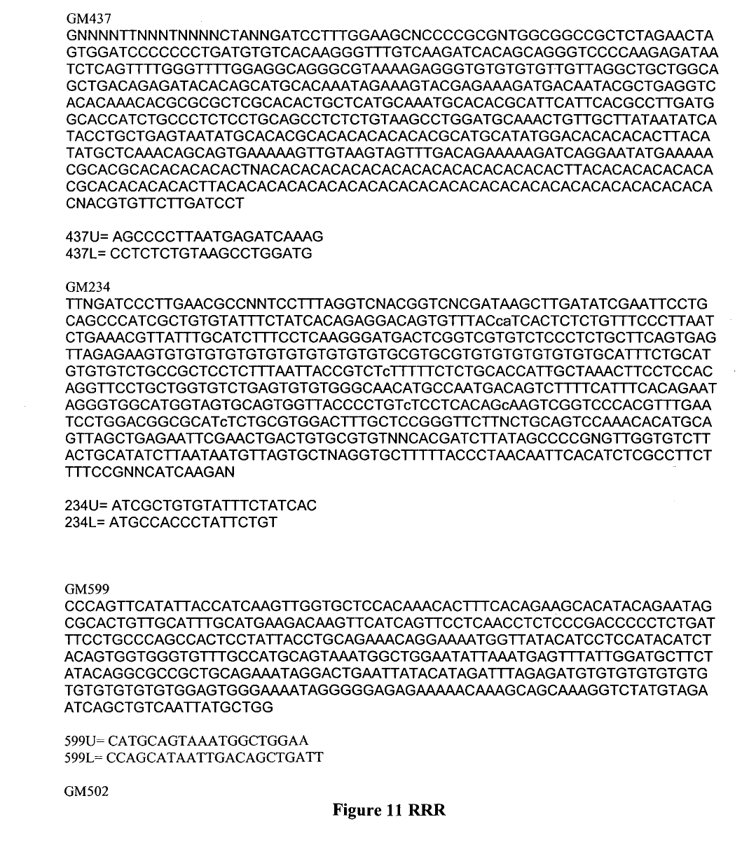

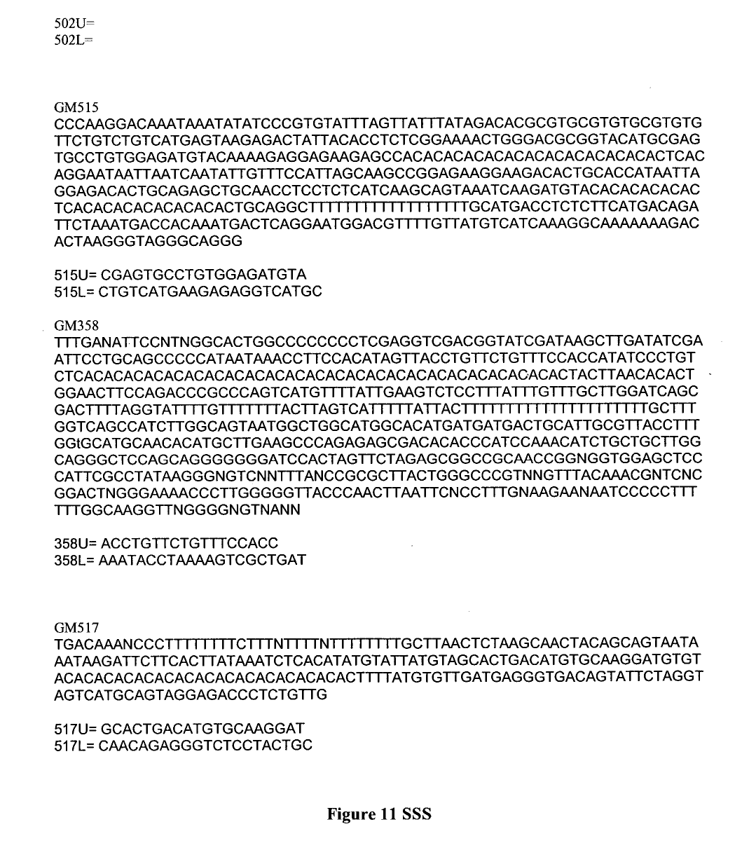

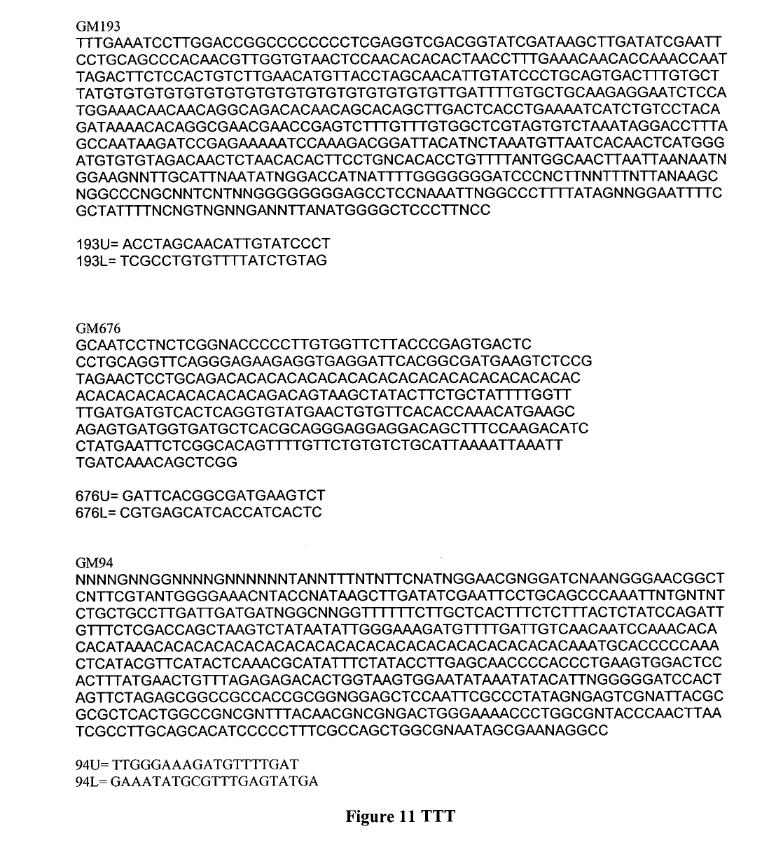

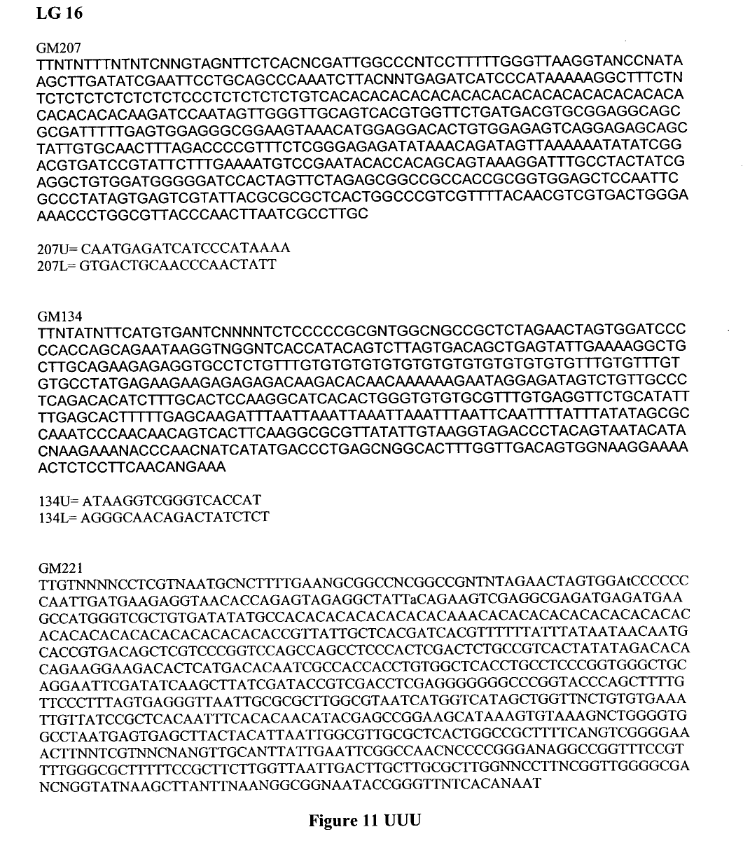

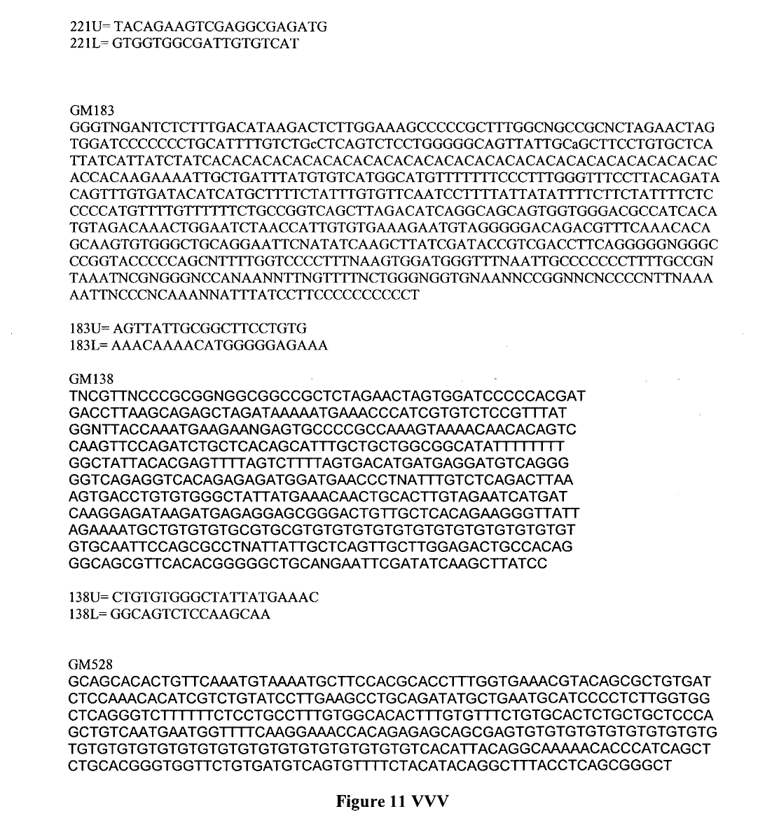

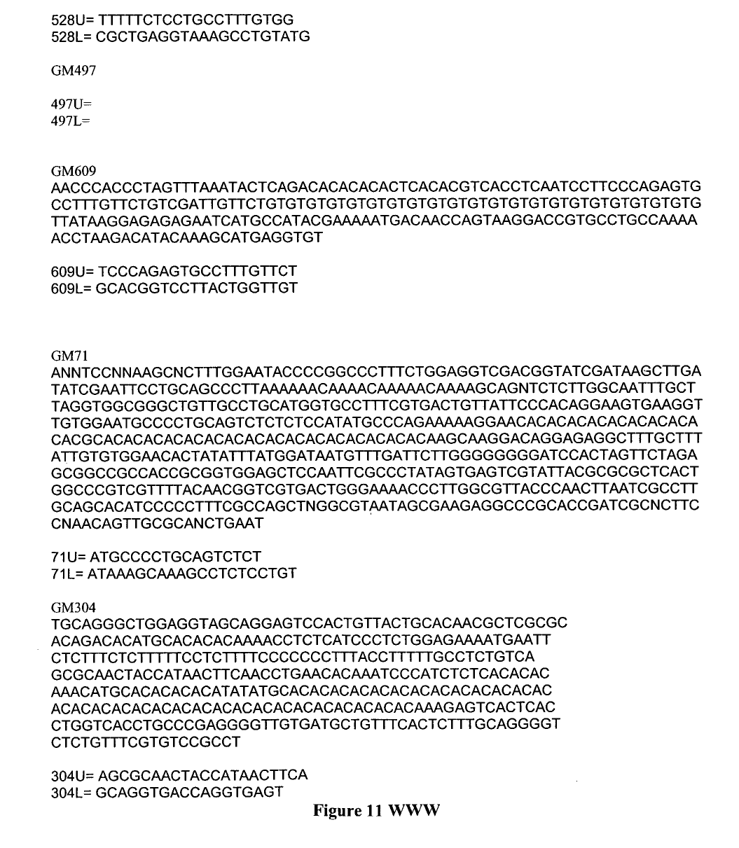

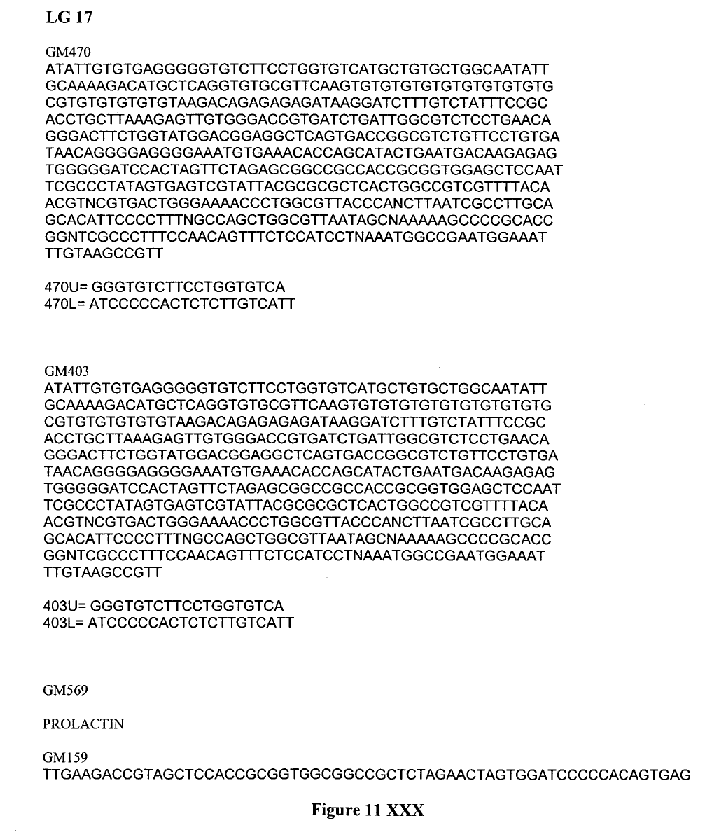

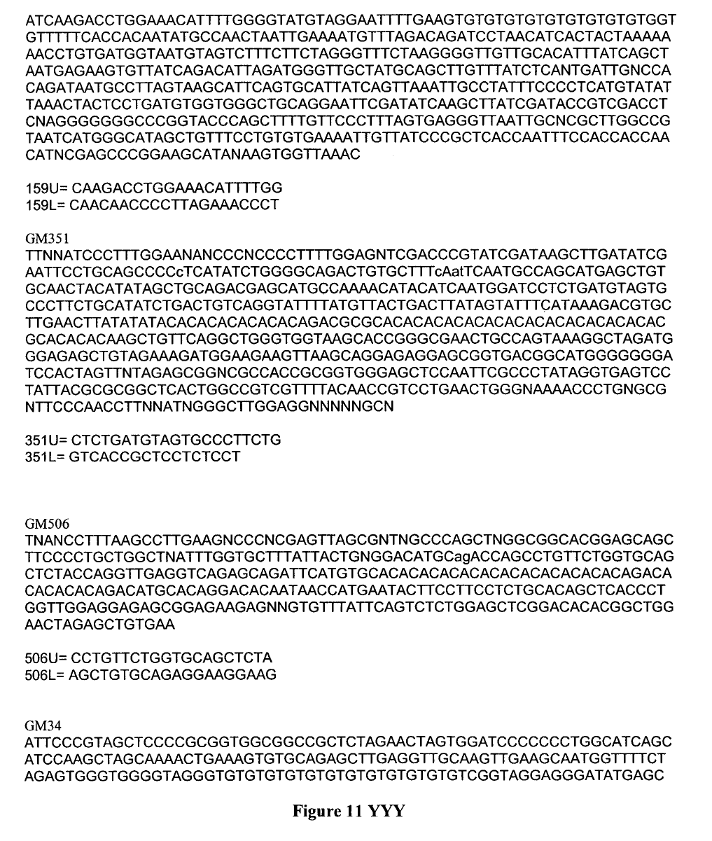

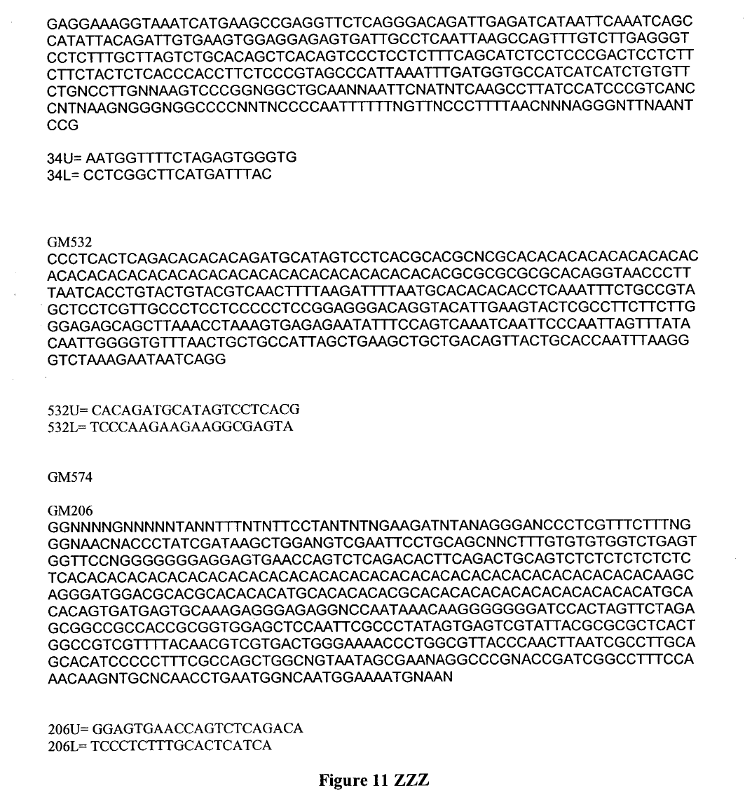

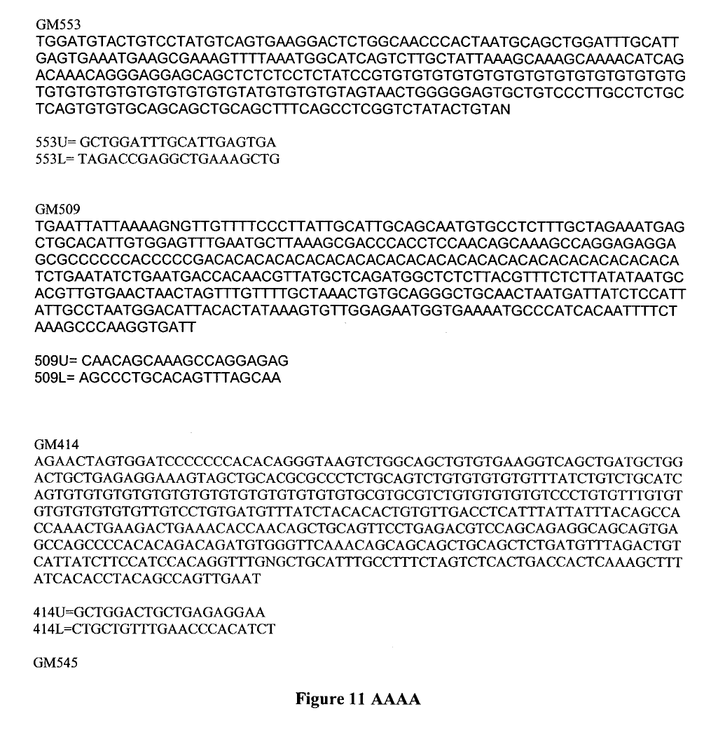

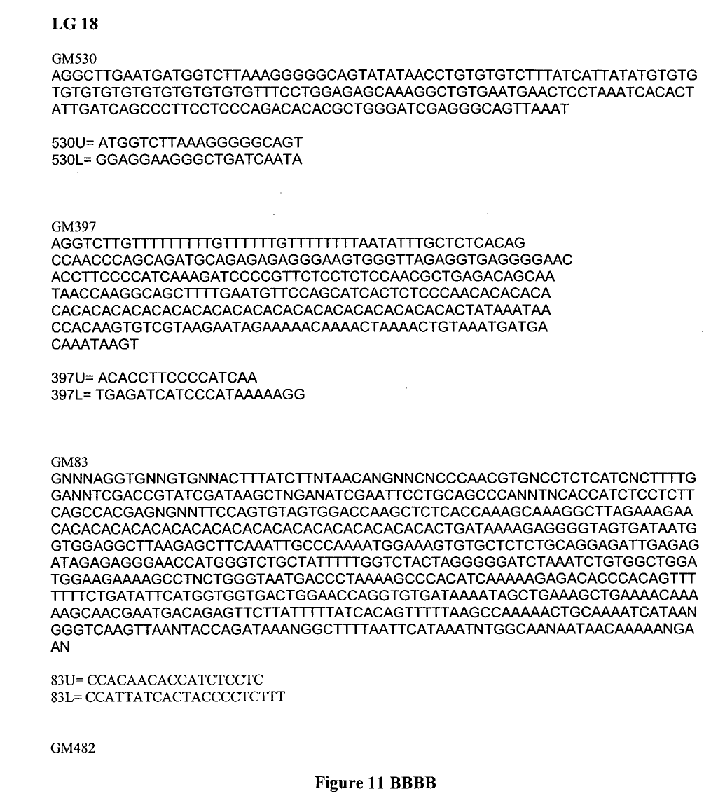

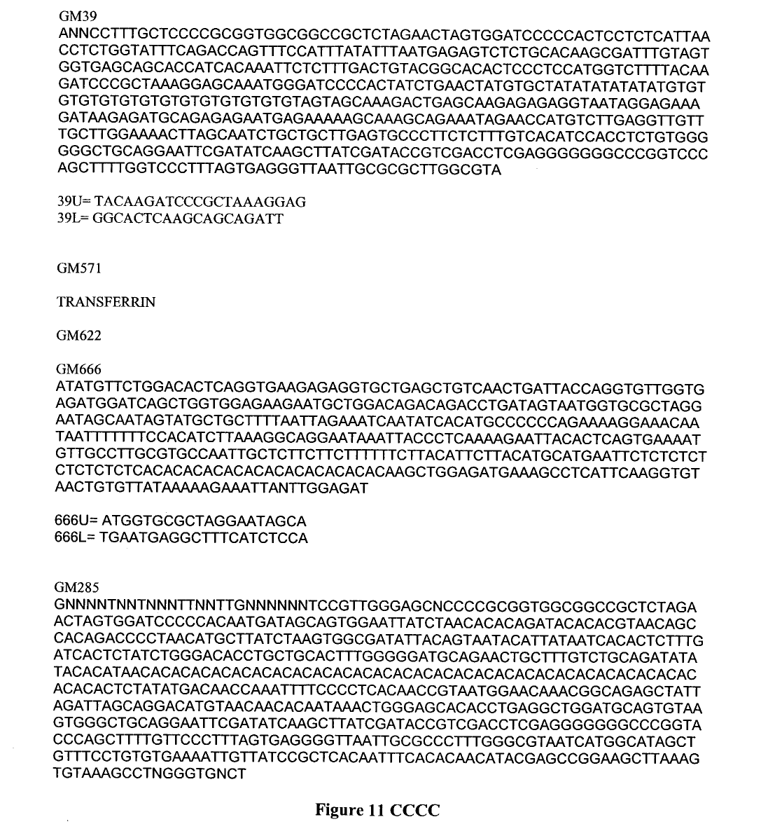

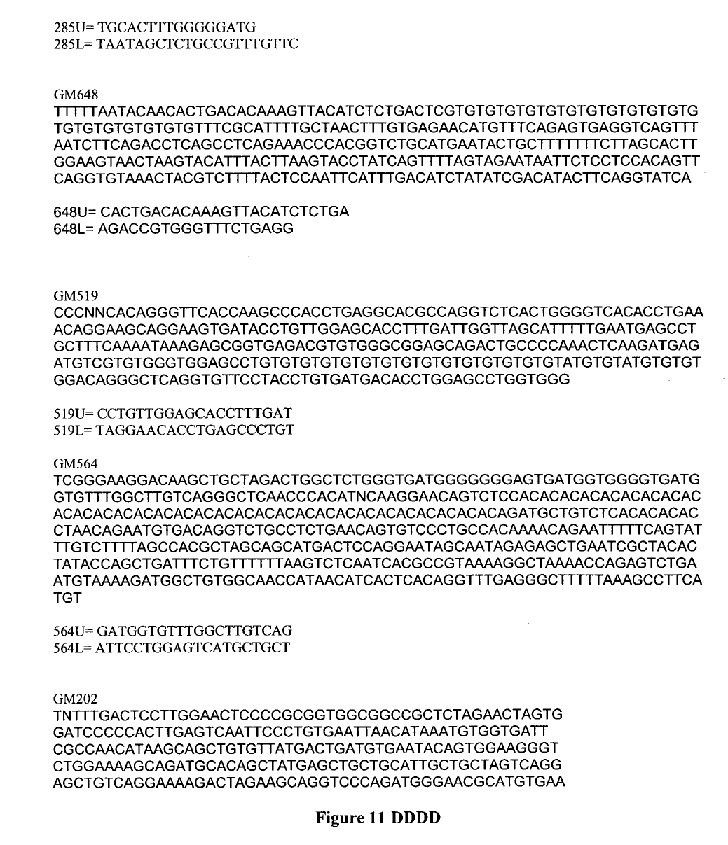

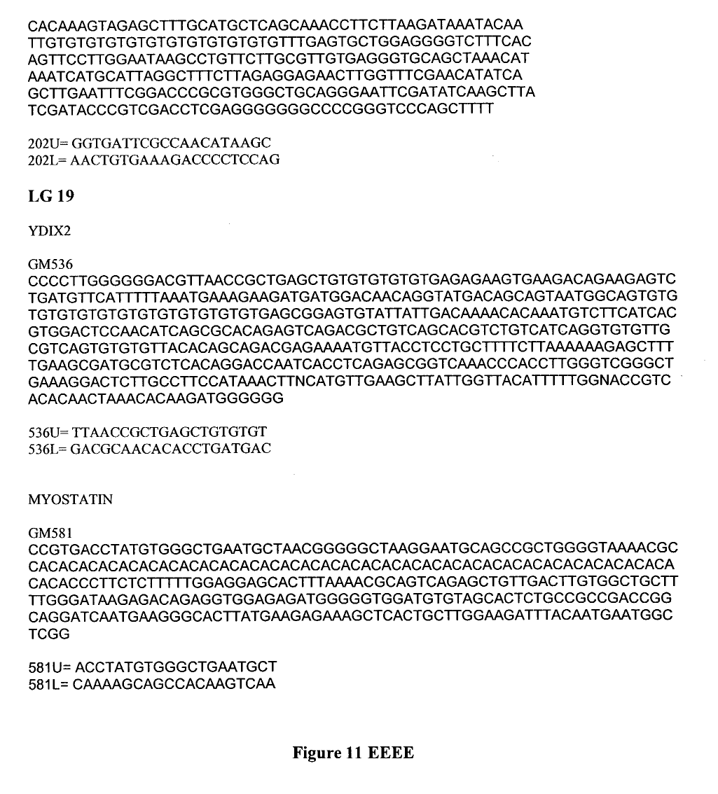

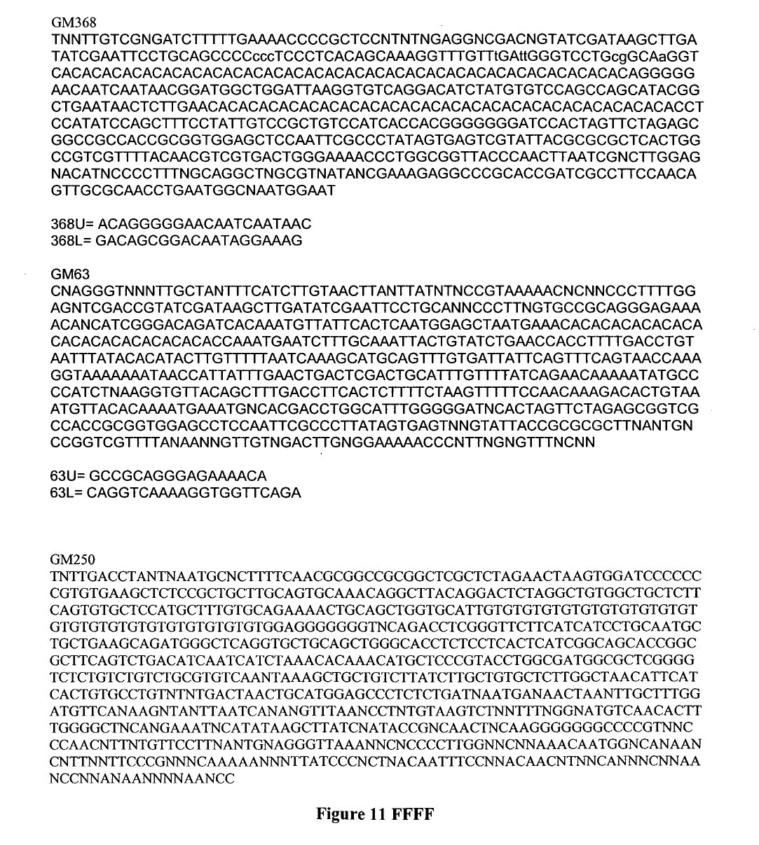

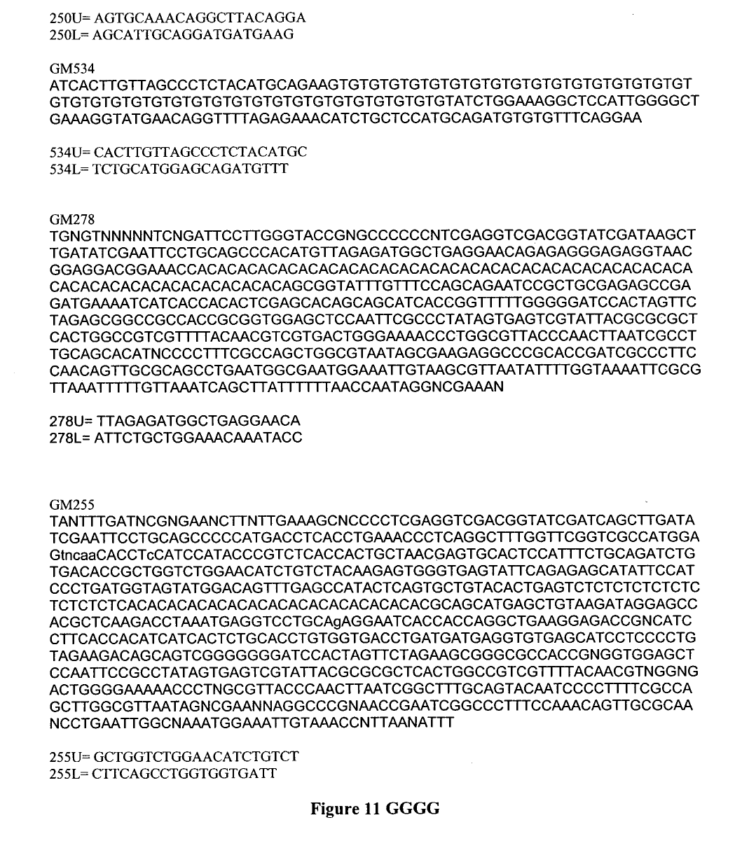

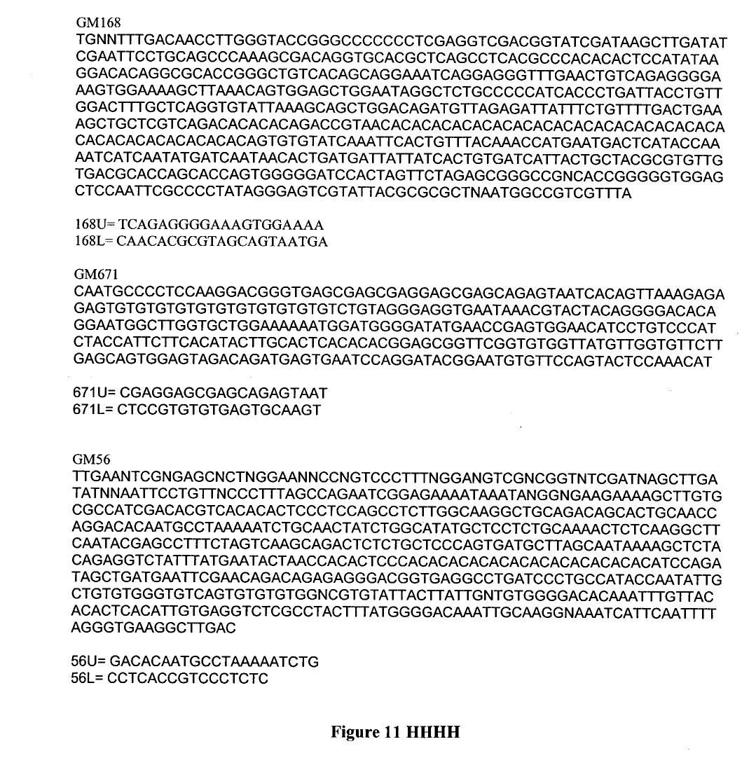

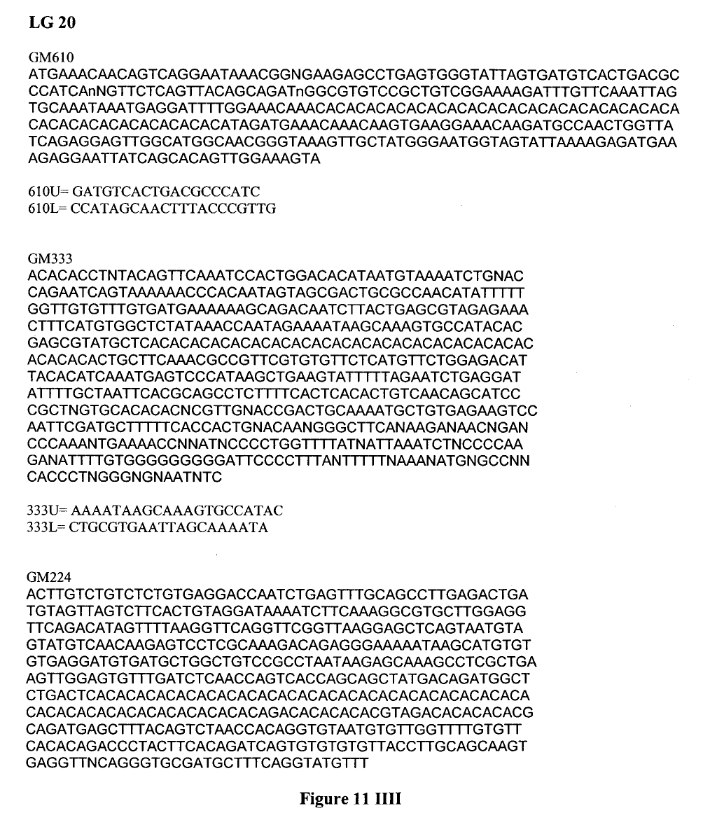

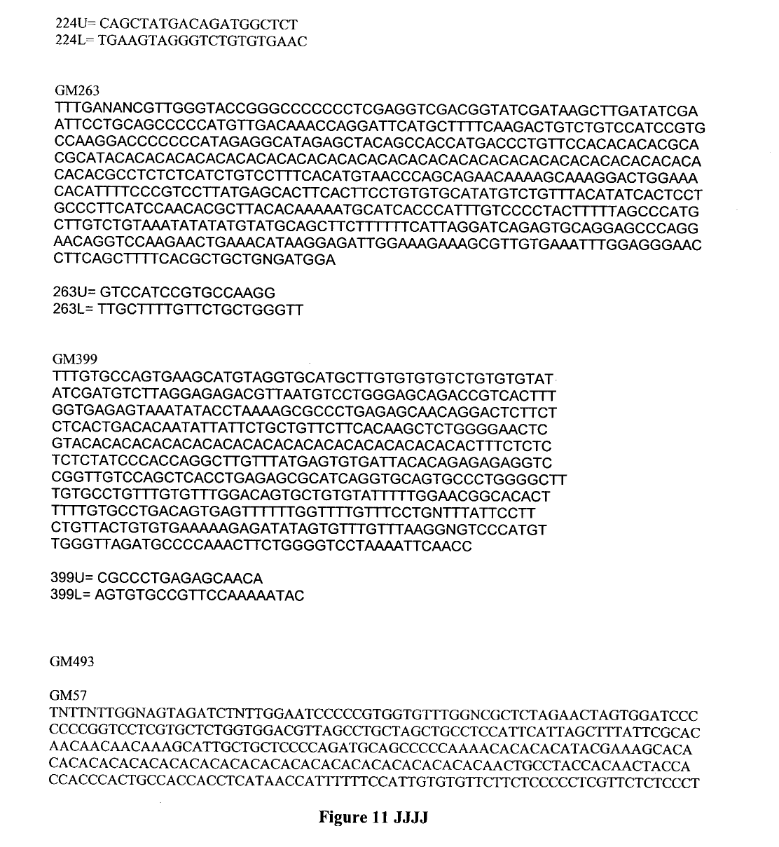

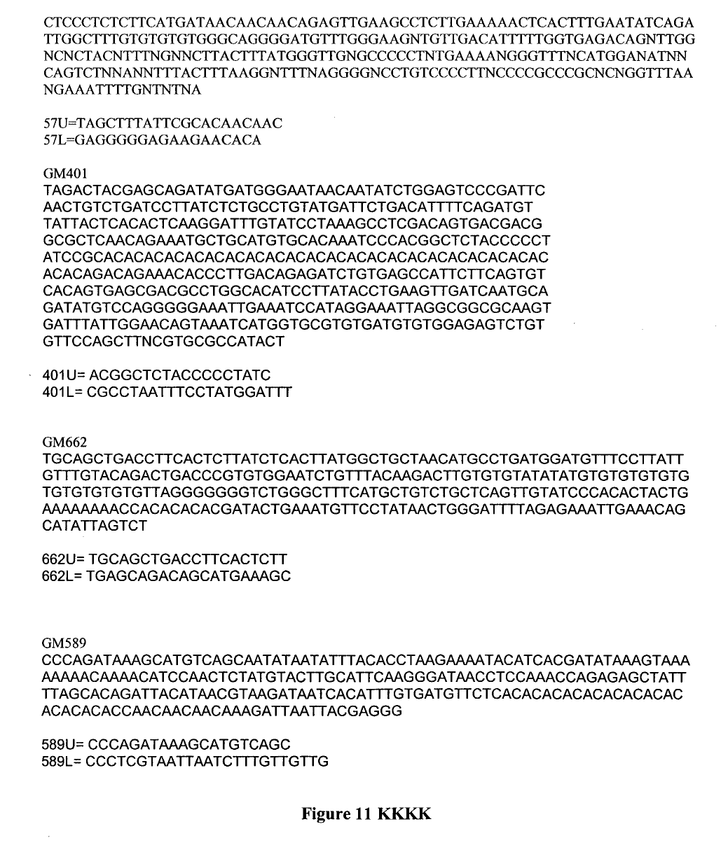

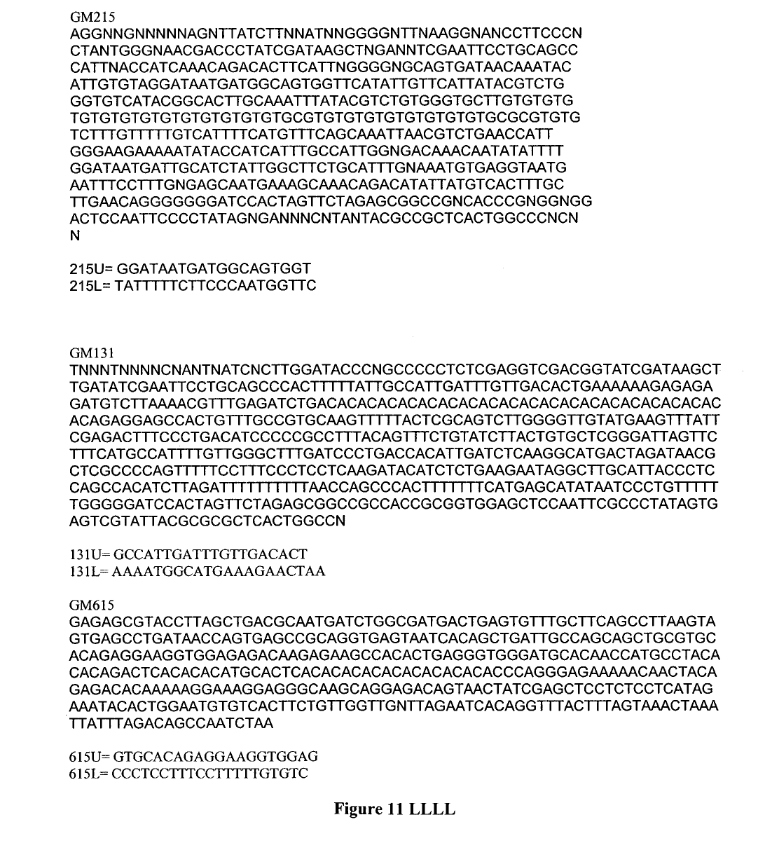

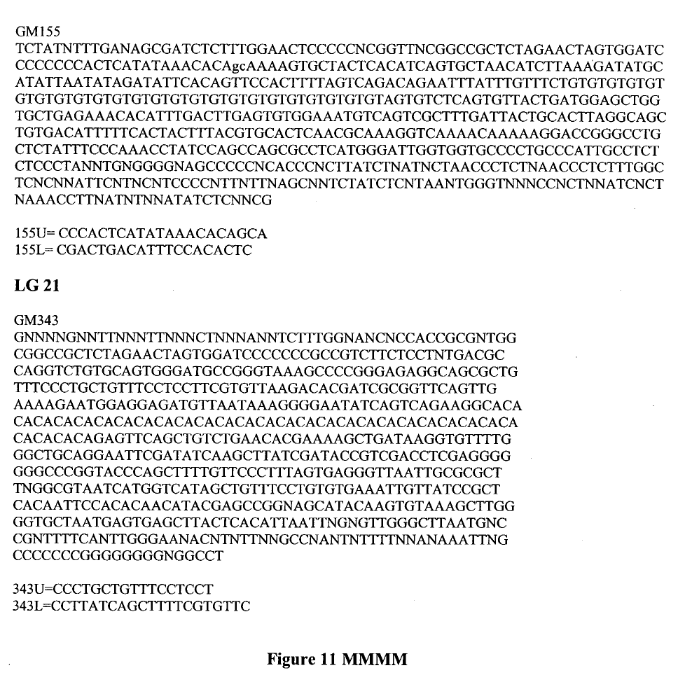

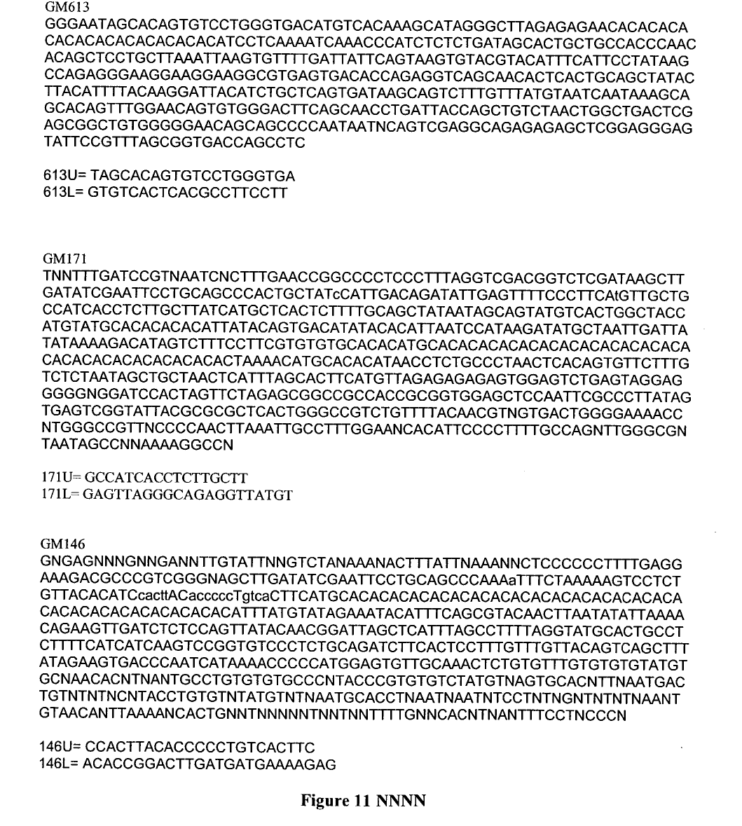

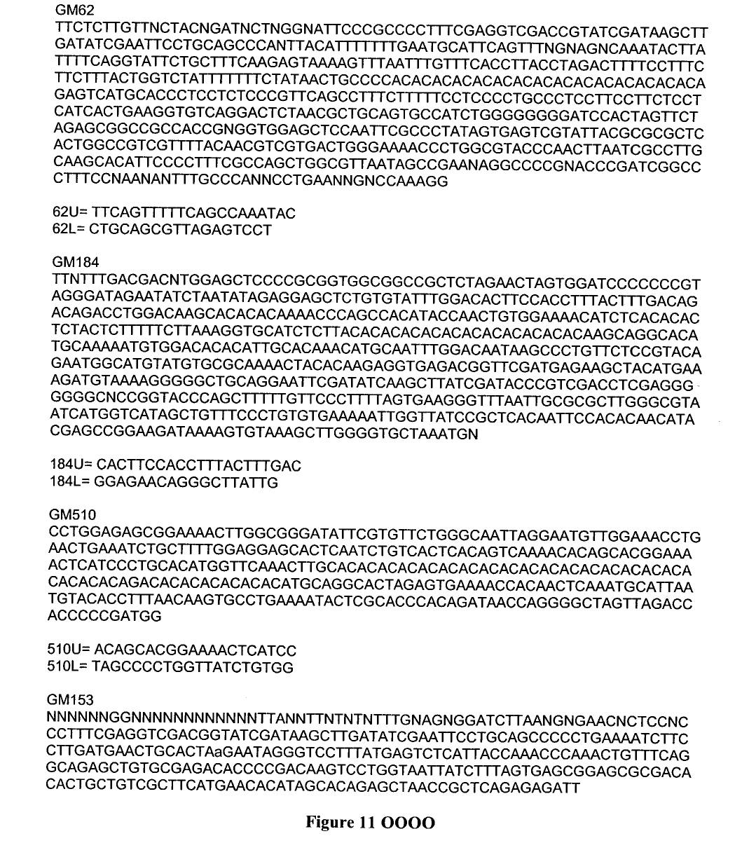

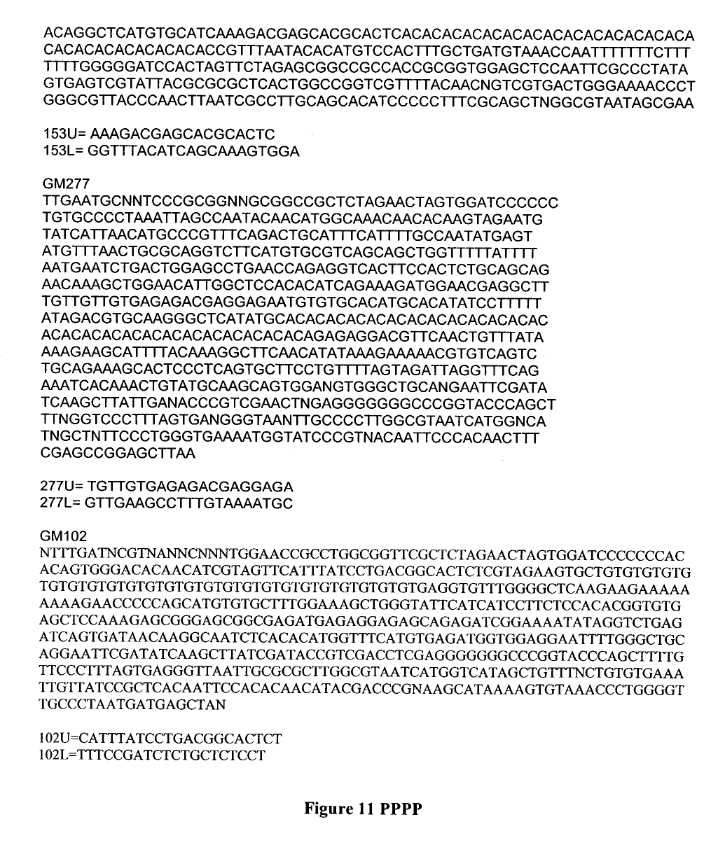

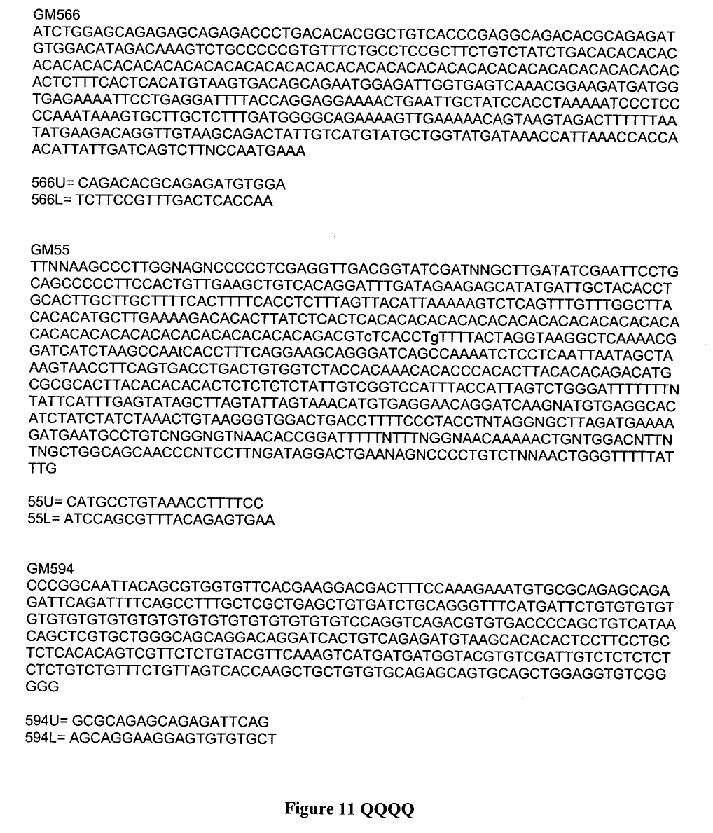

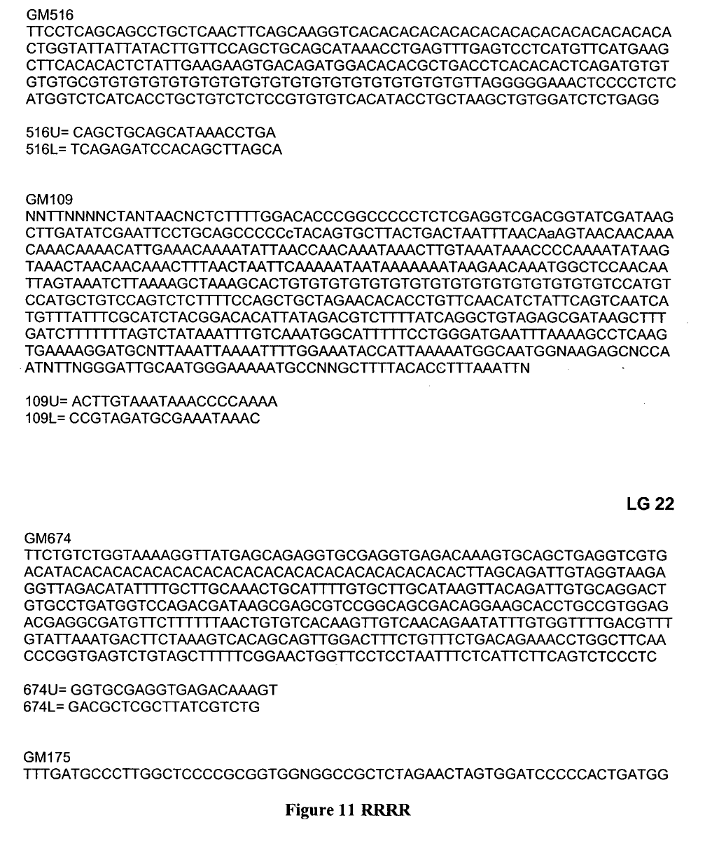

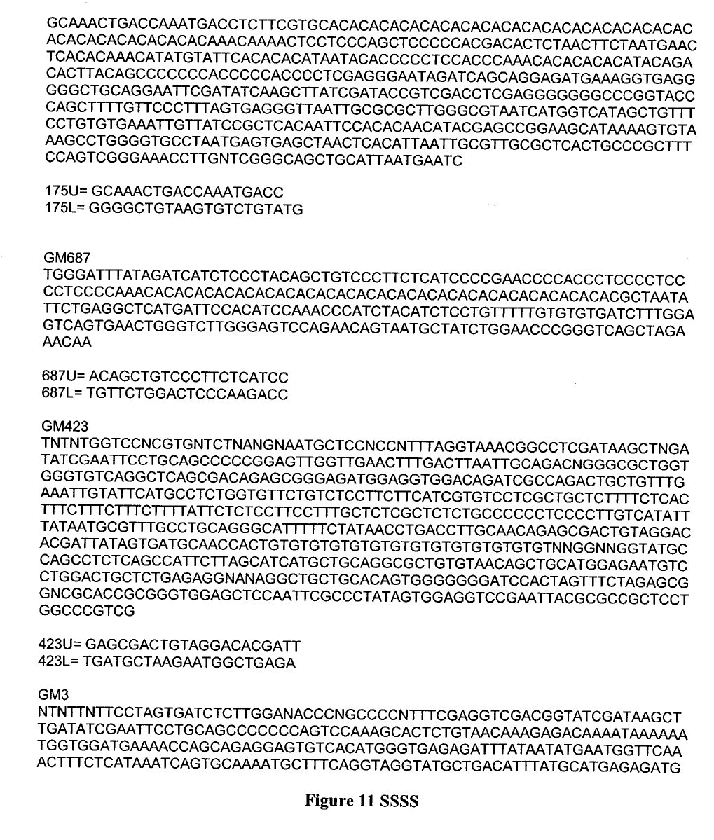

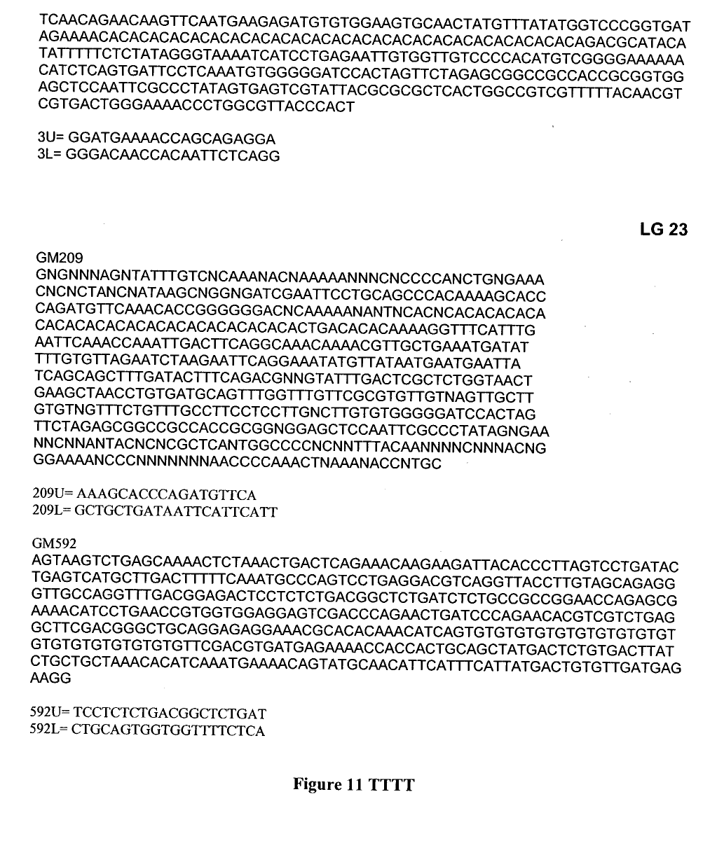

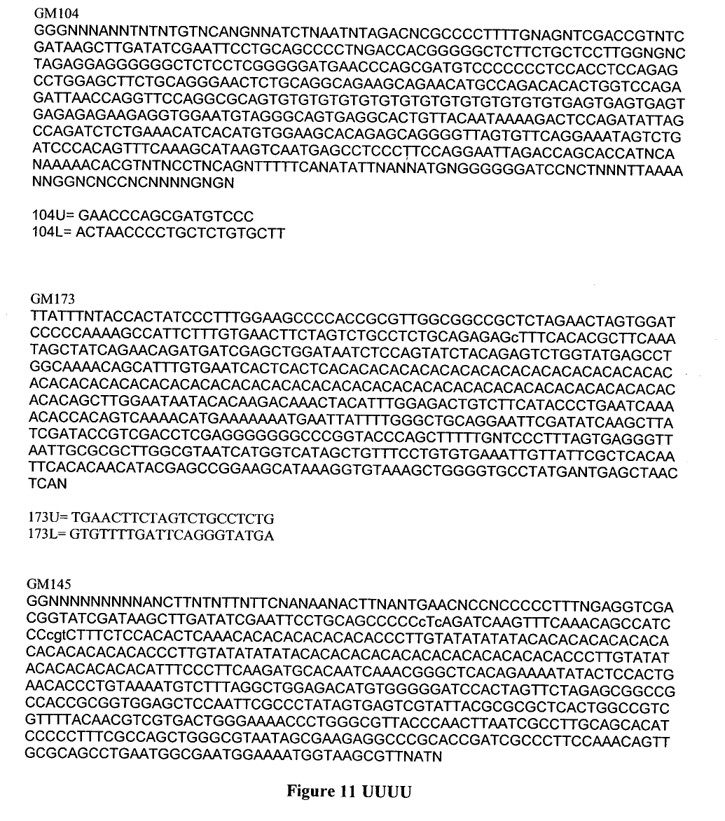

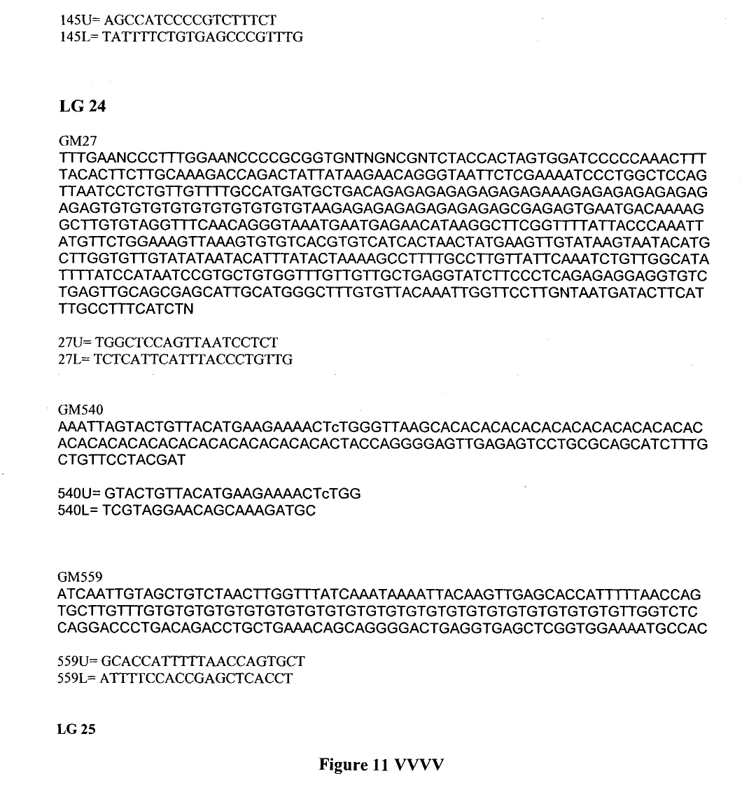

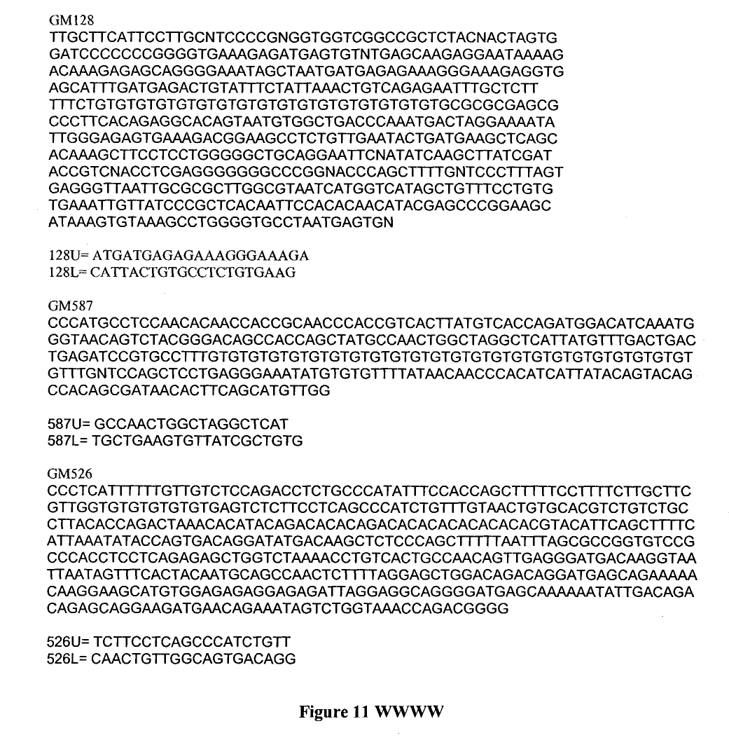

In yet another embodiment, the invention provides an isolated nucleic acid molecule encompassing a microsatellite sequence, where the isolated nucleic acid molecule is selected from the sequences set forth in FIG. 5 (SEQ ID NOS: 309-367) and 11, which correspond to the tilapia. Also provided are nucleic acid molecules that hybridize to a microsatellite nucleic acid molecule set forth in FIGS. 5 and 11, or its complement under highly stringent hybridization conditions. FIG. 11 shows isolated nucleic acid molecule encompassing tilapia microsatellite nucleotide sequences and corresponding primers consecutively designated SEQ ID NOS: 473-1377.

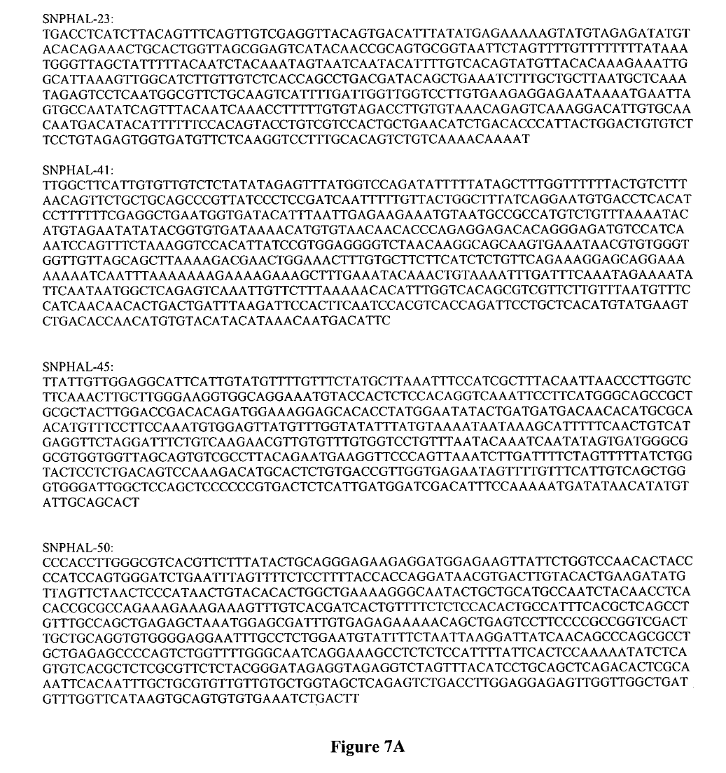

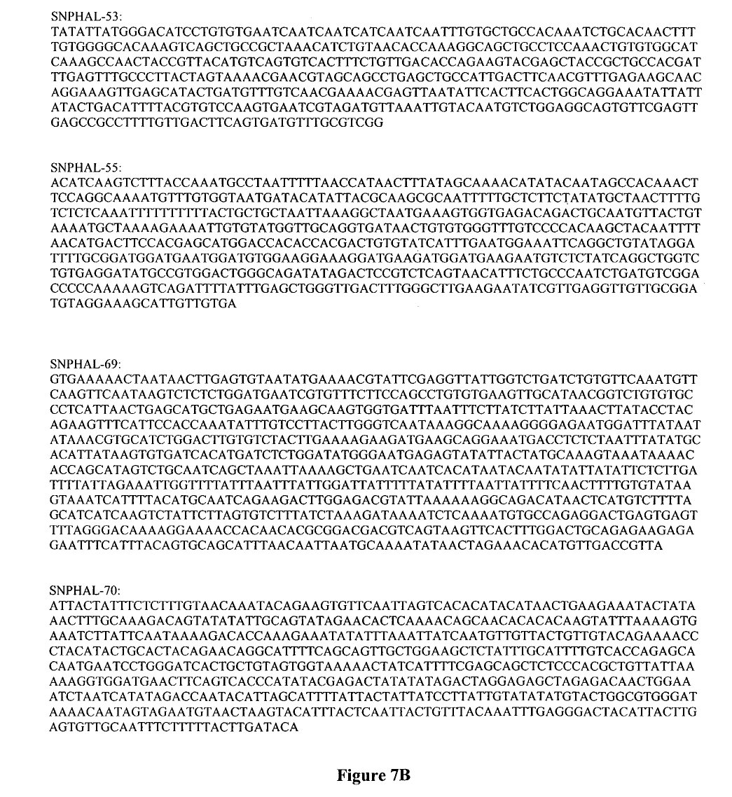

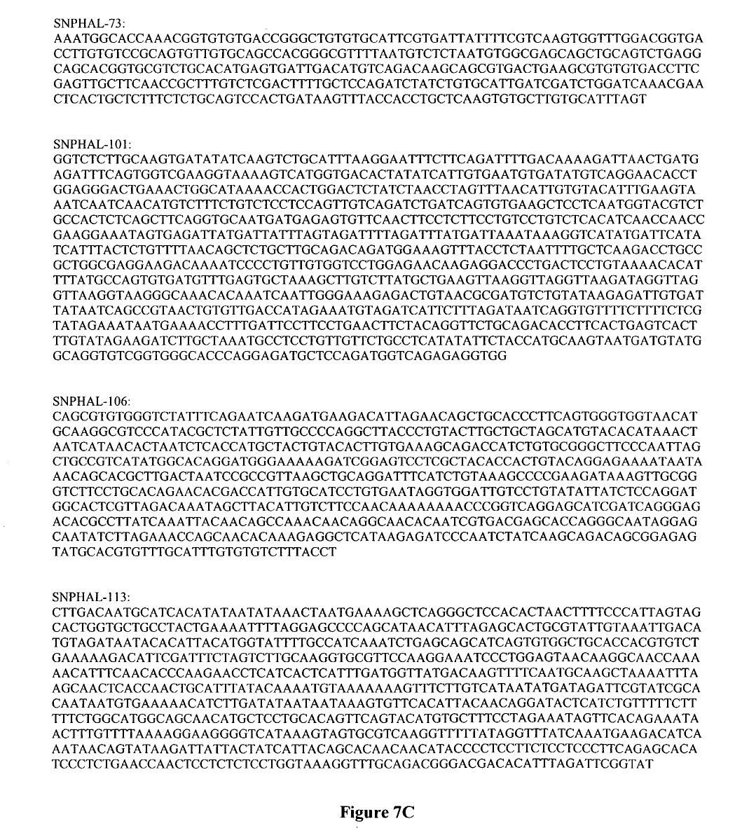

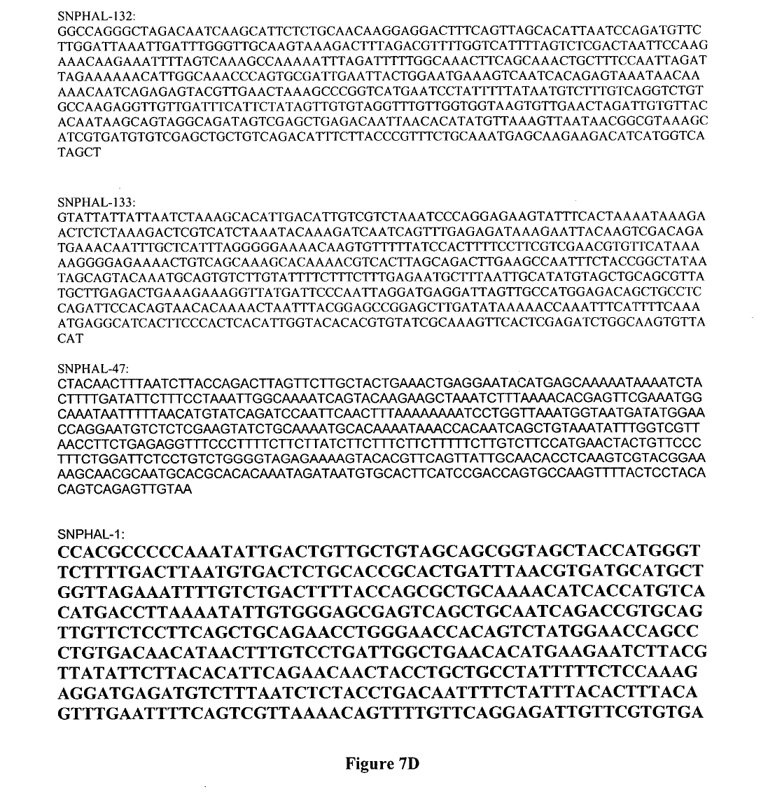

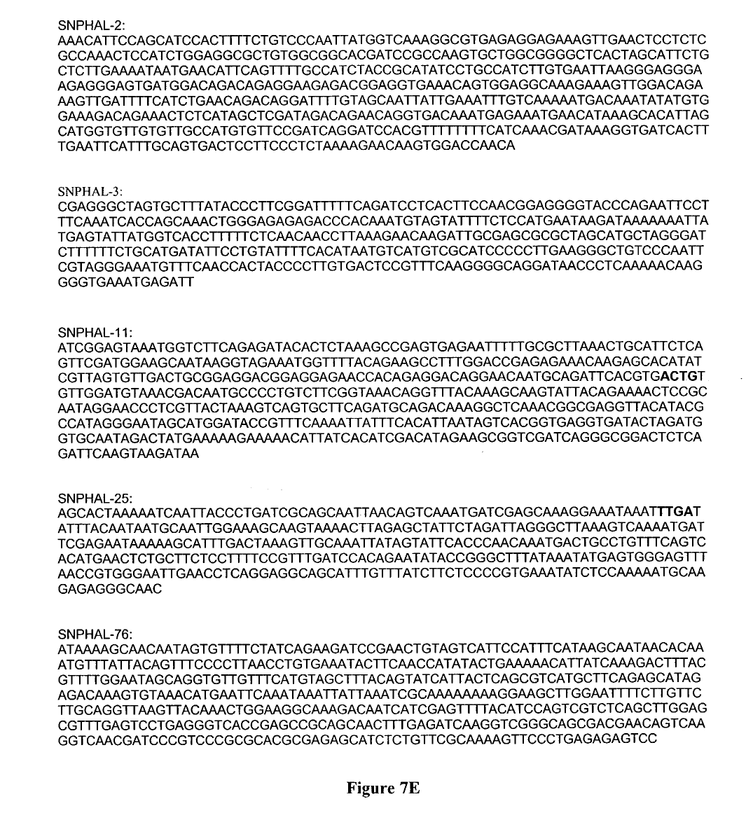

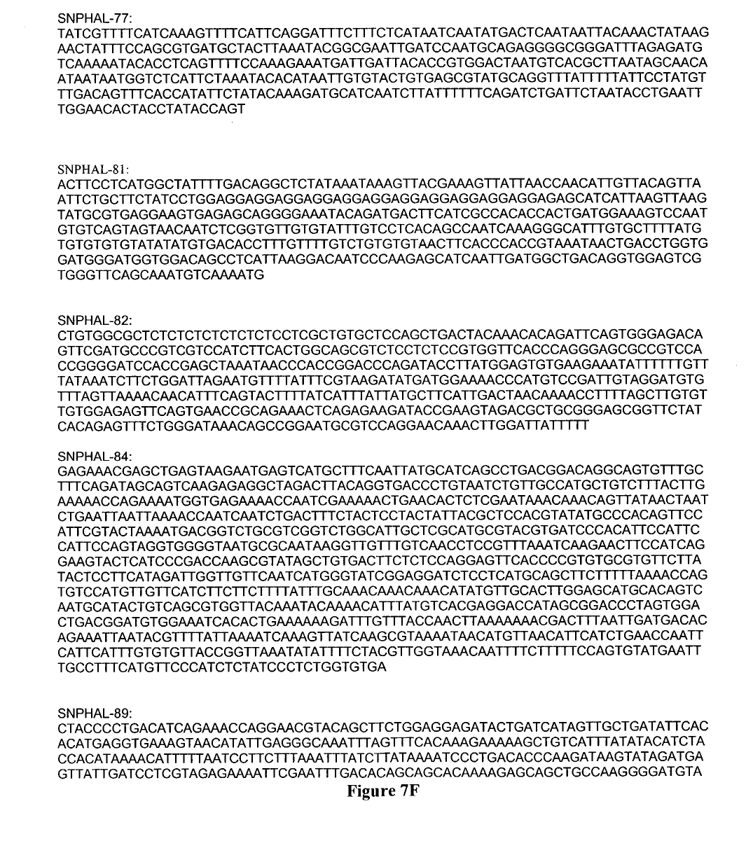

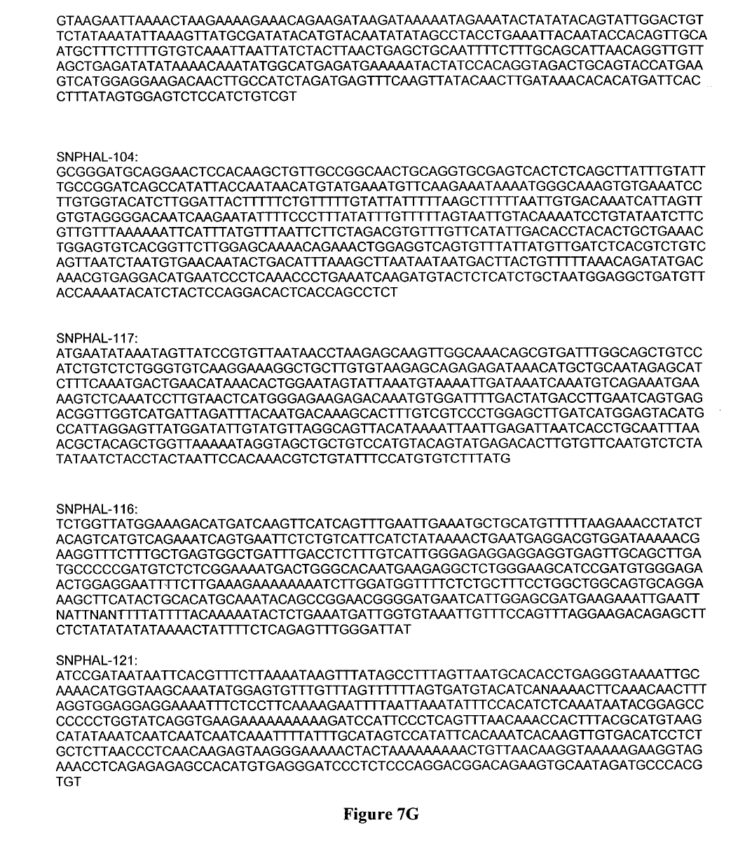

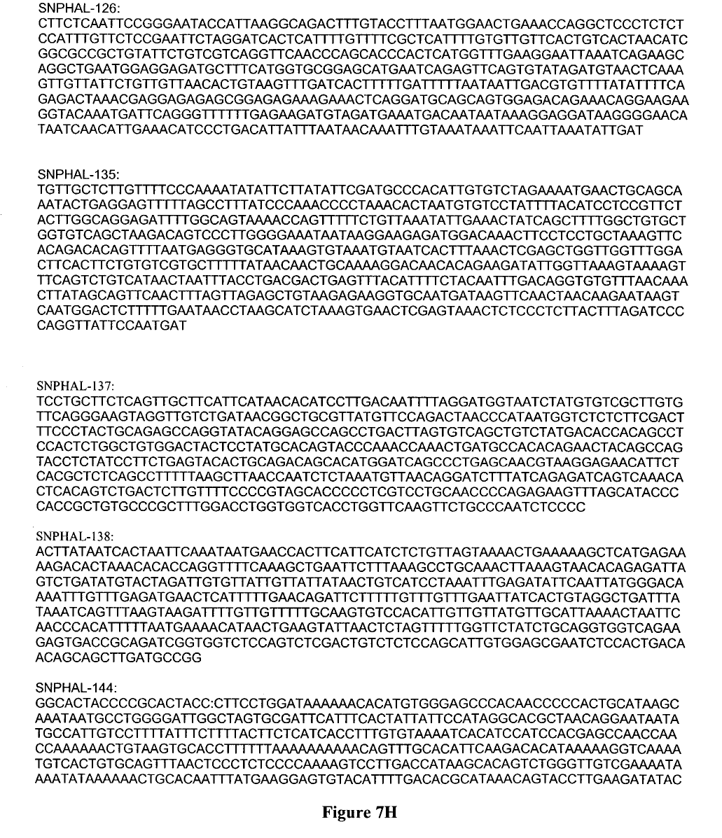

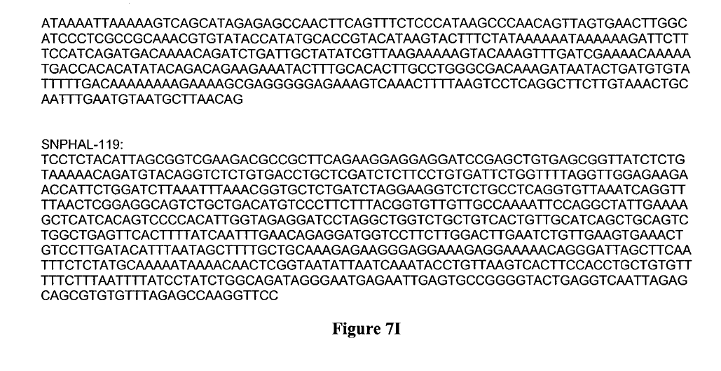

In yet another embodiment, the invention provides an isolated nucleic acid molecule encompassing encompassing a single nucleotide polymorphism (SNP), where the isolated nucleic acid molecule has a nucleotide sequence selected from the group designated SEQ ID NOS: 374-409 and set forth in FIG. 7, which correspond to halibut.

As used herein, the term “halibut” refers to organisms that belong the order Pleuronectifores, family Pleuronectidae, and genus Hippoglossus and species Hippoglossus hippoglossus, a large saltwater flatfish that can be up to 4 meters in length and is found in the North Atlantic and North Eastern Pacific.

Also provided are nucleic acid molecules that hybridize to the nucleic acid molecule selected from the group designated SEQ ID NOS: 374-409 or its complement under highly stringent hybridization conditions.

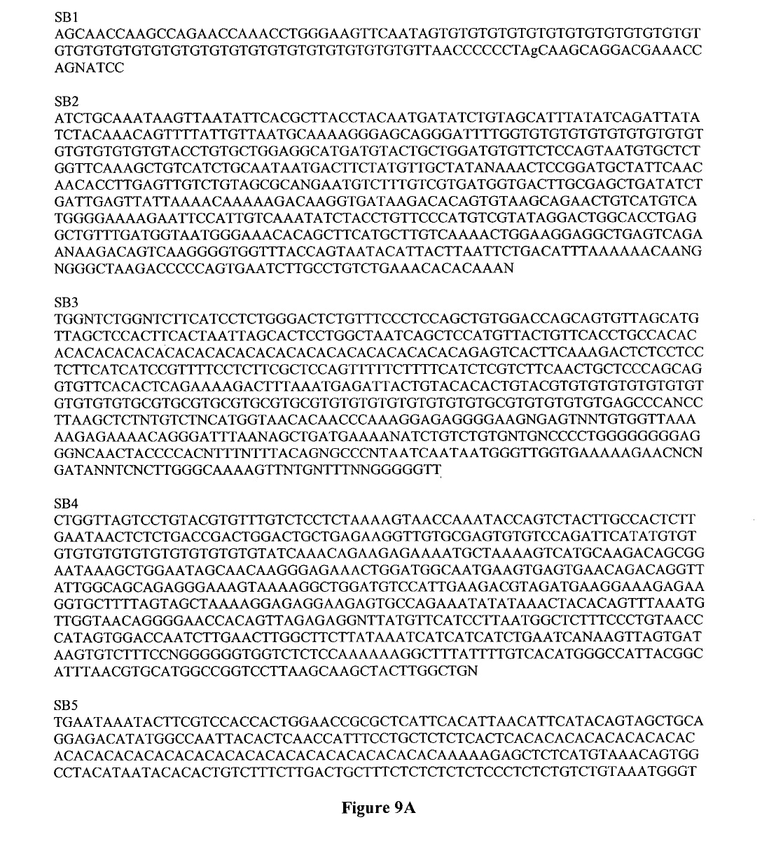

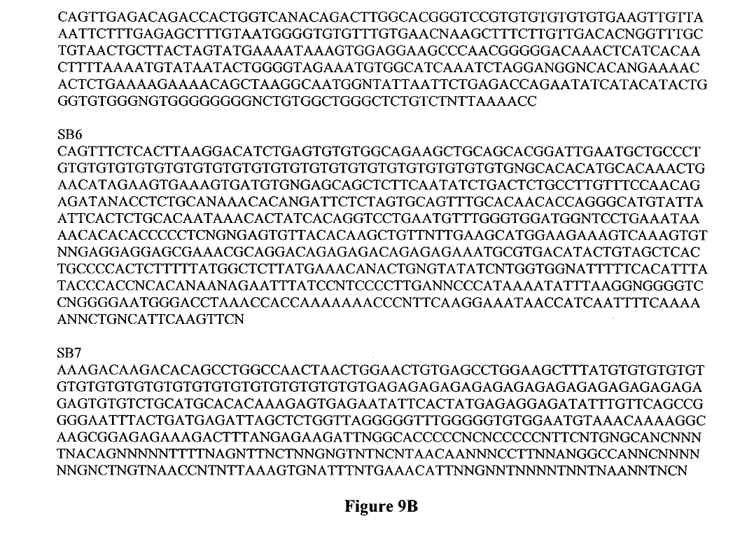

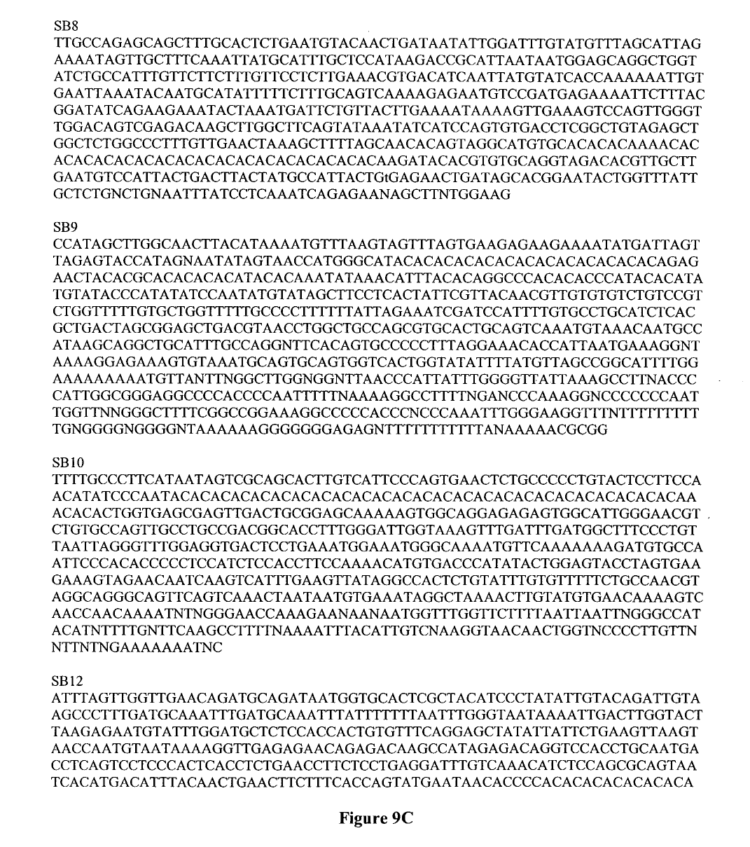

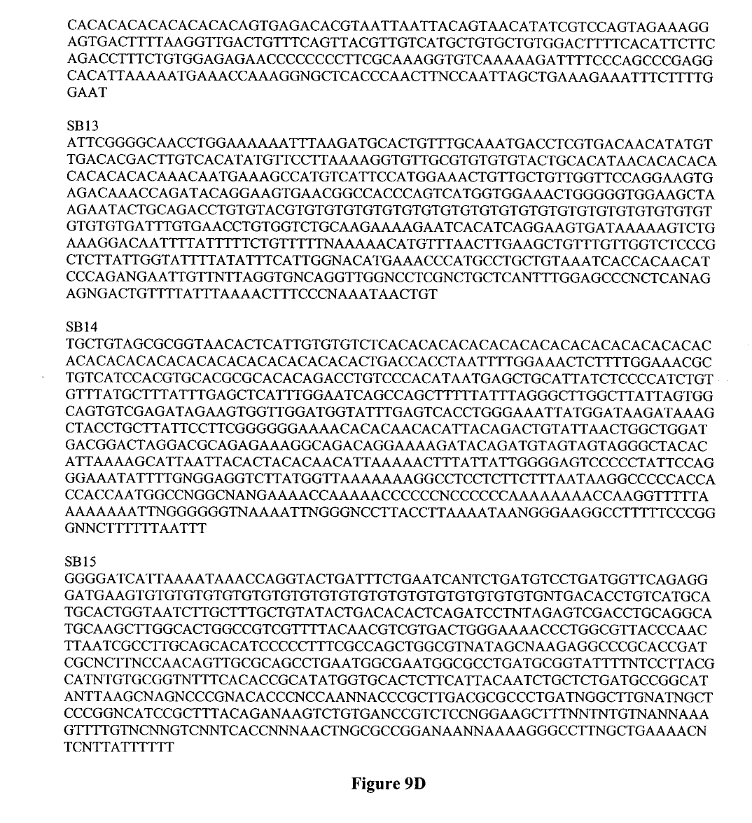

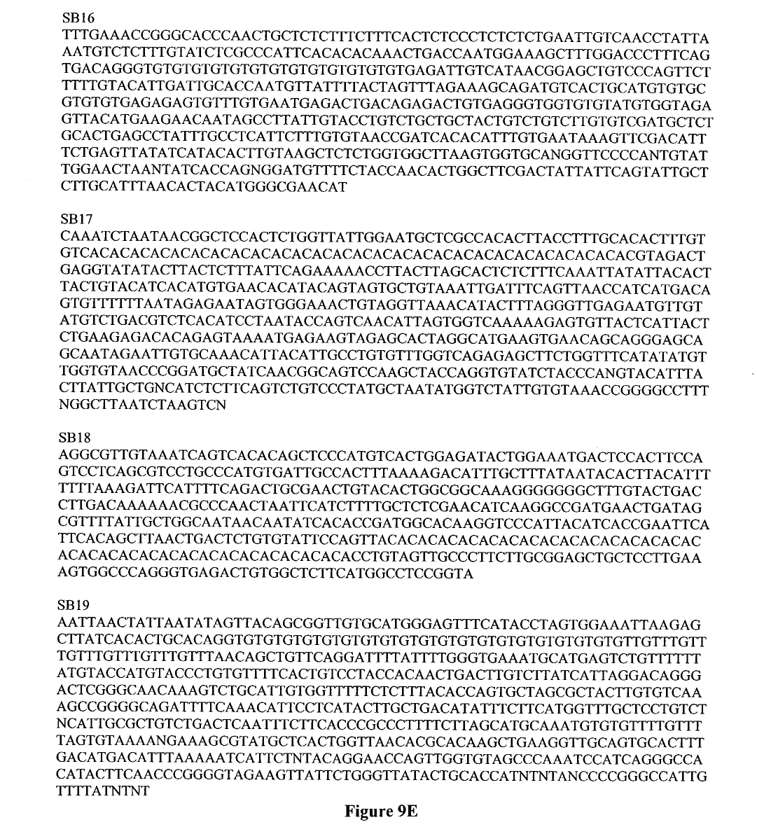

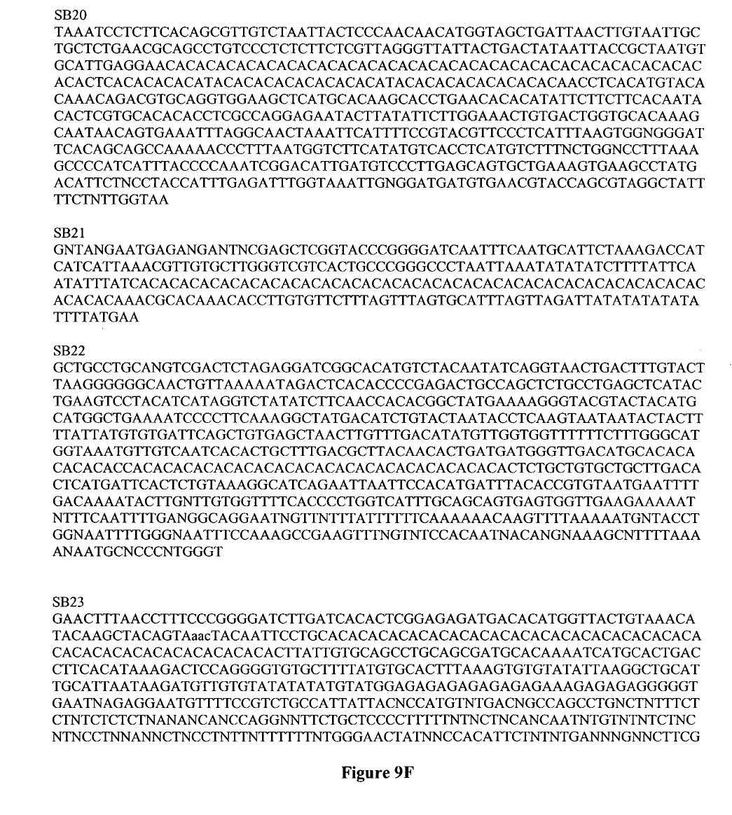

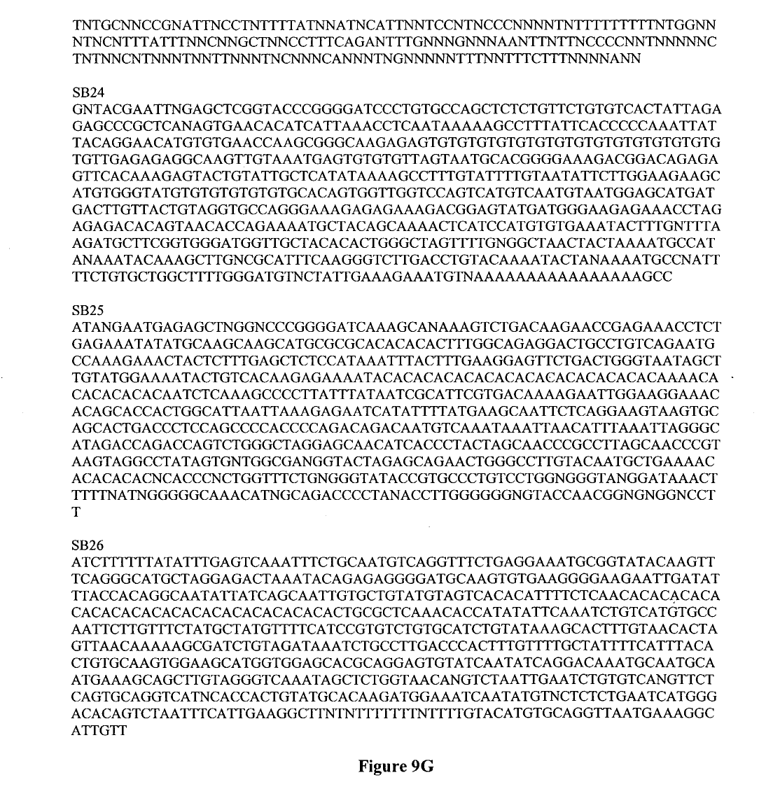

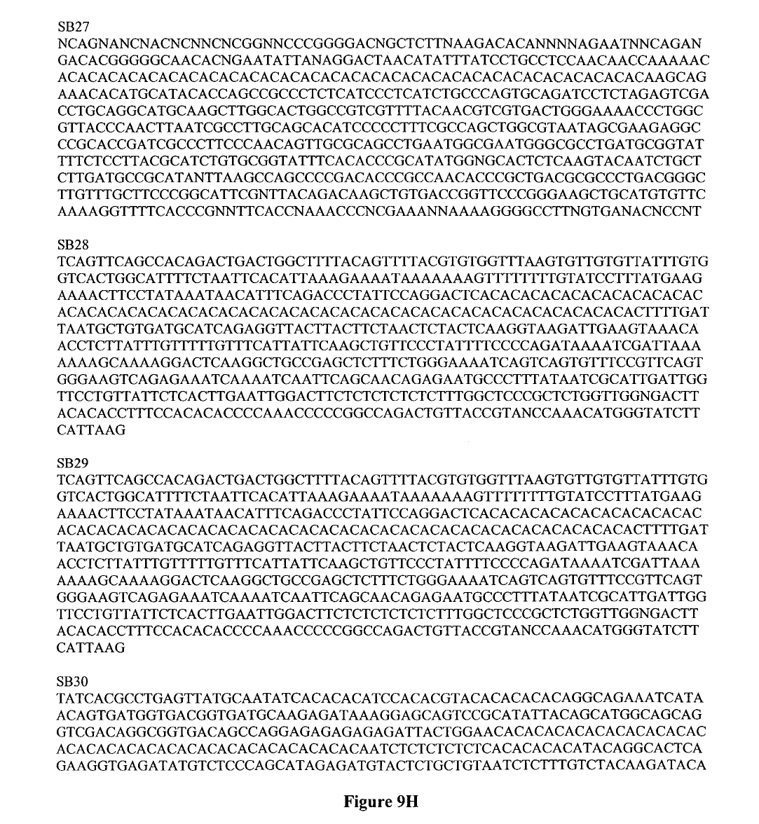

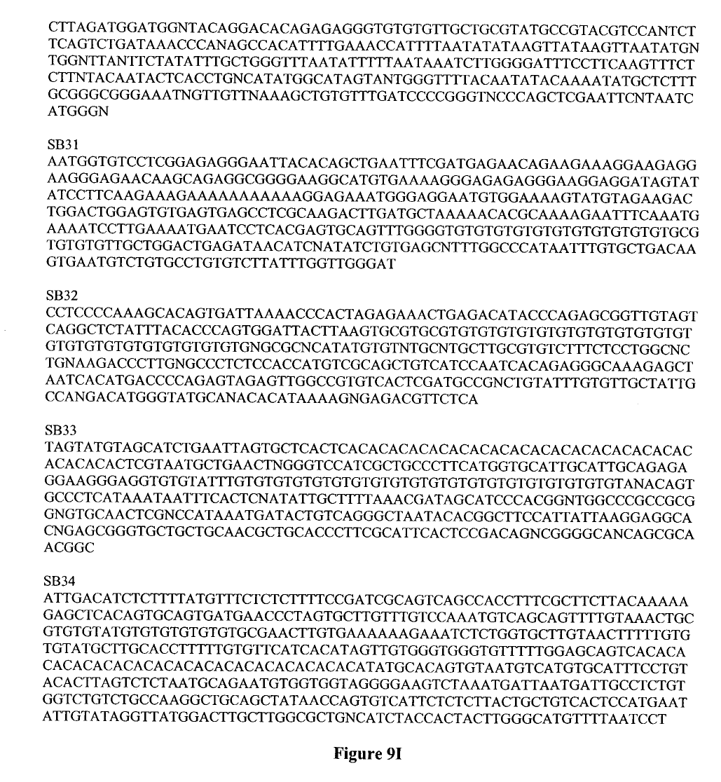

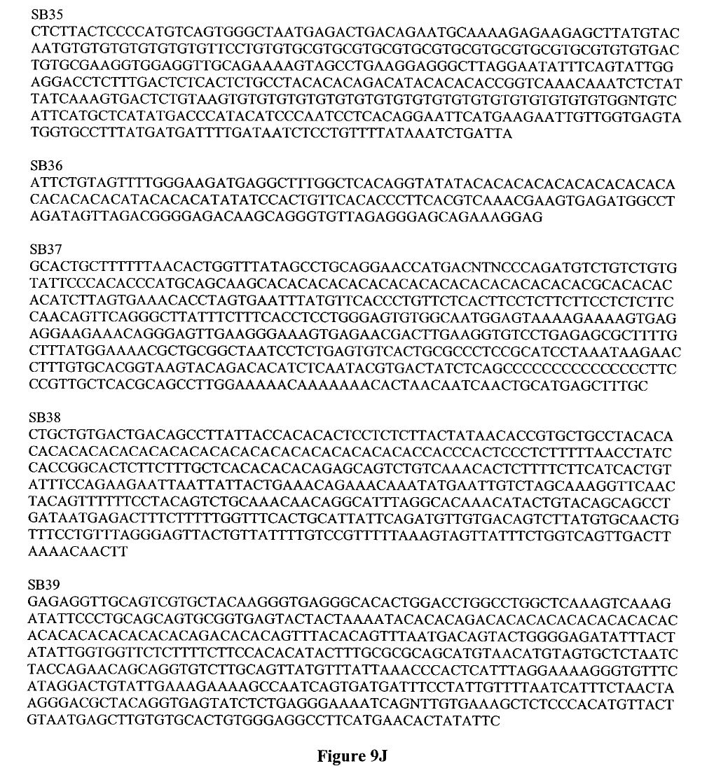

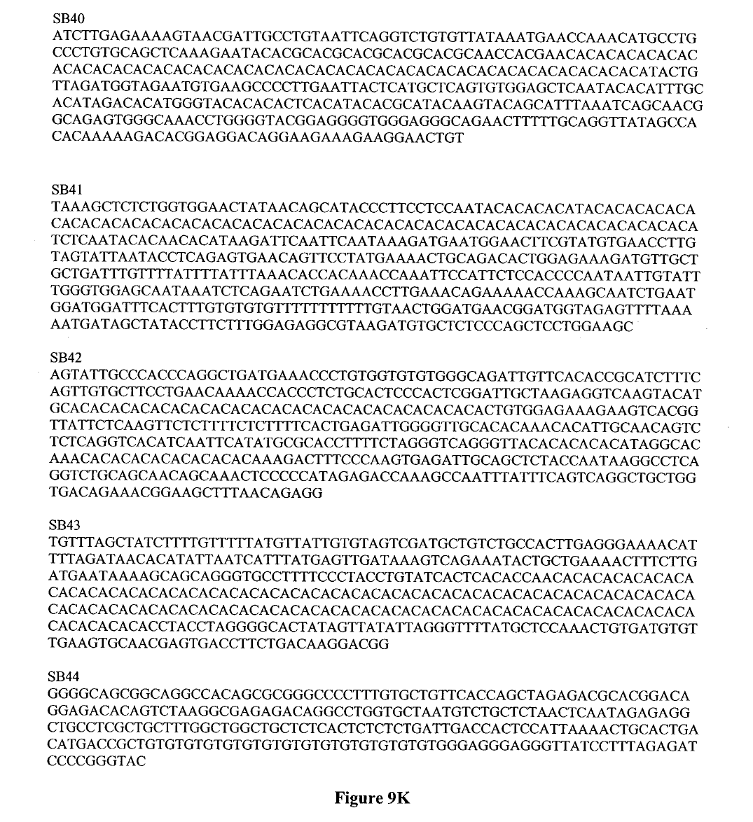

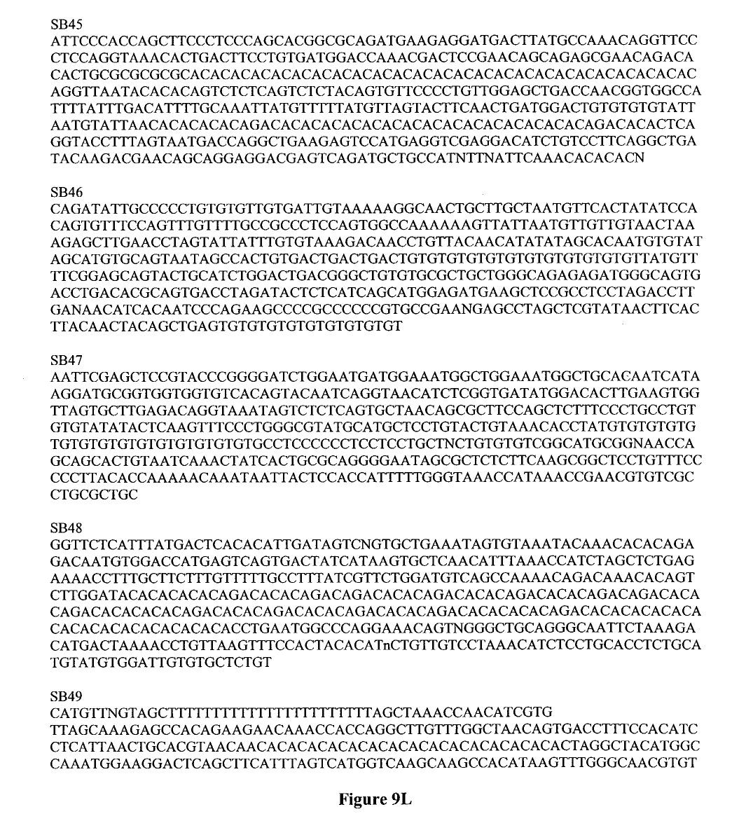

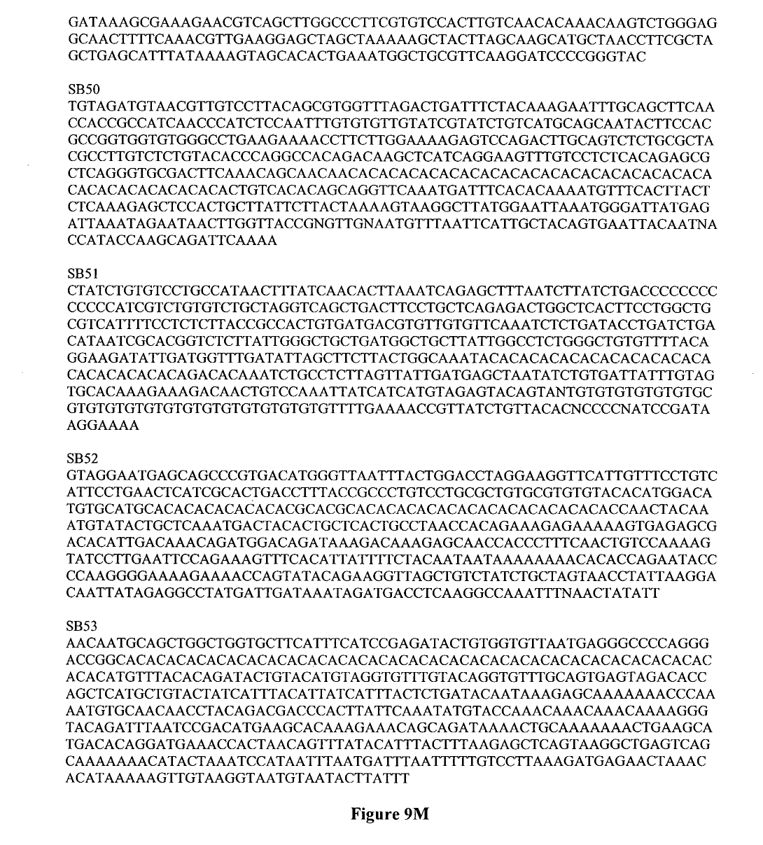

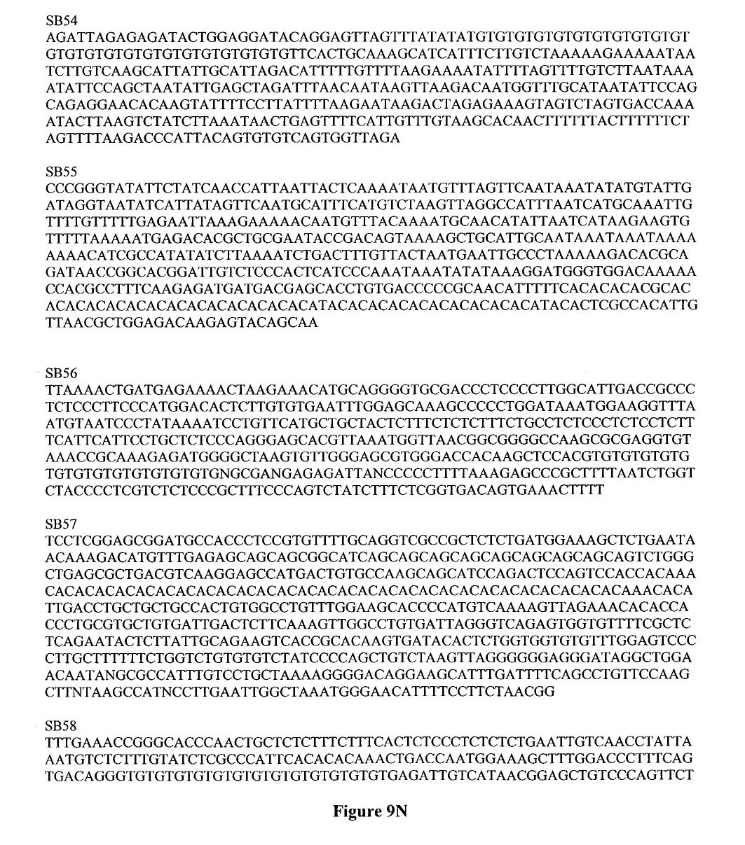

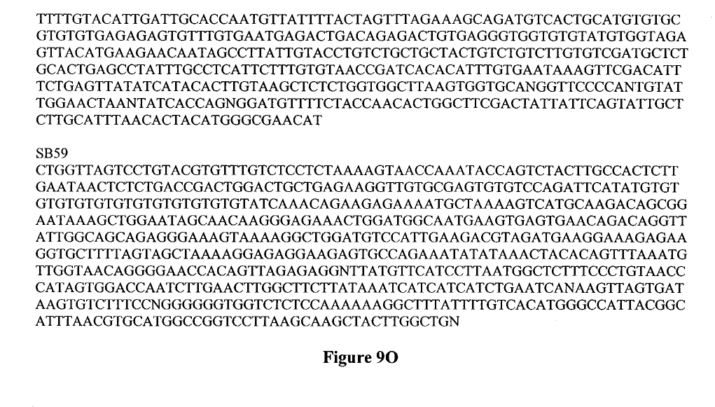

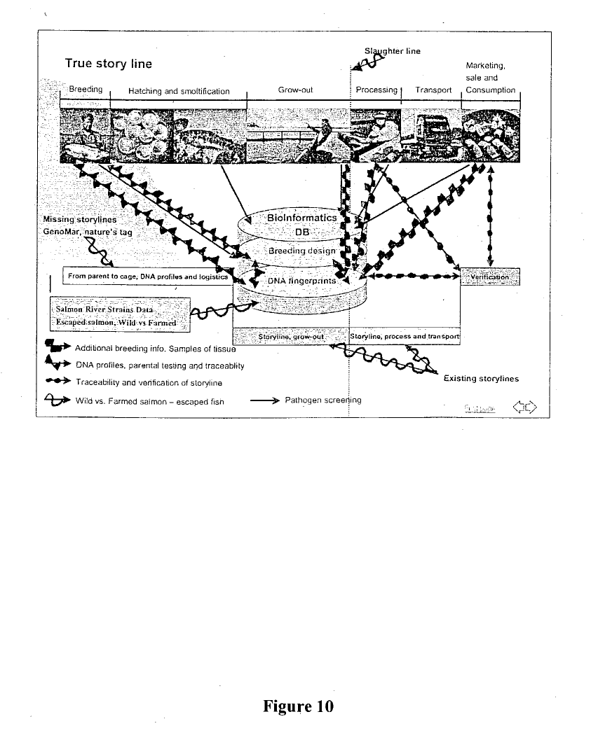

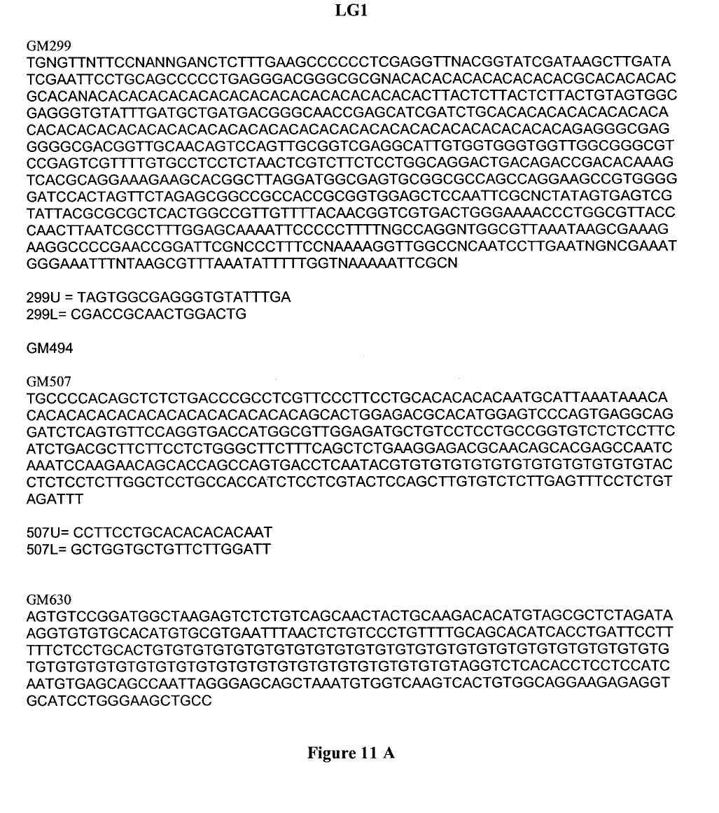

In a further embodiment, the invention provides an isolated nucleic acid molecule encompassing a polymorphic sequence, where the isolated nucleic acid molecule has a nucleotide sequence selected from the group designated SEQ ID NOS: 415-472 and shown in FIG. 9, which correspond to the seabass. Also provided are a nucleic acid molecules that hybridize to the nucleic acid molecule of selected from the group designated SEQ ID NOS: 415-472, or its complement under highly stringent hybridization conditions.

As used herein, the term “seabass” refers to organisms that belong the order Perciformes, the family Serranidae, and include the black sea bass Centropristis, as well as organisms belonging to the family Moronidae, in particular, the European sea bass Dicentrarchus laborax.

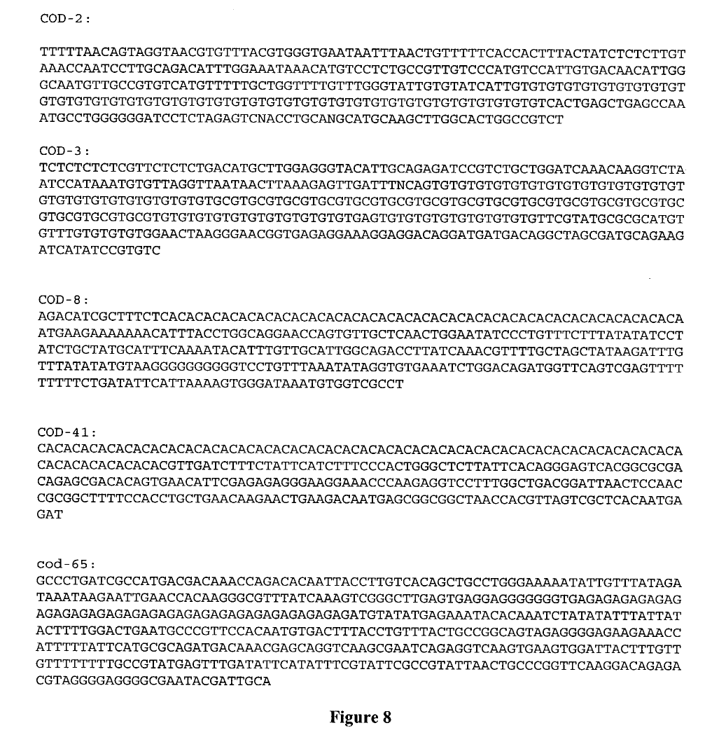

In another embodiment, the invention provides an isolated nucleic acid molecule encompassing a polymorphic sequence, where the isolated nucleic acid molecule has a nucleotide sequence selected from the group designated SEQ ID NOS: 410-414 and shown in FIG. 8, which correspond to cod. Also provided are a nucleic acid molecules that hybridize to the nucleic acid molecule having a nucleotide sequence selected from the group designated SEQ ID NOS: 410-414, or its complement under highly stringent hybridization conditions.

As used herein, the term “cod” refers to the Atlantic cod, which belongs to the order Gadiformes, family Cadidae, species Gadus morhua, and is a saltwater fish found in the North Atlantic above 45° N.

The isolated nucleic acid molecules of the invention encompassing polymorphic nucleotide sequences, including SNPs and microsatellite sequences, as set forth above represent genetic markers that can be used, for example, to genotype fish and are useful as components of a parentage genotype database in the methods of the invention to determine the origin of a fish sample. Furthermore, the invention provides isolated nucleic acid molecules that can be used, for example, as probes to detect the presence of one or more genetic markers in fish samples and in other screening applications known to those skilled in the art.

The invention further is directed to a method of determining the parentage origin of a fish sample by providing a parentage genotype database that contains a collection of candidate parent genotypes, also referred to as candidate origin genotypes, that each represent a distinct parentage origin and comparing a sample genotype to the parentage genotype database, such that a match between a sample genotype and one of the candidate parent genotypes identifies the parentage origin of the sample.

The ability to identify the parentage origin of a fish sample via the methods provided by the present invention allows for improved quality control mechanisms in commercial aquaculture. Genetic markers, for example, an insertion, deletion, rearrangement, single nucleotide polymorphism (SNP), a microsatellite (MS) or a variable number tandem repeat (VNTR) polymorphism, are important tools that allow identification of the parentage origin using the methods provided by the invention. The present invention provides the benefit of allowing direct identification of the parentage individuals or origin population rather than indirect identification merely based on the assignment of a sample to a population based on the matching of genetic profiles based on gene frequencies, a traditionally used method based on the statistical guess that an individual with a specific genetic makeup or genotype belongs to a specific population with a specific gene frequency at those loci. In contrast, the invention method establishes parentage by matching offspring or sample genotype with a set of pre-typed panels corresponding to potential parent or origin genotypes. Thus, the present invention represents a significant improvement over traditional identification methods based on population genetics.

The methods of the invention exemplified herein for an origin or parentage determination of a fish sample are equally applicable to a variety of other organisms and biomaterials. A unique aspect of the invention method, in addition to the particular compositions provided by the invention, is the employment of large-scale parentage or origin analysis based on checking a sample genotype against a parentage or origin genotype database and by that be able to determine which parent pair the particular individual originates from. The invention methods distinguish from traditional tracing systems of livestock, for example, cattle, which is based on individually comparing samples with origin candidates rather than by comparison against an exhaustive origin database.

Due to their high biological capacity for reproduction (fecundity), fish provide an especially appropriate target for practicing the methods of the invention. For example, a female salmon breeder can produce up to 10,000 offspring and some shellfishes have millions of offspring. In particular, genotyping a female salmon breeder and its male partner provides the ability to verify the origin of 40 metric tons of seafood. Regardless of the additional benefits conferred upon the methods of the invention by virtue of the fecundity of fish, the methods are nevertheless also applicable to other biomaterials containing nucleic acid based on the genotyping and subsequent establishment of parentage/origin genotype databases and comparison of a sample genotype against such a database.

The invention further provides an isolated nucleic acid molecule having a nucleotide sequence that hybridizes to a nucleic acid molecule encompassing a polymorphic nucleotide sequence, for example, a SNP and microsatellite sequences of the invention, as set forth in FIGS. 1-9 and 11, or its complement under stringent conditions. In one embodiment, the isolated oligonucleotide comprises at least 17 contiguous nucleotides of a salmon SNP set forth in FIG. 1, or the complement thereof. Such an oligonucleotide is able to specifically hybridize to a complementary nucleic acid molecule under highly stringent hybridization conditions.

Further provided are isolated oligonucleotides containing at least 17 contiguous nucleotides of a SNP-containing nucleic acid molecule or of its complement. Also provided are isolated oligonucleotides containing at least 17 contiguous nucleotides of a microsatellite sequence-containing nucleic acid molecule or of its complement. An isolated oligonucleotide can thus contain at least 18, 19, 20, 22, or at least 25 contiguous nucleotides, such as at least 30, 40, 50, 60, 70, 80, 90, 100, 125, 150, 175, 200, 225, 250, 275, 300, 350, 400, 500, 600, 700, 800 or more contiguous nucleotides from the reference nucleotide sequence, up to the full length sequence. An invention oligonucleotide can be single or double stranded, and represent the sense or antisense strand.

In one embodiment, the isolated oligonucleotide comprises at least 17 contiguous nucleotides of an isolated nucleic acid molecule encompassing a salmon single nucleotide polymorphism (SNP) as described above and set forth in FIG. 1, or the complement thereof. In a further embodiment, the isolated oligonucleotide comprises at least 17 contiguous nucleotides an isolated nucleic acid molecule encompassing a tilapia single nucleotide polymorphism (SNP) as described above and set forth in FIGS. 4 and 6, or the complement thereof. Such oligonucleotides are able to specifically hybridize to a polymorphic nucleic acid molecule of the invention under highly stringent hybridization conditions.

In a further embodiment, the isolated oligonucleotide comprises at least 17 contiguous nucleotides of the microsatellite sequence-containing nucleic acid molecule designated SEQ ID 155-164, or the complement thereof. The invention also provides an isolated oligonucleotide containing at least 17 contiguous nucleotides of the microsatellite sequence-containing nucleic acid molecule designated SEQ ID 309-367, or the complement thereof. In a further embodiment, the isolated oligonucleotide comprises at least 17 contiguous nucleotides of the microsatellite sequence-containing nucleic acid molecules set forth in FIG. 11 (along with corresponding primers) and consecutively designated SEQ ID NOS: 473-1377, or the complement thereof. In a further embodiment, the isolated oligonucleotide comprises at least 17 contiguous nucleotides of the polymorphic sequence-containing nucleic acid molecule designated SEQ ID NOS: 368-373, or the complement thereof. In a further embodiment, the isolated oligonucleotide comprises at least 17 contiguous nucleotides of the polymorphic sequence-containing nucleic acid molecule designated SEQ ID NOS: 374-409, or the complement thereof. In a further embodiment, the isolated oligonucleotide comprises at least 17 contiguous nucleotides of the polymorphic sequence-containing nucleic acid molecule designated SEQ ID NOS: 410-414, or the complement thereof. In a further embodiment, the isolated oligonucleotide comprises at least 17 contiguous nucleotides of the polymorphic sequence-containing nucleic acid molecule designated SEQ ID NOS: 415-472, or the complement thereof. Such oligonucleotides are able to specifically hybridize to a microsatellite sequence-containing nucleic acid molecule under highly stringent hybridization conditions.

The invention oligonucleotides can be advantageously used, for example, as probes to detect polymorphic nucleotide sequence-containing nucleic acid molecules, for example SNP-containing and microsatellite sequence-containing nucleic acid molecules in a sample; as sequencing or PCR primers; or in other applications known to those skilled in the art in which hybridization to a SNP-containing nucleic acid molecule and a microsatellite sequence-containing nucleic acid molecule is desirable.

In one embodiment, the invention provides a primer pair containing an isolated oligonucleotide containing at least 17 contiguous nucleotides of a SNP-containing nucleic acid molecule and an isolated nucleic acid molecule containing at least 17 contiguous nucleotides of the complement of a SNP-containing nucleic acid molecule of the invention. In a further embodiment, the invention provides a primer pair containing an isolated oligonucleotide containing at least 17 contiguous nucleotides of a microsatellite sequence-containing nucleic acid molecule and an isolated nucleic acid molecule containing at least 17 contiguous nucleotides of the complement of a microsatellite sequence-containing nucleic acid molecule of the invention. The primer pairs provided by the invention can be used, for example, to amplify a nucleic acid molecule by the polymerase chain reaction (PCR). The skilled person can determine an appropriate primer length and sequence composition for the intended application.

The present invention further provides isolated nucleic acid molecules encompassing a microsatellite sequence associated with tilapia and set forth as SEQ ID NOS: 309-367 and, set forth along with corresponding primers and consecutively designated as SEQ ID NOS: 473-1377; isolated nucleic acid molecules encompassing a microsatellite sequence associated with Atlantic salmon and set forth as SEQ ID NOS: 155-164; isolated nucleic acid molecules encompassing a polymorphic nucleotide sequence associated with halibut and set forth as SEQ ID NOS: 374-409; isolated nucleic acid molecules encompassing a polymorphic nucleotide sequence associated with cod and set forth as SEQ ID NOS: 410-414; and isolated nucleic acid molecules encompassing a polymorphic nucleotide sequence associated with seabass and set forth as SEQ ID NOS: 415-472. The isolated nucleic acid molecules designated SEQ ID NOS: 155-164, 309-367, 374-472 and those shown in FIG. 11 along with corresponding primers (SEQ ID NOS: 473-1377) encompass polymorphic nucleotide sequences of the above-named species. The invention further provides oligonucleotides that hybridize to the nucleotide sequences of the nucleic acid molecules designated SEQ ID NOS: 155-164, 309-367, 374-472 and those nucleic acid molecules shown in FIG. 11 that correspond to microsatellite sequences, which are consecutively designated with their corresponding primers as SEQ ID NOS: 473-1377.

The term “isolated,” in reference to an invention nucleic acid molecule is intended to mean that the molecule is substantially removed or separated from components with which it is naturally associated, or is otherwise modified by the hand of man, thereby excluding nucleic acid molecules as they exist in nature.

The term “nucleic acid molecule,” as used herein, refers to an oligonucleotide or polynucleotide of natural or synthetic origin. A nucleic acid molecule can be single- or double-stranded genomic DNA, cDNA or RNA, and can represent the sense strand, the antisense strand, or both. A nucleic acid molecule can include one or more non-native nucleotides, having, for example, modifications to the base, the sugar, or the phosphate portion, or having a modified phosphodiester linkage. Such modifications can be advantageous in increasing the stability of the nucleic acid molecule. Furthermore, a nucleic acid molecule can include, for example, a detectable moiety, such as a radiolabel, a fluorochrome, a ferromagnetic substance, a luminescent tag or a detectable binding agent such as biotin. Such modifications can be advantageous in applications where detection of a hybridizing nucleic acid molecule is desired.

As used herein, a “probe” or “oligonucleotide” is single-stranded or double-stranded DNA or RNA, or analogs thereof, that has a sequence of nucleotides that includes at least 15, at least 20, at least 50, at least 100, at least 200, at least 300, at least 400, or at least 500 contiguous bases that are the same as, or the complement of, any contiguous bases set forth in any of SEQ ID NOS: 1-1377. oligonucleotides are useful, for example, as probes or as primers for amplification reactions such as the polymerase chain reaction (PCR). In addition, oligonucleotides can bind to the sense or anti-sense strands of other nucleic acids. Preferred regions from which to construct a probe include those nucleic acid sequences that contain the SNP or a microsatellite. Probes can be labeled by methods well-known in the art, as described hereinafter, and used in various diagnostic kits.

As used herein, the term “single nucleotide polymorphism” or “SNP” is intended to mean a difference in nucleotide sequence between two related nucleic acid molecules of one nucleotide at a specified position. The term refers to a nucleotide substitution at a particular position compared to an otherwise identical nucleic acid sequence at adjacent nucleotide positions. Therefore, the term refers to a relative difference in primary structure between two compared nucleic acid molecules that are substantially related.

As used herein, the term “microsatellite” or “microsatellite sequence” is intended to refer to a tandem repeat sequence that is either present or varies in length at a particular position compared to an otherwise identical nucleic acid sequence at the same nucleotide positions.

The term “polymorphic” as used herein to a nucleotide sequence of the invention is intended to refer any variation in nucleotide sequence between two related nuclear acid molecules and is meant to encompass both SNPs and microsatellites.

Eucaryotic genomes contain a large number of single nucleotide polymorphisms, which make it easy to look for allelic versions of a gene by sequencing samples of the gene taken from different members of a population or from a heterozygous individual. Similarly, eucaryotic genomes contain a large number of interspersed simple tandem repeat sequences, designated microsatellites, which vary in length among individuals. SNPs and microsatellites represent highly informative polymorphic markers that can be typed, for example, using the polymerase chain reaction (PCR). Such polymorphic sequence variants further can be detected using the oligonucleotide ligation assay (OLA) as described in Example 2, or other appropriate detection method known in the art.

The invention nucleic acid molecules and oligonucleotides can be advantageously used, for example, as probes to detect nucleic acid molecules encompassing a particular single nucleotide polymorphism in a sample; as probes to detect nucleic acid molecules encompassing a particular microsatellite sequence in a sample; as sequencing or PCR primers; or in other applications known to those skilled in the art in which hybridization to an invention nucleic acid molecule is desirable.

Hybridization refers to the binding of complementary strands of nucleic acid, for example, sense:antisense strands or probe:target-DNA, to each other through hydrogen bonds, similar to the bonds that naturally occur in chromosomal DNA. Stringency levels used to hybridize a given probe with target-DNA can be readily varied by those of skill in the art.

Stringent hybridization are conditions under which polynucleic acid hybrids are stable. As known to those of skill in the art, the stability of hybrids is reflected in the melting temperature (Tₘ) of the hybrids. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

Specific hybridization refers to the ability of a nucleic acid molecule to hybridize to the reference nucleic acid molecule without hybridization under the same conditions with nucleic acid molecules that are not the reference molecule. Under moderately stringent hybridization conditions the hybridized nucleic acids will generally have at least about 60% identity, at least about 75% identity, more at least about 85% identity; or at least about 90% identity. Moderately stringent conditions are conditions equivalent to hybridization in 50% formamide, 5× Denhart's solution, 5×SSPE, 0.2% SDS at 42□C, followed by washing in 0.2×SSPE, 0.2% SDS, at 42□C. In contrast, high stringency hybridization conditions can be provided, for example, by hybridization in 50% formamide, 5× Denhart's solution, 5×SSPE, 0.2% SDS at 42□C, followed by washing in 0.1×SSPE, and 0.1% SDS at 65□C. Low stringency hybridization conditions include hybridization in 10% formamide, 5× Denhart's solution, 6×SSPE, 0.2% SDS at 22□C, followed by washing in 1×SSPE, 0.2% SDS, at 37□C. Denhart's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderately stringent and highly stringent hybridization buffers and conditions are well known to those of skill in the art and are described, for example, in Sambrook et al., Molecular Cloning: A Laboratory Manual, 3rd ed., Cold Spring Harbor Press, Plainview, N.Y. (2001) and in Ausubel et al. (Current Protocols in Molecular Biology (Supplement 47), John Wiley & Sons, New York (1999)).

Nucleic acid molecules of the invention hybridize under moderately stringent or highly stringent conditions to substantially the entire sequence, or substantial portions, for example, typically at least 15, 17, 21, 25, 30, 40, 50 or more nucleotides of the nucleic acid sequence set forth in SEQ ID NOS: 1-1377.

An invention nucleic acid molecule or oligonucleotide containing a single nucleotide polymorphism or a microsatellite sequence can further contain nucleotide additions or additional nucleotide sequences including, for example, sequences that facilitate identification of the oligonucleotide.

The invention also provides an isolated nucleic acid probe that specifically hybridizes to and detects a polymorphic nucleic acid sequence of the invention, wherein the polymorphic nucleic acid sequence is selected from nucleic acid molecules set forth, along with corresponding primers, in FIGS. 1-9 and 11 and designated SEQ ID NOS: 1-1377. Therefore, the invention provides an isolated nucleic acid probe that specifically hybridizes to and detects nucleic acid sequence encompassing a SNP or microsatellite sequence as described herein. An isolated nucleic acid probe of the invention contains at least approximately 17 contiguous nucleotides of the complement of a polymorphic nucleic acid molecule of the invention. The probe can be used, for example, to detect the presence of a SNP-containing nucleic acid molecule in a sample. The skilled person can determine an appropriate probe length and sequence composition for the intended application.

The invention provides an isolated nucleic acid probe that specifically hybridizes to and detects nucleic acid sequence encompassing a SNP, wherein the nucleic acid sequence is selected from the group shown in FIG. 1 along with primer sequences as SEQ ID NOS: 1-112. The invention further provides an isolated nucleic acid probe that specifically hybridizes to and detects nucleic acid sequence encompassing a SNP, wherein the nucleic acid sequence is selected the group shown in FIG. 4 along with primer sequences as SEQ ID NOS: 165-308. The invention further provides an isolated nucleic acid probe that specifically hybridizes to and detects nucleic acid sequence encompassing a SNP, wherein the nucleic acid sequence is selected the group shown in FIG. 7 and designated SEQ ID NOS: 374-409.

The invention further provides an isolated nucleic acid probe that specifically hybridizes to and detects a polymorphic nucleic acid sequence, wherein the nucleic acid sequence is selected the group shown in FIGS. 6, 8 and 9; set forth as SEQ ID NOS: 368-373 and 410-472. The invention also provides an isolated nucleic acid probe that specifically hybridizes to and detects nucleic acid sequence encompassing a microsatellite sequence, wherein the nucleic acid sequence is selected from the group shown in FIGS. 3 and 5; set forth as SEQ ID NOS:155-164 and 309-367. The invention further provides an isolated nucleic acid probe that specifically hybridizes to and detects nucleic acid sequence encompassing a microsatellite sequence, wherein the nucleic acid sequence is selected the group shown in FIG. 11 along with primer sequences as SEQ ID NOS: 473-1377. As described herein, an isolated nucleic acid probe of the invention contains at least approximately 17 contiguous nucleotides of the complement of a SNP-containing nucleic acid molecule of the invention or a microsatellite-containing nucleic acid molecule of the invention. The probe can be used, for example, to detect the presence of a SNP-containing nucleic acid molecule or a microsatellite-containing nucleic acid molecule in a sample. The skilled person can determine an appropriate probe length and sequence composition for the intended application.

An isolated nucleic acid molecule or oligonucleotide of the invention can be produced or isolated by methods known in the art. The method chosen will depend, for example, on the type of nucleic acid molecule one intends to isolate. Those skilled in the art, based on knowledge of the nucleotide sequences disclosed herein, can readily isolate the isolated nucleic acid molecules as genomic DNA; as full-length cDNA or desired fragments therefrom; or as full-length mRNA or desired fragments therefrom, by methods known in the art.

An invention nucleic acid molecule does not consist of the exact sequence of a nucleotide sequence set forth in publically available databases, such as Expressed Sequence Tags (ESTs), Sequence Tagged Sites (STSs) and genomic fragments, deposited in public databases such as the nr, dbest, dbsts and gss databases, and TIGR, SANGER center, WUST1 and DOE databases.

One useful method for producing an isolated nucleic acid molecule of the invention involves amplification of the nucleic acid molecule using the polymerase chain reaction (PCR) and specific primers and, optionally, purification of the resulting product by gel electrophoresis. Either PCR or reverse-transcription PCR (RT-PCR) can be used to produce a nucleic acid molecule having any desired nucleotide boundaries. Desired modifications to the nucleic acid sequence can also be introduced by choosing an appropriate primer with one or more additions, deletions or substitutions. Such nucleic acid molecules can be amplified exponentially starting from as little as a single gene or mRNA copy, from any cell, tissue or species of interest.

Furthermore, an isolated nucleic acid molecule or oligonucleotide of the invention can be produced by synthetic means. For example, a single strand of a nucleic acid molecule can be chemically synthesized in one piece, or in several pieces, by automated synthesis methods known in the art. The complementary strand can likewise be synthesized in one or more pieces, and a double-stranded molecule made by annealing the complementary strands. Direct synthesis is particularly advantageous for producing relatively short molecules, such as oligonucleotide probes and primers, and nucleic acid molecules containing modified nucleotides or linkages.

Genetic markers, for example, an insertion, deletion, rearrangement, SNP, microsatellite or variable number tandem repeat (VNTR) polymorphism, are important tools that allow identification of the parentage origin using the methods provided by the invention. For example, the presence in a fish sample of a nucleic acid molecule of the invention containing a polymorphic nucleotide sequence, for example, a SNP or a microsatellite sequence is indicative of the origin of the sample. Thus, the invention provides methods for detecting a nucleic acid molecule containing a SNP or a microsatellite in a fish sample. This information can be useful, for example, to determine the origin of the fish sample.

In one embodiment, the method is practiced by contacting a sample containing nucleic acids with one or more oligonucleotides containing contiguous sequences from a SNP-containing nucleic acid molecule of the invention, under high stringency hybridization conditions, and detecting a nucleic acid molecule that hybridizes to the oligonucleotide. In an alternative embodiment the method is practiced by contacting a fish sample with a primer pair suitable for amplifying a SNP-containing nucleic acid molecule of the invention, amplifying a nucleic acid molecule using polymerase chain reaction, and detecting the amplification.

As used herein, the term “sample” is intended to mean any biological fluid, cell, tissue, organ or portion thereof, or any environmental sample (e.g. soil, food, water, effluent and the like) that contains or potentially contains a SNP-containing nucleic acid molecule of the invention. For example, a sample can be an egg, a section obtained from a commercially sold fish filet, breeder, smelt, slaughtered fish, or can be a subcellular fraction or extract, or a crude or substantially pure nucleic acid preparation. A sample can be prepared by methods known in the art suitable for the particular format of the detection method employed. A sample can correspond to an individual fish or can correspond to more than one individual.

The methods of detecting a nucleic acid molecule in a sample can be either qualitative or quantitative, and can detect the presence, abundance, integrity or structure of the nucleic acid molecule as desired for a particular application. Suitable hybridization-based assay methods include, for example, in situ hybridization, which can be used to detect altered chromosomal location of the nucleic acid molecule, altered gene copy number, and RNA abundance, depending on the assay format used. Other hybridization methods include, for example, Northern blots and RNase protection assays, which can be used to determine the abundance and integrity of different RNA splice variants, and Southern blots, which can be used to determine the copy number and integrity of DNA. A hybridization probe can be labeled with any suitable detectable moiety, such as a radioisotope, fluorochrome, chemiluminescent marker, biotin, or other detectable moiety known in the art that is detectable by analytical methods.

Suitable amplification-based detection methods are also well known in the art, and include, for example, qualitative or quantitative polymerase chain reaction (PCR); reverse-transcription PCR (RT-PCR); single strand conformational polymorphism (SSCP) analysis, which can readily identify a single point mutation in DNA based on differences in the secondary structure of single-strand DNA that produce an altered electrophoretic mobility upon non-denaturing gel electrophoresis.

The invention also provides a method of determining the origin of a fish sample by providing a parentage genotype database encompassing a collection of candidate parent genotypes, wherein each of the candidate parent genotypes represents a distinct parent; and comparing a sample genotype to the parentage genotype database, wherein a match between the sample genotype and one of the candidate parent genotypes or the genotype of each of the two individuals in a parent pair identifies the origin of the sample.

In a related but distinct embodiment, the invention provides a method of determining the origin of a fish sample by providing an origin genotype database encompassing a collection of candidate genotype profiles, wherein each of the candidate genotype profiles represents a distinct population of origin; and comparing a sample genotype to the candidate genotype profiles, wherein a match between the sample genotype and one of the candidate genotype profiles identifies the population of origin of the sample.

The terms “parentage genotype database” and “origin genotype database” as used herein, refer to a compilation of a collection of nucleotide sequences corresponding to candidate parent genotypes or candidate genotype profiles, respectively, in a centralized location that is capable of being searched with a sample gentoype to determine a match.

As used herein, the terms “candidate parent genotype” and “candidate origin genotype,” refer to the individual components of the collection that make up the “parentage genotype database” or “origin genotype database,” respectively. The concept of an origin versus a parent can be used to include the situation where the database includes individuals removed by more than one generation as well as to include other databases encompassing genotypes that do not correspond to parent components, for example, those comprised of biomaterials not capable of sexual reproduction. In addition, an origin genotype consist of a profile or panel that reflects a genetically unique set of markers corresponding to a specific population or batch rather than an individual parent as is desired in those embodiments where the method is practiced to identify, for example, a sample, for example, a fingerling, with regard to a distinct genetic combination of potential parents. The unique spectrum of genetic profiles created by a particular parent population or population of origin can thus be used to trace a sample to a specific producer.

As used herein, the term “origin” refers to the source that is identified by matching the genotype of a sample to a collection of candidate genotypes consisting of, for example, individual candidate parent genotypes or candidate genotype profiles/panels. As described herein, in certain embodiments of the invention it is desirable to identify the originating broodstock in a strict parentage test, while in other embodiments the invention methods can be utilized to match a candidate to a population of origin or batch of origin that is represented by a genotype profile or panel that collectively reflects a group of individual parents.

The parentage or origin genotype database encompasses a collection of candidate parent or origin genotypes, which can be established through genetic markers known in the art and described herein, for example, those represented by the SNPs and microsatellite sequences encompassed in the nucleic acid molecules provided by the invention. The genetic markers are sufficient to distinguish one of the candidate parent genotypes from other candidate parent genotypes in the database. The parentage genotype database can comprise genotypes of 2 or more, 3 or more, 5 or more, 10 or more, 20 or more, 50 or more, 100 or more, 200 or more, 500 or more, 1000 or more, 2000 or more, 5000 or more, or 10,000 or more, 15,000 or more, 30,00 or more, or 60,000 or more candidate parents. In addition, the number of genetic markers required to obtain the required statistical power to practice the methods of the invention depends on a variety of factors, including, the desired application of the method, the allele frequency of the marker, and the size of the collection encompassing the database. It is contemplated that at least 30 or more, at least 40 or more, at least 50 or more, at least 60 or more, at least 70 or more, at least 80 or more, at least 90 or more, at least 100 or more, at least 120 or more SNPs must be typed in methods for assigning a parent pair via the invention methods. In addition, it is estimated that at least 5 or more, at least 10 or more, at least 20 or more, at least 40 or more microsatellite markers can be typed in methods for assigning an individual to a parent pair via the invention methods. It is understood that the number of markers necessary can based on the particular parameters provided by the breeding and production organization, for example, different numbers of families in the production units.

In a preferred embodiment, the parentage genotype database is exhaustive, which means it can include all of the candidate parent or origin genotypes that potentially could represent the parental origin of a sample. For example, a parentage genotype database can include genotypes of substantially all of the parents from each hatchery that provides fingerlings. The number of candidate parent or origin genotypes in a parentage genotype database will depend on the needs of the user and will vary depending on the source of the sample to be identified, the availability of access to candidate parent or origin genotypes and the complexity of genetic markers expressed in the sample.

The parentage or origin genotype database can be directed to a candidate parent or origin genotypes of a single species or can contain representative genotypes corresponding to a variety of potential origin species, for example, cod and tilapia, as desired. Species specific markers may be used in order to verify or test whether a food sample or individual sample represents the species that the sample is sold or marketed as.

In one embodiment, the invention can be practiced to verify species origin. In this regard, the total result of genotyping of a sample with a high number of markers can be used to verify that the sample belongs to a species based on predetermined information regarding markers, which can be supplied, for example, by the producer. Although a proportion markers may correspond more than one species, differences in the number of alleles, allele sizes and allele frequencies can be used to distinguish between species.

Furthermore, if desired by the user, the candidate parent or origin genotypes can represent, for example, two populations such as farm raised salmon and wild salmon and the invention used to assign a sample to one of these candidate populations of origin rather than to a particular parent pair.

Thus, the invention provides a parentage or origin genotype database encompassing a collection of candidate parent or origin genotypes. The candidate parent or origin genotypes can be constructed by a variety of genotyping methods known those skilled in the art and described herein, for example, using genetic markers provided by the present invention.

It is contemplated that the parentage genotype database can encompass genotypes of existing broodstock and can be a complete collection of all broodstock genotypes. It is contemplated that the highest possible number of breeders from the hatcheries supplying samples is genotyped for inclusion in the parentage genotype database. In addition to genotyping broodstock and for optional inclusion into the parentage genotype database, it is further contemplated that genotyping can also be performed for a representative number of individuals from a farm when smelt are introduced or fish are harvested for slaughter. Thus, in the methods of the invention a parentage genotype database can be a partial or complete collection of candidate parent or origin genotypes corresponding to a desired population of potential parents. Once determined, the sample genotype can be compared to the parentage origin database.

It is understood that the methods provided by the invention enable the user to trace back not only to the individual genetic origin, for example, as defined by broodstock, breeding nucleus or hatchery, but also can be used to trace a sample back to any level desired throughout the food value chain by selecting the appropriate markers. This embodiment of the invention, which also can be described as optimized genetic logistics or genetic flow control, is predicated on the fact that a member of the production system, for example, a farmer receives distinct and identifiable batches of genetic material from the broodstock of origin or from the last multiplier providing seeds to the farmer such that the parents giving rise to the sample can be identified, typed and used to establish a genotype profile or panel. In particular, although a farmer may share genetic material with other farmers, each farmer receives a unique set of fingerlings originating from a distinct combination of parents that do not give rise to offspring in other farms—if the distribution from the hatcheries is organized optimally. Therefore, the provider of fingerlings, generally the hatchery, can collect and type DNA corresponding to different sets of parents that will give rise to specific batches of offspring targeting different farmers. In this embodiment of the invention methods, not only the brood stock profile as defined by its parentage genotype collection will be unique, but every farmed fish population or batch will be assigned a unique genetic origin genotype profile or panel that allows tracing the population of origin.

The methods of the invention for determining the origin of a fish sample by providing an origin genotype database encompassing a collection of candidate genotype profiles or panels further enable an individual producer or entity within the commercial chain, for example, a farmer, to collect tissue from a representative number of the fish traded, for example, to be traded at the wholesale level, and establish a “biobank,” which is another term for an origin genotype database that encompasses a collection of candidate genotype profiles. Once established, the biobank can be accessed to either verify the origin of a particular sample or exclude the corresponding producer as a potential source of the sample, for example, in situations of pathogen contamination, irregular or illegal acts.

Thus, as described herein, the invention methods allow for tracing a food sample back virtually to any level of the commercial chain by utilizing unique genetic markers and instant verification technology against a comprehensive or exhaustive database. The methods involve parentage or origin tests at different levels and can further be combined with other methods known in the art, for example, matching of genetic profiles based on gene frequencies, a method that relies on the statistical likelihood that an individual with a specific genetic makeup or genotype belongs to a specific population with a specific gene frequency at those loci. By comparison, the invention methods identify origin or parentage on the basis of direct matching of the offspring or sample genotype with a collection of genotypes that represent individual parentage or genotype profiles or panel reflecting a unique population of origin. A biobank further can encompass mitochondrial genetic markers that are useful in the methods for identifying parentage or origin based on their maternal inheritance pattern.

The determination of the genotypes corresponding to the sample as well as to the collection of candidate parent or origin genotypes that make up the origin database can be accomplished by a variety of genotyping methods known in the art and described herein and can utilize a variety of genetic markers, including, for example, the particular SNP and microsatellite markers provided by the invention. Thus, a parentage genotype database, which can be constructed to contain a collection of candidate parent or origin genotypes can be accessed by a variety of means to compare a sample genotype and determine its origin/parentage. The determination of the sample genotype can be performed instantaneously, for example, using array or chip technology known in the art and the results can be advantageously transmitted via satellite or via a computer, allowing direct or remote linking to a central repository containing the origin genotype database by methods disclosed herein.

In a preferred embodiment of the invention method, the genotype determination of the candidate parent or origin genotypes that make up the parentage genotype database is performed via an accurate and fast high-throughput method, for example, a chip-based or gel-based method for detecting poymorphic markers, such as, for example, the SNPs or microsatellite sequences provided by the invention set forth in FIGS. 1-9 and 11 along with corresponding primer sequences (SEQ ID NOS: 1-1377). Because of the large number of individuals that will be genotyped for inclusion in the parentage genotype database, it is important that the genotyping system employed is appropriate for high-throughput conditions. In particular, genotyping methods that avoid multiple steps and do not require, for example, performance of PCR or electrophoresis are particularly useful for genotyping candidate origin or parent individuals. For example the Invader□ detection platform, which involves direct hybridization of genomic DNA with differentially labelled SNP-containing probes allows sensitive and accurate detection of SNPs without sample amplification by PCR, as well as other technologies known in the art for fast and accurate high-throughput genotyping are useful in the methods of the invention.

The methods of the invention thus can employ a variety of genotyping methods available for characterization of genetic variation including, for example, techniques based arrays, solution-based, bead-based and gel-based systems, and MALDI-TOF mass spectrometry. Arrays, which involve binding of the sample molecules to a target on a substrate, can comprise a glass slide, or a semi-solid substrate, such as nitrocellulose membrane and the sample nucleotide sequence can be DNA, RNA, or any permutation thereof. One convenient method for determining the sample genotype involves use of a micoarray.

In contrast to the genotyping of the candidate parent genotypes that make up the parentage genotype database, different criteria are of importance in the selection of a genotyping method for the sample. As described herein, the methods of the invention can involve remote methods in which the step of determining the sample genotype is physically separated from the step of comparing the sample genotype to the parentage genotype database. For example, the sample genotyping can be performed by an individual with a low level expertise at a remote location, such as a warehouse, store, or anywhere along the commercial chain. Therefore, it is understood that the sample genotyping is approriately performed via a reliable, robust and relatively simple methodology, for example, a chip technology such as the Motorola eSensor□ DNA chip system. It is contemplated that capturing probes for the SNPs, for example, nucleic acid molecules of the invention as described herein, are placed at the surface of the chip and hybridized to a pool of PCR products representing the profiling nucleic acid molecules. Subsequently, a second hybridization can be performed using differentially labelled probes, for example, oligonucleotide probes provided by the present invention and described herein. Upon application of a slight voltage to the chip, electronic signals will communicate the particular SNPs detected in the sample and, thereby, the sample genotype.

As described herein, the methods of the invention can be used in direct methods performed at any point along the production line between hatchery and consumer. Therefore, the sample can be an egg as well as a filet sample corresponding to a findling or any other sample appropriate for gentoyping. The nucleic acid material to be genotyped can be extracted by any method desired by the user including, for example, automated extraction using a commercially available isolation robot. In a preferred embodiment, the methods of the invention can be used in remote methods in which the step of determining the sample genotype is physically separated from the step of comparing the sample genotype to the parentage genotype database. For example, the sample genotyping can be performed by a sales employee at a remote location, such as a warehouse, store, or anywhere along the commercial chain, and the comparison step performed instantaneously at a different location by conveniently interfacing the remote locations via a network such as the internet.

Once a sample genotype has been determined it is contemplated that origin determination can be performed instantenously. If desired, a parentage genotype database can be conveniently stored on a computer readable medium. Accordingly, the invention provides a computer readable medium encompassing an parentage genotype database, for example, an exhaustive collection of candidate parent or origin genotypes. Such a computer readable medium encompassing a parentage genotype database is useful for comparing the sample gentype with the candidate parent or origin genotypes, which can be conveniently performed on a computer apparatus. The use of a computer apparatus is convenient since a parentage genotype database can be conveniently stored and accessed for comparison to the genotype of a sample. A parentage genotype database can be conveniently accessed using appropriate hardware, software, and/or networking, for example, using hardware interfaced with networks, including the internet. By using various hardware, software and network combinations, the methods of the invention including the step of comparing the genotype of a sample to a parentage genotype database can be conveniently performed in a variety of configurations. Accordingly, the invention additionally provides a computer apparatus for carrying out computer executable steps corresponding to steps of invention methods. For example, a single computer apparatus can contain instructions for carrying out the computer executable step(s) of comparing the genotype determined for a sample to a parentage genotype database, and instructions for determining whether the sample genotype corresponds to one or more of the candidate parent or origin genotypes in the parentage genotype database.

Alternatively, the computer apparatus can contain instructions for carrying out the steps of an invention method while the parentage genotype database is stored on a separate medium. In addition, instructions for determining whether a sample genotype corresponds to candidate parent or origin genotypes in the parentage genotype database can be contained on a separate computer apparatus or separate medium, or combined with the computer apparatus containing the computer executable steps of the method and/or the database on a separate medium. Such a separate computer readable medium can be another computer apparatus, a storage medium such as a floppy disk, Zip disk or a server such as a file-server, which can be accessed by a carrier wave such as an electromagnetic carrier wave. Thus, a computer apparatus containing a parentage genotype database or a file-server on which the parentage genotype database is stored can be remotely accessed, for example, via a satellite or via a network such as the internet. One skilled in the art will know or can readily determine appropriate hardware, software or network interfaces that allow interconnection of an invention computer apparatus.

A parentage genotype database useful in the methods of the invention is interactive and capable of being updated with additional candidate parent or origin genotypes. It further is contemplated that the database includes the appropriate software providing statistical algorithms that can be implemented directly to compare the sample genotype to the collection of candidate parent gentoypes without having to resort to transferring data to a further location. Routines for the estimation of likelihood of origin of a sample are well known in the art and include, for example, Maximum Likelihood, Quasi-Maximum Likelihood and Generalized Method of Moments.

While the invention method is exemplified for fish species and fish/seafood products, those skilled in the art will appreciate that the methods provided by the invention are applicable to identify other species by genotyping samples and comparison of the sample genotypes with genotypes of potential parents. Thus, the invention method is applicable to plant and animal species that have a reproduction method similar to, for example, tilapia, salmon and other fish species, in particular, involving the mating of two parents in order to produce a set of offspring.

It is understood that modifications which do not substantially affect the activity of the various embodiments of this invention are also included within the definition of the invention provided herein. Accordingly, the following examples are intended to illustrate but not limit the present invention.

### EXAMPLE 1

### Isolation of SNP Markers from Salmon and Tilapia

This example describes isolation of genomic DNA containing SNP markers from an Atlantic salmon (Salmo salar) individual and a Nile tilapia (Oreochromis niloticus) individual.

Two genomic libraries, one for tilapia and one for salmon were constructed using the following procedure. The genomic DNA was digested with restriction enzyme Sau 3A (Gibco BRL) followed by electrophoresis in a 1% TBE agarose gel. Using 1 Kb DNA size ladder (Amersham Pharmacia), DNA fragments of the size range 900-1100 bp were excised from the gel and isolated using QIAquick Gel extraction kit (Qiagen). The isolated DNA fragments were then ligated to Ready-to-Go pUC18 (Amersham Pharmacia), linearized with BamHI, BAP treated and formulated with T4 DNA ligase, followed by transformation into E. coli Pack Gold supercompetent cells (Stratagene). Cells from the libraries were grown on LA amp agar plates and clones were picked at random and cultured over night in LB medium. Plasmids were then isolated using QIAprep Spin Miniprep kit (Qiagen) followed by sequencing of the clone insert using standard M13 forward and reverse sequencing primers and Big Dye Terminator Sequencing kit (ABI).

Primers for PCR were designed from the insert sequences seeking to obtain as large amplicons as possible and with a minimum length of 400 bp using the Primer3 software (http://www-genome.wi.mit.edu/cgi-bin/primer/primer3_www.cgi). Primers were ordered from and synthesized at MWG, Germany, and an additional M13 forward (5′-TGT AAA ACG ACG GCC AGT-3′) or reverse (5′-CAG GAA ACA GCT ATG ACC-3′) sequence was added to the 5′ end of each forward and reverse prime respectively in each PCR primer pair in order to simplify subsequent sequencing efforts.

Using the PCR primers described above amplicons were produced from six DNA samples: individually genomic DNA samples from five unrelated fishes as well as a sample of pooled DNA from 20 fish. The PCR reaction took place in a total volume of 20 μl, consisting of 100 ng DNA, 5 pmol of each primer, 2 μl dNTP (2 mM), 2 μl 10×PCR buffer (supplied by ABI optimized for the enzyme), 0.2 μl Ampli-taq polymerase (ABI). Temperature cycling was performed with an initial denaturation step of 95° C. for 3 minutes, then 12 cycles of 95° C. for 30 seconds each, 58° C. for 30 seconds and 72° C. for 30 seconds, then 25 cycles at 95° C. for 30 seconds each and 68° C. for 1 minute. Amplification was performed on a GeneAmp 9600 from ABI.

Subsequent to performance of the PCR, 3.6 μl PCR-product was mixed with 0.7 μl Exonuclease I (10 U/μl Amersham) and 0.7 μl Shrimp Alkaline Phosphatase (2 U/μl Amersham) and incubated at 37° C. for 15 min followed by 80° C. for 15 minutes.

The purified PCR segments were sequenced with the Big Dye Terminator-kit from ABI following the supplied recommended protocol, with standard M13 forward and reverse primers matching the respective sequences at the primer ends of the amplicon and analysed on an ABI 377 Automated Sequencer from ABI. The DNA sequences from the 5 individuals and the DNA pool were aligned using Sequencher™ 4.1 software (Gene Codes Corporation, USA) and SNPs were identified as irregular point variations.

### EXAMPLE 2

### Determination of SNP Variation in Tilapia and Salmon

This example describes the analysis of tilapia and salmon SNPs by oligonucleotide ligation assay (OLA).

The three primers of a OLA analysis were designed as follows:
1) Allele-specific oligonucleotide-1: 5′ABI_colour-(PRIMER SEQUENCE)-X-3′
2) Allele-specific oligonucleotide-2: 5′ABI_colour-AAAAA-(PRIMER SEQUENCE)-Y-3′
3) Joining-oligonucleotide: 5′-P-PRIMER-3′

The allele discriminating primer were selected from the upstream flanking sequence of the SNP, including the SNP point, and end labeled with a fluorescent dye compatible with the ABI 377 Automated Sequencer machine (tamra, fam or tet). Both allele specific oligonucleotide 1 (AS1) and AS2 were labeled with the same dye. The X and Y at the 3′ end of AS1 and AS2, respectively, indicate the nucleotide discriminating the SNP. The AS2 oligonucleotide has a five adenine nucleotide extension in order to allow discrimination of the OLA products and, thereby, the two genotypes. The joining oligonucleotide is labeled with a phosphate group in its 5′ end in order to make a subsequent ligation possible.

Amplicons containing the SNP were produced using the PCR primers designed at the initial, SNP isolation, step as described in Example 1 above, followed by an Exo-sap purification also as described in Example 1.

The OLA reactions took place in a total volume of 10 μl with the following reagents: 0.25 μl ligase (Pfu DNA ligase, Stratagene, 4 U/μl), 1 μl 10× ligase buffer (Stratagene), 2.5 μl PCR product (purified by exo-sap), 0.5 μl allele-specific oligonucleotide 1 (150 fmol/μl), 0.5 μl allele-specific oligonucleotide 2 (150 fmol/μl) and 0.5 μl joining oligonucleotide (150 fmol/μl) with the following temperature profile: an initial denaturation step of 94_C (10 seconds) then 25 cycles of 95_C (30 seconds) and 55_C (1 min) on a GeneAmp 9600 from ABI. Equal amount of OLA products and formamide gel loading buffer was mixed and loaded onto 6% SequaGel® (National Diagnostics) and ran on ABI 377 Automated Sequencer (ABI) and analysed using GenScan software (ABI).

### EXAMPLE 3

### Isolation of Microsatellite Markers from Atlantic Salmon, Tilapia, Cod, Atlantic Halibut, Seabass

This example describes isolation of genomic DNA containing microsatellite markers from an Atlantic salmon individual, a Nile tilapia individual, a Cod individual, an Atlantic halibut individual, a Seabass individual.

The procedure for isolation of microsatellite containing DNA was identical for each species. The procedure set forth below describes the isolation from one species.

A genomic library was constructed using the following procedure. Genomic DNA was digested with restriction enzyme Sau 3A (Gibco BRL) followed by electrophoresis in a 1% TBE agarose gel. Using 1 Kb DNA size ladder (Amersham Pharmacia), DNA fragments of the size range 900-1100 bp were excised from the gel and isolated using QIAquick Gel extraction kit (Qiagen). The isolated DNA fragments were then ligated to Ready-to-Go pUC18 (Amersham Pharmacia), linearized with BamHI, BAP treated and formulated with T4 DNA ligase, followed by transformation into E. coli Pack Gold supercompetent cells (Stratagene). Cells from the libraries were grown on LA amp agar plates in 37° C. for 12 hours. A colony replica of each plate were done using Colony/Plaque Screen NEF 990A filters (DuPont, Laborel) using the following procedure:

Each filter was uniquely marked with pencil and placed on top of the colony plate. The filters were subsequently stabbed with needle at three locations in order to optimize later orientation of autoradiograms/LA plates.

The filters were lifted from the colony plates and placed on 3 ml 0.5 M NaOH pools for denaturation of colony/DNA for 2 min before placed for 1 min on 3MM filter paper for short drying. The denaturation step was then repeated once before neutralization of filter on 3 ml 1 M Tris (pH 7.5) for 2 min, 1 min of short drying on 3MM filter before one repetition of neutralization step. Filters were air dried in 65° C. for 30 min. for fixation of DNA before washing in 2×SSC, 0.5% SDS, 50° C. Filters were pre hybridized in 120 ml 20×SSC, 24 ml 10% SDS, 24 ml Denhards, 6 ml tRNA 10 mg/ml, 306 ml H₂O for 30 min. in 50° C. before P³² (Amersham) end labeled probe was added and this hybridization step continued in 50° C. for 12 hours. The probe was a (GT)₁₀ oligonucleotide (synthesized at MWG, Germany). Filters were washed twice in 2×SSC, 0.5% SDS, 15 min. room temp. and twice in 0.5×SSC, 0.5% SDS, 50° C., briefly dried at 3MM filter paper, wrapped in plastic film before placing film (Hyperfim ™ MP, Amersham) on top of the filters and placed in −70° C. for about 5 hours. The film was developed using Curix60 developer machine (AGFA) following the supplied recommended protocol and colonies at the original plates containing GT microsatellites were identified. Colonies were picked and transferred to new LA amp agar plates from which over night cultures were produced in LB amp media. Plasmids were isolated using QIAprep Spin Miniprep kit (Qiagen) followed by sequencing of the clone insert using standard M13 forward and reverse sequencing primers, Big Dye Terminator Sequencing kit (ABI) following the supplied recommended protocol and detecting/analyzing the sequence on a 377 Automated Sequencer from ABI.

PCR primers flanking the (GT)ₙ repeat were designed using the Primer3 software (http://www-genome.wi.mit.edu/cgi-bin/primer/primer3_www.cgi)

Primers were ordered from and synthesized at MWG, Germany. One of the primers in each PCR set was labeled at its 5′ end by the primer synthesizing company with dyes that enables subsequent analysis using Automated sequencing machinery (ABI 377).

### EXAMPLE 4

### Determination of Microsatellite Variation in Atlantic Salmon, Tilapia, Cod, Atlantic Halibut, Seabass

This example describes the analysis of microsatellite variation in Atlantic, Nile tilapia, Cod, Atlantic halibut and Seabass by PCR followed by analysis on automated DNA sequencing/analyzing machine (ABI 377).

The procedure was identical for each species. The procedure set forth below describes such variation determination from one species.

Genomic DNA from 20 unrelated fishes was genotyped for a given microsatellite marker in order to detect the level of polymorphism as well as study how efficient (and the quality) each microsatellite marker was amplified by PCR. The PCR reaction took place in a total volume of 20 μl, consisting of 100 ng DNA, 5 μmol of each primer, 2 μl DNTP (2 mM), 2 μl 10×PCR buffer (supplied by ABI optimized for the enzyme), 0.2 μl Ampli-taq polymerase (ABI). Temperature cycling was performed with an initial denaturation step of 95° C. for 3 minutes, then 12 cycles of 95° C. for 30 seconds, 58° C. for 30 seconds and 72° C. for 30 seconds, then 25 cycles at 95° C. for 30 seconds each and 68° C. for 1 minute. Amplification was performed on a GeneAmp 9600 from ABI.

### EXAMPLE 5

### Parentage Testing of Fish

This example describes the usage of microsatellite markers for assignment of individuals to the correct parent pair. The procedure for such typing and assignment is identical for all species. Thus, for exemplification, the procedure given below describes such analysis for Atlantic salmon.

Genomic DNA from 6 female and 4 male breeders were genotyped for a total of 8 microsatellites (SEQ ID NOS:) according to the procedure set forth in Example 4. It was known which male that was crossed to which females. Furthermore, genomic DNA was isolated from a total of 13 offspring and these individuals were subsequently genotyped for the same set of markers as the group of potential parents. The genotyping results are presented in table 1.

TABLE 1
Genotypes of 13 Atlantic salmon offspring, 4 male parents and 6 female partners for 8 microsatellite markers.

| | Marker: | | | | | | | | | | | | | | | |
|---|---|---|---|---|---|---|---|---|---|---|---|---|---|---|---|---|
| | 104 | 104 | 106 | 106 | 109 | 109 | 115 | 115 | 125 | 125 | 131 | 131 | 135 | 135 | 173 | 173 |
| Offspring | | | | | | | | | | | | | | | | |
| B001F06 | 193 | 201 | 244 | 246 | 149 | 157 | 119 | 119 | 152 | 160 | 196 | 200 | 382 | 398 | 229 | 278 |
| B002A03 | 193 | 221 | 246 | 246 | 149 | 153 | 0 | 0 | 152 | 160 | 200 | 200 | 380 | 380 | 239 | 278 |
| B001F10 | 207 | 221 | 246 | 246 | 149 | 151 | 119 | 119 | 160 | 162 | 196 | 196 | 380 | 380 | 229 | 229 |
| B002C03 | 201 | 221 | 246 | 246 | 151 | 153 | 125 | 125 | 160 | 160 | 196 | 196 | 380 | 380 | 229 | 229 |
| B004B01 | 201 | 201 | 246 | 246 | 149 | 151 | 125 | 125 | 152 | 160 | 196 | 196 | 380 | 380 | 229 | 239 |
| B002E08 | 201 | 201 | 246 | 246 | 153 | 153 | 119 | 119 | 152 | 160 | 196 | 204 | 380 | 380 | 229 | 278 |
| B003C07 | 201 | 201 | 246 | 248 | 151 | 153 | 119 | 119 | 152 | 160 | 196 | 204 | 380 | 380 | 229 | 278 |
| B007B07 | 201 | 201 | 246 | 246 | 149 | 153 | 119 | 121 | 152 | 160 | 196 | 204 | 380 | 380 | 229 | 278 |
| B003H09 | 201 | 201 | 246 | 246 | 149 | 151 | 123 | 125 | 152 | 162 | 196 | 204 | 380 | 398 | 237 | 251 |
| B003B08 | 201 | 213 | 246 | 246 | 149 | 157 | 119 | 119 | 152 | 162 | 196 | 196 | 380 | 380 | 278 | 278 |
| B006B05 | 201 | 207 | 246 | 246 | 149 | 157 | 121 | 121 | 152 | 162 | 196 | 204 | 380 | 380 | 237 | 249 |
| B007G05 | 207 | 213 | 246 | 246 | 149 | 149 | 121 | 121 | 152 | 162 | 196 | 196 | 380 | 380 | 237 | 249 |
| B008C02 | 203 | 213 | 246 | 246 | 149 | 153 | 119 | 123 | 152 | 154 | 196 | 204 | 380 | 380 | 229 | 245 |
| Males | | | | | | | | | | | | | | | | |
| P01-E09 | 181 | 193 | 246 | 246 | 149 | 153 | 0 | 0 | 152 | 160 | 200 | 202 | 380 | 382 | 229 | 239 |
| P01-E10 | 201 | 207 | 246 | 246 | 149 | 153 | 119 | 125 | 160 | 162 | 196 | 196 | 380 | 380 | 229 | 239 |
| P01-E11 | 201 | 207 | 246 | 246 | 149 | 149 | 119 | 121 | 160 | 162 | 196 | 196 | 380 | 380 | 237 | 278 |
| P01-E12 | 189 | 203 | 246 | 246 | 149 | 153 | 123 | 125 | 152 | 162 | 200 | 204 | 380 | 380 | 237 | 245 |
| Females | | | | | | | | | | | | | | | | |
| P01-A07 | 201 | 221 | 244 | 246 | 149 | 157 | 119 | 119 | 152 | 162 | 196 | 200 | 380 | 398 | 237 | 278 |
| P02-A03 | 201 | 221 | 246 | 246 | 151 | 153 | 0 | 0 | 152 | 160 | 196 | 204 | 380 | 380 | 229 | 229 |
| P02-D10 | 201 | 221 | 246 | 248 | 151 | 153 | 125 | 125 | 152 | 152 | 204 | 204 | 380 | 380 | 237 | 278 |
| P02-A05 | 173 | 201 | 246 | 246 | 151 | 153 | 121 | 125 | 152 | 152 | 204 | 204 | 380 | 398 | 251 | 278 |
| P01-B06 | 201 | 213 | 246 | 246 | 153 | 155 | 121 | 125 | 154 | 160 | 196 | 196 | 380 | 380 | 229 | 278 |
| P02-E08 | 201 | 213 | 246 | 246 | 149 | 157 | 0 | 0 | 152 | 152 | 196 | 204 | 380 | 380 | 249 | 278 |

A comparison analysis was performed between the genotypes of the offspring and the potential parent pairs and the correct parent pair was identified based on their ability to produce an offspring with the same genotype as found in a particular offspring. The result of such parent pair assignment of the offspring genotyped is presented in table 2.

TABLE 2
Assignment of offspring to parent pair

| ID | SIRE | DAM |
|---|---|---|
| B001F06 | P01-E09 | P01-A07 |
| B002A03 | P01-E09 | P01-A07 |
| B001F10 | P01-E10 | P02-A03 |
| B002C03 | P01-E10 | P02-A03 |
| B004B01 | P01-E10 | P02-A03 |
| B002E08 | P01-E10 | P02-D10 |
| B003C07 | P01-E10 | P02-D10 |
| B007B07 | P01-E10 | P02-D10 |
| B001C12 | P01-E11 | P02-A05 |
| B003H09 | P01-E11 | P02-A05 |
| B004D04 | P01-E11 | P02-A05 |
| B003B08 | P01-E11 | P02-E08 |

This example demonstrates an assignment analysis of a small number of offspring/families. The same procedure is used for identify the correct parent pair in a situation where any number of offspring/samples are to be assigned to correct parent pair identified from any size of potential male and female parent individual group available.

This example is shown for microsatellite markers. Identical tests can be performed by using other genetic markers as for example SNPs.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

We claim:

1. An isolated nucleic acid molecule, comprising a single nucleotide polymorphism (SNP) selected from the group consisting of:
(a) a nucleic acid molecule having a nucleotide sequence selected from the group set forth in FIG. 1; and
(b) a nucleic acid molecule having a nucleotide sequence that hybridizes to the nucleotide sequence of (a) or its complement under highly stringent hybridization conditions.

2. An isolated oligonucleotide comprising at least 17 contiguous nucleotides of a nucleotide sequence set forth in FIG. 1, or the complement thereof.

3. The isolated oligonucleotide of claim 2, labeled with a detectable marker.

4. A primer pair suitable for use in the polymerase chain reaction (PCR), comprising two oligonucleotides according to claim 2 and capable of amplifying a nucleotide sequence selected from the group set forth in FIG. 1.

5. The primer pair of claim 4, wherein said oligonucleotides are selected from the group set forth in FIGS. 1 and 2.

6. An isolated nucleic acid molecule, comprising a single nucleotide polymorphism (SNP) selected from the group consisting of:
(a) a nucleic acid molecule having a nucleotide sequence selected from the group set forth in FIG. 4; and
(b) a nucleic acid molecule having a nucleotide sequence that hybridizes to the nucleotide sequence of (a) or its complement under highly stringent hybridization conditions.

7. An isolated oligonucleotide comprising at least 17 contiguous nucleotides of the nucleotide sequence set forth in FIG. 4, or the complement thereof.

8. The isolated oligonucleotide of claim 7, labeled with a detectable marker.

9. A primer pair suitable for use in the polymerase chain reaction (PCR), comprising two oligonucleotides according to claim 7 and capable of amplifying a nucleotide sequence selected from the group set forth in FIG. 4.

10. The primer pair of claim 9, wherein said oligonucleotides are selected from the group set forth in FIG. 4.

11. A method for detecting a nucleic acid molecule comprising a single nucleotide polymorphism in a sample, comprising contacting said sample containing nucleic acids with one or more oligonucleotides according to claims 2 or 7, wherein said contacting is effected under high stringency hybridization conditions, and identifying a nucleic acid that hybridizes to said oligonucleotide.

12. A method for detecting a nucleic acid molecule comprising a single nucleotide polymorphism in a sample, comprising contacting said sample with the primer pair of claim 4 or 9, amplifying a nucleic acid molecule using polymerase chain reaction, and detecting said amplification.

13. An isolated nucleic acid molecule, comprising a microsatellite sequence selected from the group consisting of:
(a) a nucleic acid molecule having a nucleotide sequence selected from the group designated SEQ ID NOS: 309-367; and
(b) a nucleic acid molecule having a nucleotide sequence that hybridizes to the nucleotide sequence of (a) or its complement under highly stringent hybridization conditions.

14. An isolated oligonucleotide comprising at least 17 contiguous nucleotides of a nucleotide sequence selected from the group designated SEQ ID NOS: 309-367, or the complement thereof.

15. The isolated oligonucleotide of claim 14, labeled with a detectable marker.

16. A primer pair suitable for use in the polymerase chain reaction (PCR), comprising two oligonucleotides according to claim 14.

17. An isolated nucleic acid molecule, comprising a polymorphic sequence selected from the group consisting of:
(a) a nucleic acid molecule having a nucleotide sequence selected from the group designated SEQ ID NOS: 368-373; and
(b) a nucleic acid molecule having a nucleotide sequence that hybridizes to the nucleotide sequence of (a) or its complement under highly stringent hybridization conditions.

18. An isolated nucleic acid molecule, comprising a microsatellite sequence selected from the group consisting of:
(a) a nucleic acid molecule having a nucleotide sequence selected from the group set forth in FIG. 11; and
(b) a nucleic acid molecule having a nucleotide sequence that hybridizes to the nucleotide sequence of (a) or its complement under highly stringent hybridization conditions.

19. An isolated oligonucleotide comprising at least 17 contiguous nucleotides of a nucleotide sequence selected from the group set forth in FIG. 4, or the complement thereof.

20. The isolated oligonucleotide of claim 19, labeled with a detectable marker.

21. A primer pair suitable for use in the polymerase chain reaction (PCR), comprising two oligonucleotides according to claim 19.

22. The primer pair of claim 21, wherein said oligonucleotides are selected from the primer sequences set forth in FIG. 11.

23. An isolated nucleic acid molecule, comprising a single nucleotide polymorphism (SNP) selected from the group consisting of:
(a) a nucleic acid molecule having a nucleotide sequence selected from the group designated SEQ ID NOS: 374-409; and
(b) a nucleic acid molecule having a nucleotide sequence that hybridizes to the nucleotide sequence of (a) or its complement under highly stringent hybridization conditions.

24. An isolated oligonucleotide comprising at least 17 contiguous nucleotides of a nucleotide sequence selected from the group designated SEQ ID NOS: 374-409, or the complement thereof.

25. The isolated oligonucleotide of claim 24, labeled with a detectable marker.

26. A primer pair suitable for use in the polymerase chain reaction (PCR), comprising two oligonucleotides according to claim 24.

27. An isolated nucleic acid molecule, comprising a microsatellite sequence selected from the group consisting of:
(a) a nucleic acid molecule having a nucleotide sequence selected from the group designated SEQ ID NOS: 155-164; and
(b) a nucleic acid molecule having a nucleotide sequence that hybridizes to the nucleotide sequence of (a) or its complement under highly stringent hybridization conditions.

28. An isolated nucleic acid molecule, comprising a polymorphic sequence selected from the group consisting of:
(a) a nucleic acid molecule having a nucleotide sequence selected from the group designated SEQ ID NOS: 410-414; and
(b) a nucleic acid molecule having a nucleotide sequence that hybridizes to the nucleotide sequence of (a) or its complement under highly stringent hybridization conditions.

29. An isolated oligonucleotide comprising at least 17 contiguous nucleotides of the nucleotide sequence selected from the group designated SEQ ID NOS:410-414, or the complement thereof.

30. The isolated oligonucleotide of claim 29, labeled with a detectable marker.

31. A primer pair suitable for use in the polymerase chain reaction (PCR), comprising two oligonucleotides according to claim 29.

32. An isolated nucleic acid molecule, comprising a polymorphic sequence selected from the group consisting of:
(a) a nucleic acid molecule having a nucleotide sequence selected from the group designated SEQ ID NOS: 415-472; and
(b) a nucleic acid molecule having a nucleotide sequence that hybridizes to the nucleotide sequence of (a) or its complement under highly stringent hybridization conditions.

33. An isolated oligonucleotide comprising at least 17 contiguous nucleotides of the nucleotide sequence selected from the group designated SEQ ID NOS: 415-472, or the complement thereof.

34. The isolated oligonucleotide of claim 33, labeled with a detectable marker.

35. A primer pair suitable for use in the polymerase chain reaction (PCR), comprising two oligonucleotides according to claim 34.

36. A method for detecting a nucleic acid molecule comprising a polymorphic sequence in a sample, comprising contacting said sample containing nucleic acids with one or more oligonucleotides according to claims 14, 19, 24, 29, or 33, wherein said contacting is effected under high stringency hybridization conditions, and identifying a nucleic acid that hybridizes to said oligonucleotide.

37. A method for detecting a nucleic acid molecule comprising a microsatellite sequence in a sample, comprising contacting said sample with the primer pair of claims 16, 21, 26, 31, or 35, amplifying a nucleic acid molecule using polymerase chain reaction, and detecting said amplification.

38. A method of determining the population of origin of a fish sample comprising the steps of:
(a) providing an origin genotype database comprising a collection of candidate parent genotypes, wherein each of said candidate parent genotypes represents a distinct population of origin; and
(b) comparing a sample genotype to said candidate parent genotypes, wherein a match between said sample genotype and one of said candidate parent genotypes identifies the population of origin of said sample.

39. A method of determining the origin of a fish sample comprising the steps of:
(a) providing an origin genotype database comprising a collection of candidate genotype profiles, wherein each of said candidate genotype profiles represents a distinct population of origin; and
(b) comparing a sample genotype to said candidate genotype profiles, wherein a match between said sample genotype and one of said candidate genotype profiles identifies the population of origin of said sample.

40. A method of determining the origin of a fish sample comprising the steps of:
(a) providing a parentage genotype database comprising a collection of candidate parent genotypes, wherein each of said candidate parent genotypes represents a distinct origin; and
(b) comparing a sample genotype to said parentage genotype database, wherein a match between said sample genotype and one of said candidate parent genotypes identifies the origin of said sample.

41. The method of claim 40, wherein said parentage genotype database comprises every potential origin genotype.

42. The method of claim 40, wherein said candidate parent genotypes comprise two or more distinct species.

43. The method of claim 40, wherein said sample and candidate parent genotypes belong to the family Salmonidae.

44. The method of claim 40, wherein said sample and candidate parent genotypes belong to the species Salmo salar.

45. The method of claim 40, wherein said sample and candidate parent genotypes belong to the genus tilapia.

46. The method of claim 45, wherein said sample and candidate parent genotypes belong to the species Oreochromis niloticus.

47. The method of claim 40, further comprising sample and candidate parent genotypes belonging to a species selected from the group consisting of rainbow trout, halibut, seabass and Atlantic cod.

48. The method of claim 40, further comprising the initial steps of:
(a) extracting nucleic acid corresponding to each of said distinct populations of origin; and
(b) genotyping the extracted nucleic acid with selected genetic markers to obtain said collection of candidate parent genotypes.

49. The method of claim 48, wherein said nucleic acid is extracted from broodstock individuals.

50. The method of claim 48, wherein said genetic markers are selected from the group consisting of single nucleotide polymorphisms (SNPs), microsatellites, restriction length polymorphisms (RFLPs), amplified fragment length polymorphisms (AFLP), random amplified polymorphic DNA (RAPD), mitochondrial DNA.

51. The method of claim 50, wherein said genetic markers comprise a polymorphic nucleotide sequence selected from the group set forth in FIGS. 1-9 and 11.

52. The method of claim 50, wherein said genetic markers comprise SNPs.

53. The method of claim 52, wherein said SNPs comprise the nucleotide sequences set forth in FIG. 1.

54. The method of claim 52, wherein said SNPs comprise SEQ ID NOS: 165-308.

55. The method of claim 52, further comprising identifying said SNPs by performing an oligonucleotide ligation assay (OLA).

56. The method of claim 52, further comprising identifying said SNPs by performing a hybridization assay.

57. The method of claim 56, wherein said hybridization assay is performed on a DNA chip.

58. The method of claim 40, wherein the absence of said match excludes said candidate genotypes as the origin of said sample.

59. The method of claim 40, further comprising generating a central database capable of storing said population of candidate parent genotypes.

60. The method of claim 40, wherein said central database is capable of instantaneously comparing said sample genotype to said collection of candidate parent genotypes.

61. The method of claim 60, wherein said central database of candidate parent genotypes is on the accessible through the internet.
